(19) United States
(12) Patent Application Publication
(10) Pub. No.: US 2023/0022575 A1
(43) Pub. Date: Jan. 26, 2023

(54) BACTERIAL CONJUGATIVE SYSTEM AND THERAPEUTIC USES THEREOF

(71) Applicant: SOCIÉTÉ DE COMMERCIALISATION DES PRODUITS DE LA RECHERCHE APPLIQUÉE SOCPRA SCIENCES SANTÉ ET HUMAINE, Sherbrooke (CA)

(72) Inventors: Sébastien RODRIGUE, Sherbrooke (CA); Kevin NEIL, Sherbrooke (CA); Nancy ALLARD, Sherbrooke (CA); Vincent BURRUS, Sherbrooke (CA)

(21) Appl. No.: 17/258,893

(22) PCT Filed: Jul. 9, 2019
(86) PCT No.: PCT/CA2019/050945

§ 371 Date: Jan. 8, 2021

Related U.S. Application Data
(60) Provisional application No. 62/696,367, filed on Jul. 11, 2018.

(51) Int. Cl. C12N 15/90; C12N 15/10; C12N 15/70; A61P 31/04

(52) U.S. Cl. CPC C12N 15/907; C12N 15/102; C12N 15/70; A61P 31/04; A61K 35/741

(57) ABSTRACT

The present disclosure concerns the use of a mating pair stabilization module comprising a type IV adhesion pilus with a conjugative bacterial host cell or as a part of a conjugative delivery system for mediating effective in vivo conjugation.

### TECHNOLOGICAL FIELD

The present disclosure relates to a bacterial conjugative system for transferring, in vivo, a nucleic acid cargo from a conjugative bacterium to a recipient bacterium.

### BACKGROUND

Bacterial communities play an important role in human and animal health. It is now clearly established that imbalances in gut microbial populations, also known as dysbiosis, are linked to several severe pathologies such as: cancer, diabetes, Crohn's disease, and irritable bowel syndrome to name only a few. Being able to precisely manipulate bacterial communities to restore and/or maintain healthy microbiomes would therefore be of great interest to cure those diseases. For example, having a technology allowing the selective elimination or inactivation of pathogenic bacteria, while maintaining the beneficial flora of a microbiome intact, would be a highly valuable therapeutic tool. In addition, having the possibility to modify certain bacteria of a microbial community so they can locally provide therapeutic agents to the organ they colonize would also be an important approach for improving human and animal health. In summary, developing a tool that would allow precise in situ manipulation of human or animal microbiomes could unleash new promising therapeutic possibilities.

Parallel to this, public health is also challenged by the alarming emergence of antibiotic resistant bacteria that infects the gut (e.g. Campylobacter, Escherichia coli and Salmonella), the urinary tract (e.g. Escherichia coli), and wounds (e.g. Staphylococcus aureus). This is a major concern because the development of new antibiotic molecules has declined drastically over the last decades. It is estimated that by 2050 antibiotic resistant bacteria will be responsible for more human deaths than cancer. Therefore, to palliate the growing inefficiency of conventional antibiotics, there is an urgent need to develop new alternative drugs to fight antibiotic resistant bacteria.

Bacterial conjugation is a natural process through which a donor bacterium transfers genetic material, via a conjugative element, into a recipient bacterium. Owing to the recent advances in synthetic biology, bacteria (such as probiotics) could be engineered to use bacterial conjugation in order to transfer a genetic cargo containing the CRISPR-cas9 RNA-guided nuclease system into a target bacterium. This new class of drug, based on a probiotic capable of delivering CRISPR-cas9 to target bacteria, could provide an efficient way to manipulate microbiomes, or treat bacterial infections, in situ. For instance, such probiotics could be used to transfer the CRISPR-cas9 RNA-guided nuclease system into target bacteria to delete antibiotic resistance genes or to eliminate pathogenic bacteria by inducing double-strand breaks in their chromosomes. This principle could also be directly applied to the treatment of dysbiosis by targeting over-represented species of bacteria, hereby editing the microbiome with great precision.

While engineering bacteria, including probiotics, that use bacterial conjugation to deliver CRISPR-cas9 system to modify microbiomes is a promising avenue, this approach faces some serious technological challenges that need to be addressed. As a matter of facts, to be useful, this technology requires that: (1) the engineered probiotic is capable of carrying out bacterial conjugation in vivo inside the environment of human or animal body (e.g. in the gut, the urinary tract, or a wound), and (2) the in vivo conjugation efficiency must be high enough to achieve satisfactory therapeutic effects.

Up to now, bacterial conjugation has been studied almost exclusively in vitro in Petri dishes, an environment that significantly differs from the conditions encountered in vivo in a human or animal body. Very little is known about which conjugative bacterial systems are actually capable of functioning in vivo, and at what efficiency. In stark contrast to drugs derived from chemical molecules (e.g. traditional antibiotics), for which the in vitro activity in a Petri dish is indicative of the in vivo activity, drugs based on living organisms (e.g. bacteria) are not as predictable. For instance, contrary to inert chemical molecules, bacteria are living organisms that are adapted to certain conditions, and that respond to their environment via complex mechanisms affecting a plethora of cellular processes. Therefore, it is difficult to predict if a bacterium capable of conjugation in vitro will be able to perform conjugation under in vivo conditions. In short, for drugs that use living organisms as therapeutic vectors, the in vitro efficacy is not sufficient to predict the ability of the drug to function in vivo.

In sum, a new class of therapeutics based on bacterial conjugation is a very promising therapeutic avenue to manipulate bacterial communities in situ. However, in order to become a viable approach, this technology requires the development of a bacterial system actually capable of carrying out conjugation in vivo, and this, with high-efficiency. Such bacterial system can then be used as a universal platform for the transfer and delivery of CRISPR-cas9, or any other type of genetic cargo that can eliminate or modify target bacteria.

### BRIEF SUMMARY

According to a first aspect, the present disclosure provides a conjugative bacterial host cell for transferring, in vivo, a genetic cargo to a recipient bacterial cell. The conjugative bacterial host cell comprises (i) the genetic cargo (wherein the genetic cargo comprises a transport module operatively associated with a payload module); (ii) a type IV secretion system module, (iii) a mating pair stabilization module comprising a type IV adhesion pilus, the type IV adhesion pilus comprising an adhesin; and (iv) a mobilization module. The transport module is capable of being recognized by the transport machinery encoded by the mobilization module. In an embodiment, the type IV adhesion pilus and/or the adhesin comprises at least one of the following proteins: PilL, PilN, PilO, PilP, PilQ, PilR, PilS, PilT, TraB, PilU, PilV or TraN. In another embodiment, the type IV adhesion pilus is derived from at least one of the following family of bacterial plasmids: IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 or Inc18. In still another embodiment, the type IV secretion system module comprises at least one of the following proteins: VirB1, VirB2, VirB3, VirB4, VirB5, VirB6, VirB7, VirB8, VirB9, VirB10, VirB11 or VirD4. In yet another embodiment, the type IV secretion system module is derived from at least one of the following family of bacterial plasmids: MPF_{T}, MPF_{F}, MPF_{I}, MPF_{FATA}, MPF_{B}, MPF_{FA}, MPF_{G} or MPF_{C}. In yet a further embodiment, the genetic cargo is located on a first extrachromosomal vector and further comprises a first vegetative replication module, the conjugative bacterial host cell comprises a first maintenance module encoding a first replication machinery, and the first vegetative replication module is capable of being recognized by the first replication machinery encoded by the first maintenance module. In such embodiment, the first maintenance module comprises at least one of the following proteins: RepA, ParA, ParB, a DNA primase, YgiA, a toxin, Vcrx028, YcfA, an antitoxin, Vcrx027, YcfB, a DNA topoisomerase, YdiA or YdgA. In still another embodiment, the first vegetative replication module or the first maintenance module is derived from at least one of the following family of bacterial plasmids: IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 or Inc18. In still a further embodiment, the genetic cargo comprises the mobilization module. In an embodiment, the conjugative bacterial host cell comprises a transfer machinery located on a second extrachromosomal vector, wherein the transfer machinery comprises the type IV secretion system module, the mating pair stabilization module and a second vegetative replication module; the conjugative bacterial host cell comprises a second maintenance module encoding a second replication machinery; and the second vegetative replication module is capable of being recognized by the second replication machinery machinery encoded by the second maintenance module. In an embodiment, the second maintenance module comprises at least one of the following proteins: RepA, ParA, ParB, a DNA primase, YgiA, a toxin, Vcrx028, YcfA, an antitoxin, Vcrx027, YcfB, a DNA topoisomerase, YdiA or YdgA. In a further embodiment, the second vegetative replication module or the second maintenance module is derived from at least one of the following family of bacterial plasmids: IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 or Inc18. In an embodiment, the transfer machinery further comprises the mobilization module. In yet another embodiment, the conjugative bacterial host cell comprises a transfer machinery located in the bacterial chromosome, wherein the transfer machinery comprises the type IV secretion system module, the mating pair stabilization module and the mobilization module. In an embodiment, the conjugative bacterial host cell further comprises an exclusion module, a selection module and/or a regulatory module. In an embodiment, the regulatory module comprises at least one of the following regulatory protein or non-coding RNA: YajA, YafA, FinO, Fur, Fnr, KorA, AcaC, AcaD, Acr1, Acr2, StbA, TwrA, ResP, KfrA, ArdK, dCas9, crRNA, ZFN, TALEN, taRNA, toehold switch, AraC, TetR, LacI or Laclq. In another embodiment, the mobilization module comprises at least one of the following proteins: VirC1, NikB or NikA. In still another embodiment, the mobilization module is derived from at least one of the following family of bacterial plasmids: MOB_{F}, MOB_{P}, MOB_{V}, MOB_{H}, MOB_{C} or MOB_{Q}. In a further embodiment, the mating pair stabilization module further comprises a shufflase for modifying a shufflon associated with the gene encoding the adhesin. In still another embodiment, the shufflon is derived from at least one of the following family of bacterial plasmids: IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 and/or Inc18. In a further embodiment, the shufflase is encoded by a rci gene. In a further embodiment, the sufflase is derived from at least one of the following family of bacterial plasmids: IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 and/or Inc18. In still a further embodiment, the payload module encodes a nuclease. In yet another embodiment, the nuclease is a clustered regularly interspaced short palindromic repeat (CRISPR) associated DNA-binding (Cas) protein or a Cas protein analog and the payload module further encodes a CRISPR RNA (crRNA) molecule recognizable by the Cas protein or the Cas protein analog. In still another embodiment, the crRNA molecule is substantively complementary to a DNA molecule in the recipient bacterium. In yet a further embodiment, the DNA molecule corresponds to a gene in the recipient bacterium. In an embodiment, the gene encodes a virulence factor in the recipient bacterium. In still another embodiment, the payload module further encodes a trans-activating CRISPR RNA recognizable by the Cas protein or the Cas protein analog. In another embodiment, the payload module encodes a therapeutic protein. In another embodiment, the therapeutic protein allows for the production or the degradation of a metabolite. In an embodiment, the conjugative bacterial host cell has an in vivo conjugation efficiency of at least 10⁻³ bacterial transconjugant/recipient CFU and/or a ratio of in vitro conjugation efficiency obtained in a liquid medium when compared to a corresponding conjugation efficiency obtained in a solid medium higher than 0.1%. In still another embodiment, the conjugative bacterium is a probiotic bacterium. In a further embodiment, the conjugative bacterium is an enteric bacterium. In some embodiment, the conjugative bacterial host cell is from the genus Escherichia, for example, from the species Escherichia coli and in some specific embodiments, from the strain Escherichia coli Nissle 1917.

According to a second aspect, the present disclosure provides a composition comprising the conjugative bacterial host defined herein and an excipient. In some embodiments, the composition is formulated for oral administration.

According to a third aspect, the present disclosure provides a process for making the conjugative bacterial host cell defined herein, the process comprises introducing the genetic cargo and at least one of the type IV secretion system module, the mating pair stabilization module or the mobilization module defined herein in a bacterium to provide the conjugative bacterial host cell. In some embodiments, the process further comprises introducing at least one of the vegetative replication module, the maintenance module, the regulatory module, the selection module or the exclusion module as defined herein in the bacterium to provide the conjugative bacterial host cell.

According to a fourth aspect, the present disclosure provides a conjugative recombinant bacterial host cell obtainable or obtained by the process described herein.

According to fifth aspect, the present disclosure provides a process for making the composition defined herein, the process comprising formulating the conjugative bacterial host cell defined in herein with an excipient.

According to a sixth aspect, the present disclosure provides a composition obtainable or obtained by the process described herein.

According to a seventh aspect, the present disclosure provides a conjugative recombinant bacterial host cell defined herein or a composition defined herein for transferring, in vivo in a subject suspected of having a recipient bacterium, the genetic cargo from the conjugative bacterial host cell to the recipient bacterium. The present disclosure also provides the use of a conjugative recombinant bacterial host cell defined herein or a composition defined herein for transferring, in vivo in a subject suspected of having a recipient bacterium, a genetic cargo from the conjugative bacterial host cell to the recipient bacterium. The present disclosure further provides the use of a conjugative recombinant bacterial host cell defined herein or a composition defined herein for the manufacture of a medicament for transferring, in vivo in a subject suspected of having a recipient bacterium, a genetic cargo from the conjugative bacterial host cell to the recipient bacterium. The present disclosure also provides a method for transferring, in vivo in a subject suspected of having a recipient bacterium, a genetic cargo from a conjugative bacterial host cell to the recipient bacterium, the method comprising administering an effective amount of a conjugative recombinant bacterial host cell defined herein or a composition defined herein to the subject under conditions to allow the transfer of the genetic cargo to the recipient bacterium. In some embodiments, the conjugative bacterial host cell is a probiotic bacterial host cell and/or an enteric bacterium. In another embodiment, the modification system of the conjugative bacterial host cell is substantially similar to the restriction system of the recipient bacterium. In another embodiment, the payload module encodes a heterologous protein, such as for example a therapeutic protein, a heterologous protein allowing for the production or the degradation of a metabolite, and/or a nuclease. In an embodiment, the nuclease is a clustered regularly interspaced short palindromic repeat (CRISPR) associated DNA-binding (Cas) protein and the payload module further encodes a guide RNA (gRNA) molecule recognizable by the Cas protein. In some embodiments, the gRNA molecule is substantively complementary to a DNA molecule in the recipient bacterium. In a further embodiment, the DNA molecule is a gene in the recipient bacterium. In yet a further embodiment, the gene encodes, in the recipient bacterium, a virulence factor, a protein involved in a resistance to an antibiotic, a toxin or a pilus. In some embodiments, the conjugative bacterial host cell can be used for the treatment or the alleviation of symptoms of a dysbiosis or an infection caused by the recipient bacterium.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1.A to D. Generation of Escherichia coli Nissle 1917 (EcN) strain derivatives required to easily distinguish the donor and recipient strains in conjugation experiments. The donor strain, termed KN11, was generated by insertion of the first cassette, including a specialized metagenomics sequencing (16S) tag and aad7, a spectinomycin resistance gene (FIG. 1.A). KN02 harbored a different cassette containing a different metagenomics sequencing (16S) tag, strAB for streptomycin resistance, an IPTG inducible NeonGreen fluorescent reporter, and the cat gene conferring resistance to chloramphenicol (FIG. 1.B). KN02 was used both as a recipient and as a target strain. KN03 had an insert that contains strAB for streptomycin resistance, the same metagenomics sequencing (16S) tag as KN02 and tetB for tetracycline resistance (FIG. 1.C). KN03 was used as both a recipient and a non-target control. All genes in FIG. 1.A to C are shown to scale with the total amount of base pair (bp) shown below each DNA construct. All inserts were cloned into pGRG36's SmaI and XhoI restriction sites located between attL_{Tn7} and attR_{Tn7} sites. (FIG. 1.D) The resulting plasmids were transformed in EcN and the expression of the Tn7 system was induced with arabinose. This led to the excision of the DNA fragment located between the attL/R_{Tn7} of pGRG36, which was was next integrated at the 3′ end of the glmS gene. Since the pGRG36 replication machinery is heat-sensitive, an incubation at a non-permissive temperature of 42° C. was finally used to cure the empty vector backbone from the cell.

FIG. 2. Impact of dapA deletion on E. coli's metabolism. The deletion of dapA prevents the transformation of L-asparate-semialdehyde into 4-hydroxy-2,3,4,5-tetrahydrodipicolinate (THDP-OH). This reaction is essential for synthesis of both forms of DAP in E. coli. L,L-DAP and meso-DAP can however be imported from the environment to complement the mutation and allow the synthesis of both peptidoglycan and lysine. This makes DAP an essential medium additive for growth of the dapA deletion mutants. Abbreviations and designations: THDP-OH: 4-hydroxy-2,3,4,5-tetrahydrodipicolinate; THDP, (S)-2,3,4,5-tetrahydrodipicolinate; Succinyl-AKP, N-succinyl-L-2-amino-6-ketopimelate; Succinyl-DAP, N-succinyl-L,L-2,6-diaminopimelate; L,L-DAP, LL-2,6-diaminopimelate; meso-DAP, meso-2,6-diaminopimelate; DapA, dihydrodipicolinate synthase; DapB, dihydrodipicolinate reductase; DapD, THDPA succinylase; SerC, succinyl-DAP aminotransferase; DapE, succinyl-DAP desuccinylase; DapF, DAP epimerase; LysA, DAP decarboxylase.

FIGS. 3.A to G. Streptomycin (Sm) treatment improves mouse gut colonization by Escherichia coli Nissle 1917 (EcN). The ability of different Sm concentrations to deplete endogenous Enterobacteriaceae (white area) and to promote EcN colonization (gray area) was evaluated by quantifying CFU in feces from mice treated with 0 mg/L (FIG. 3.A), 50 mg/L (FIG. 3.B), 100 mg/L (FIG. 3.C), 250 mg/L (FIG. 3.D), 450 mg/L (FIG. 3.E) and 1000 mg/L (FIG. 3.F) in drinking water starting at day −2. The colonization levels of KN01 in several sections of the intestinal tract of untreated (0) and streptomycin-treated ( ) mice was evaluated (FIG. 3.G). The dotted line in panel G indicates sufficient CFU levels for conjugation frequency evaluation. This was based on the minimal in vitro conjugation rate on solid support for all tested systems (about 1×10⁻³ transconjugants/recipient). Horizontal black lines show the mean values under each condition.

FIGS. 4.A to E. Evaluation of transfer efficiencies for conjugative plasmid candidates. Six different conjugative plasmids were first tested for transfer efficiency both on agar (solid) and in broth (liquid) for 2 hours at 37° C. (FIG. 4.A). Conjugative plasmids were also tested for their transfer efficiency in the murine gut using 4 mice per experiment. The proportion of transconjugants per recipient bacteria was evaluated in feces (FIG. 4.B) throughout 3 days and compared to the ratio found in the caecum at day 3 (FIG. 4.C). TP114's ability to transfer was confirmed using an additional set of 4 mice, and compared to the conjugation frequencies obtained in vitro on agar for the same time points (FIG. 4.D). Conjugation experiments with TP114 and R6K in both Sm treated and untreated mice were performed to compare transfer efficiencies within partly depleted or intact microbiota. Transfer rates were measured in the feces after 12 hours of conjugation in 4 mice per conditions per plasmid (FIG. 4.E). Error bars show standard deviation of the mean (black lines) from at least 3 biological replicates.

FIGS. 5.A to F. Raw colony forming units (CFUs) data used to calculate in vivo transfer rates showed in FIG. 4.B. The CFUs for donors (•), recipients (▪) and transconjugants (▾) bacteria were counted from feces samples at day 1, 2, 3 on selective MacConkey agar plates for each plasmid. CFUs from conjugation of pOX38 (FIG. 5.A), R6K (FIG. 5.B), TP114 (FIG. 5.C), pVCR94 (FIG. 5.D), R388 (FIG. 5.E) and RK24 (FIG. 5.F) are shown.

FIGS. 6.A to D. Effect of time between recipient and donor strains colonization on conjugation efficiencies. Sm-treated mice were fed with the recipient bacteria either 2 or 12 hours before the introduction of the donor strain. Conjugation efficiency of TP114 (FIG. 6.A) and R6K (FIG. 6.B) was followed throughout four days in the feces of 4 mice to evaluate the impact of recipient bacteria colonization prior to conjugation. The recipient's colonization was also followed with CFU from feces throughout the experiment with both TP114 (FIG. 6.C) and R6K (FIG. 6.D). The time points are given relative to the donor strain introduction at time=0 day.

FIG. 7. Gene annotation of TP114 as predicted by RAST, CDsearch and BLAST. TP114 was sequenced using both Illumina and Oxford Nanopore technologies and coding genes were first annotated using RAST. A locus tag was attributed to each CDS with the prefix TP114-0 and a number referring to gene order based on the starting position of TP114's sequence as deposited on genbank: MF521836.1. The annotation was further refined using CDsearch and BLAST to attribute putative functions to the genes. Names were attributed to genes based on their putative homolog. General functions were then manually attributed to different modules that mediate a specific function such as type 4 secretion system (T4SS), mating pair stabilization, mobilization, maintenance, regulation, selection and unknown function.

FIGS. 8.A to D. Sequence homology between TP114 and other plasmids of the IncI family. Sequence homology was evaluated using BRIGG, a BLAST-like program that shows sequence identity in circular pattern. Sequence identity threshold were set at 100%, 70% and 50% for all analysis. TP114's sequence was first compared to seven members of the IncI2 subfamily based on the nucleic acid sequence (FIG. 8.A), and the amino acid sequence of its coding genes (FIG. 8.B). Gene conservation among IncI2 plasmids is also compiled in Table 6. TP114 was then compared to seven members of the IncI1 subfamily based on the nucleic acid sequence (FIG. 8.C), and the amino acid sequence of its coding genes (FIG. 8.D). Homology regions for the IncI1 subfamily only comprised the repA replication initiation gene, the shufflon and its associated shufflase rd. Numbers on homology rings correspond to the plasmids in the legend with 1 being the innermost ring and 7 being the outermost ring.

FIG. 9 High-density transposon mutagenesis (HDTM) experiment overview. An EcN containing TP114 was bombarded with transposons using MFDpir+ containing pFG051 (SEQ ID NO: 147) (a mobilizable Tn5 transposition plasmid) and pFG036 (SEQ ID NO: 146) (a plasmid repressing Tn5 transposon machinery in the donor strain). This resulted in the random insertion of Tn5 in both EcN's chromosome and TP114, generating HDTM Library 1. A conjugation experiment both in vitro (on solid support) and in vivo (in the murine gut) was performed to select for TP114::Tn5 mutants that were still able to transfer in those specific environments. The in vivo transfer of HDTM Library 1 was carried for 2 days in two mice per biological replicate Two HDTM libraries were generated from this experiment: transconjugants retrieved from feces samples constituted the HDTM Library 4, whereas HDTM Library 6 is composed of transconjugants retrieved from the caecum of the mice. The in vitro transfer resulted in HDTM Library 2 which was again used as donors for in vitro solid mating (generating the HDTM Library 3) and in vivo conjugation in the gut (generating the HDTM Library 5 and 7 consisting of transconjugants found in the feces and in the caecum respectively). HDTM Library 8 was generated by conjugation of TP114::tetB (SEQ ID NO: 166) in HDTM Library 2 to investigate the exclusion mechanism of TP114.

FIG. 10 HDTM library analysis. HDTM libraries were sequenced and reads were used to precisely locate transposon insertion sites in TP114. Read mapping was then visualized the using UCSC Genome Browser. Black lines represents an insertion site, the height of the line represents the density of reads at a given position in TP114. Tracks shown are representative of the background noise found in the HDTM library 2 as compared with library 3. Data of biological replicate #2 is shown for HDTM Libraries 1, 2 and 3.

FIG. 11. Correlation between HDTM samples. The normalized number of reads mapped onto each 100 bp bin on TP114 was correlated between replicates and conditions using Pearson correlation. A grayscale was applied to the data in order to visually identify the samples which strongly correlates (1.0 in dark grey) or weakly correlates (0.0 in white) between each other. Samples were identified following a three numbers format X.X.X, where the first position refers to the HDTM library number, the second position refers to the biological replicate of the HDTM library, and the third number refers to the mouse identity when experiments were in vivo.

FIG. 12. Essential genes for plasmid maintenance. HDTM libraries were sequenced and reads were mapped based on their insertion point on TP114. Read mapping was then visualized using the UCSC Genome Browser. Vertical lines represent transposon insertion sites, with their respective height corresponding the density of reads at this position. The selected racks presents three biological replicates of HDTM library 1 that were analyzed for any reproducible drop in transposon insertion coverage. These low coverage regions were considered to represent essential maintenance genes. However, some genes contained low mappability regions, which also appeared as low coverage regions and were filtered out of the analysis. The remaining genes with low coverage are are shown within a dotted frame (see also Table 8) and considered important for plasmid maintenance.

FIGS. 13.A to D. Distribution of gene count ratios of HDTM libraries 2 and 3. This procedure was repeated to determine gene importance in HDTM library 4 to 7. Gene counts were first determined by calculating the normalized number of reads mapping within a given gene under a specific condition. Gene counts were then compared to the gene counts in HDTM Library 1 using the formula (gene count in condition X—gene count in condition 1)/gene count in condition 1. Max and average values, black and gray lines respectively, were calculated using a set of genes suspected to be essential in the test Library X but not in Library 1. The gene count ratio distribution is shown for HDTM Library 2 (FIG. 13.A) with the dashed section zoomed in (FIG. 13.B). The gene count ratio distribution is shown for HDTM Library 3 (FIG. 13.C) with the dashed section zoomed in (FIG. 13.D). All genes with a gene count ratio bellow the maximal value threshold were considered important in the given condition.

FIG. 14. pil genes are essential for transfer in vivo but not in vitro. The transposon insertion sites were mapped onto TP114 and visualized using the UCSC Genome Browser. Transposon insertion density is shown both for in vitro and in vivo conjugation experiments. Black lines represent insertion sites, and their height represent read density at a given position in TP114. Tracks shown represents HDTM Libraries 1, 3, 6 and 7 for biological replicate 2. While HDTM Library 1 shows the complete insertion profile for conjugation results in vitro, HDTM Library 3 shows insertion densities after two in vitro conjugation and HDTM Library 6 and 7 shows the effect of in vivo conjugation on the insertion densities. Comparison of the tracks clearly reveals a diminution in insertion signal intensity for the pil genes only for the two in vivo conjugation experiments (genes in dashed selection). Gene essentiality for in vivo conjugation is summarized in Table 10.

FIG. 15. Distribution of core and essential genes of TP114 for maintenance, in vitro conjugation and in vivo conjugation. Data for the conservation of genes and for gene essentiality as determined by HDTM were mapped onto TP114's sequence. Only essential genes with high confidence (black) and core genes (grey) are shown. Gene functions were attributed based on FIG. 7.

FIG. 16.A to E. Effect of T4P abolition on mating pair stabilization of TP114 in vitro. The pilS gene was deleted and complemented in T4P mutants of TP114 for conjugation under solid support, liquid static and agitating liquid conditions (FIG. 16.A). Briefly, ˜10⁸ CFUs of KN01 strain containing either TP114ΔpilS::cat or TP114ΔpilS::cat+ pPilS were mixed with an equal amount of the recipient strain KN03 to assess the importance of the T4P on TP114 conjugation efficiency. The resulting mixtures were incubated on solid medium or in liquid with and without shaking for 2 hours at 37° C. Conjugants were then resuspended (solid) or diluted (liquid) in 800 μL total volume, and plated on LB medium with appropriate antibiotics to evaluate the proportion of transconjugants in the entire recipient cell population. Error bars show standard deviation of the mean from at least 3 biological replicates. Asterisk indicates that frequency of transconjugant formation was below the limit of detection (<10⁻⁸). The entire pilV gene or the shufflon, which can re-organize the C-terminus of pilV (including the 3′-end of the pilV gene), was replaced by a Flag tag to generate a second set of T4P mutants. A plasmid allowing the expression of a pilV variant (TP114ΔpilV::cat+pPilV4′) was able to complement this phenotype using the same experimental conditions as described above (FIG. 16.B). Each possibility of locked pilV variant was assessed for conjugation under solid support, liquid static and agitating liquid conditions under the same conditions as described above (FIG. 16.C to E, respectively).

FIGS. 17.A to C. Effect of T4P inactivation on TP114 in vivo transfer rates. The ability of a TP114ΔpilS mutant to transfer in vivo was compared to TP114. Briefly, groups of 5 mice were treated with 1 g/L of streptomycin two days prior to strain introduction. Mice were administered the recipient strain KN03 2 hours prior to donor strain introduction. The proportion of transconjugants per recipient bacteria was then monitored in feces for four days (FIG. 17.A). On the fourth day, mice were sacrificed and the proportion of transconjugants was compared between the caecum and the feces (FIG. 17.B). Error bars show standard deviation of the mean from at least 5 biological replicates. A similar experiment was conducted for TP114ΔpilV::cat as well as two locked pilV variants (TP114Δshufflon::pilV1-cat and TP114Δshufflon::pilV4′-cat, which respectively failed and succeeded conjugation in vitro to a E. coli recipient strain), thus revealing the essential role of specific pilV variants for conjugation towards a given target bacterium (FIG. 17.C).

FIGS. 18.A to D. Incompatibility and exclusion hinder the transfer of conjugative plasmids. Incompatibility and exclusion mechanisms are specific to each Inc plasmid families. KN02 containing TP114 was used as a donor for conjugation towards recipient bacteria bearing different plasmids (pOX38, R6K, TP114::tetB, pVCR94, R388, RP4). TP114's transfer rate into a recipient bacterium devoid of any conjugative plasmid is shown by the dotted line, with standard deviation shown in gray (FIG. 18.A). Exclusion ratios were calculated based on the data of panel A. Briefly, the conjugation frequency of TP114 into a recipient devoid of any conjugative plasmid was divided by the transfer rate into a recipient already containing a plasmid. In trans mobilization of pCloDF13 by TP114 into a recipient containing or not a copy of TP114 was also assesed (FIG. 18.B). Exclusion was then tested in vivo. Streptomycin-treated mice fed with either KN02+TP114::tetB (SEQ ID NO: 166) or KN02 first, and subsequently fed with KN01+TP114 2 hours later. The proportion of transconjugants per recipient cells in feces was then analyzed daily for four consecutive days (FIG. 18.C). On the fourth day, mice were sacrificed and the proportion of transconjugants per recipient bacteria was compared between caecum and feces (FIG. 18.D). Error bars show standard deviation of the mean from at least 3 biological replicates.

FIGS. 19.A to C. Exclusion is abolished in specific TP114 mutants from HDTM Library 8. TP114::tetB (SEQ ID NO: 166) was transferred by conjugation into a mutant pool from HDTM Library 1. The resulting transconjugants were referred to as HDTM Library 8, and were mostly deficient for exclusion. Individual HDTM Library 8 mutants were isolated and then used as donor strain to isolate exclusion deficient clones of TP114::Tn5. After two successive rounds of conjugative transfers, the ability of TP114::Tn5 to exclude TP114::tetB (SEQ ID NO: 166) was verified in a conjugation experiment between KN02+TP114::tetB (SEQ ID NO: 166) and KN01, KN01+TP114 or KN01+TP114::Tn5 as a recipient (FIG. 19.A). The exclusion ratio of TP114 or TP114::Tn5 was also compared to an empty recipient as previously described in FIG. 18 (FIG. 19.B). Finally, the capacity of a TP114::Tn5 mutant to transfer at expected rates was verified on solid medium for 2 hours at 37° C. using KN01 as the donor cell and KN03 as the recipient (FIG. 19.C). Error bars show standard deviation of the mean from at least 3 biological replicates.

FIG. 20. Genes limiting TP114's transfer efficiency. Transposon insertion sites were aligned onto TP114 and visualized using the UCSC Genome Browser. Representative insertion density tracks for in vitro (HDTM Library 3) and in vivo (HDTM Library 7) conditions are shown. Vertical black lines represents transposon density at a given position of the TP114 genome. The tracks shown represent HDTM Library step 1, 3 and 7 for biological replicate #2 as presented in FIG. 9. Only two genes showed enrichments from HDTM Library 1 to HDTM Libraries 3 and 7: TP114-005 (previously shown to mediate exclusion and a gain in conjugation frequency) and yaeC (a homolog of transcription repressor fin 0). Both genes are boxed in a dotted frame.

FIGS. 21.A to G. Plasmids and gRNAs used to test cargos Kill1 and Kill3. Maps of the Kill1 insertion device (FIG. 21.A), Kill3 insertion device (FIG. 21.B), pREC1 (SEQ ID NO: 160) (FIG. 21.C), pBXB1 (SEQ ID NO: 145) (FIG. 21.D) and pT (FIG. 21.E) are shown to scale. Total length in base pair (bp) is displayed bellow the plasmid name. gRNAs (an engineered fusion of the tracrRNA and the crRNA) from Kill3 are designed with the same promoters and terminators as Kill1's gRNA. The asterisks in the cat gene in pT's map represent the protospacer of the gRNAs. All gRNAs were designed to target cat, a chloramphenicol resistance gene, which was introduced in the target's genome or present on a plasmid. The gRNA's spacers sequences match the target sequence in the cat gene (FIG. 21.F). The complete nucleotide sequence of the cat gene (SEQ ID NO: 87) shows the location of gRNA 1 (SEQ ID NO: 88, light gray), gRNA 2 (SEQ ID NO: 89, gray) and gRNA 3 (SEQ ID NO: 90, dark gray) protospacers and their protospacer-associated motif (PAM) (framed with a solid line) (FIG. 21.G).

FIGS. 22.A to D. Introduction of a genetic cargo in the transfer machinery by Double Recombinase Operated Insertion of DNA (DROID). The DROID method is exemplified by the insertion of Kill1 in the transfer machinery TP114 (FIG. 22.A). The first step is to insert the tetB loading dock in the transfer machinery by recombineering. Then, the Bxb1 integrase operates the fusion between the attB and attP sites located on the loading dock and on the genetic cargo insertion device respectively. Lastly, a FLP recombinase is expressed to knock out the insertion device segment between the two newly joined FRT sites (tetB, pSC101ts and the attL site). PCR verifications for the recombineering (FIG. 22.B), the DROID step 1 (FIG. 22.C) and the DROID step 2 (FIG. 22.D) are shown for Kill1's insertion. Amplicons of each lanes are identified on the drawings in bold letters or numbers except for lane D which is the right junction between tetB and TP114 in TP114::tetB.

FIGS. 23.A and B. Examples of conjugative delivery system configurations. The bacterium can be decomposed into several components organized hierarchically (FIG. 23.A). The genetic cargo can be delivered in several configurations, three of which are shown in example III (FIG. 23.B). The first approach is to deliver a genetic cargo by cis mobilization, where the genetic cargo is directly inserted in the transfer machinery to form a single vector encoding the Conjugative Delivery System. A second method is to deliver the genetic cargo through constrained cis mobilization, where the essential replication genes are relocated in the chromosome of the donor bacterium to prevent replication of the Conjugative Delivery System outside the donor strain. Finally, the genetic cargo and Transfer machinery can be encoded on two or more vectors to allows in trans mobilization. In this set-up, the genetic cargo needs a transport module which is recognized by the transfer machinery and mediates its transfer from the donor strain to the recipient strain. Each delivery mode presents different levels of biosafety, which are represented by an X (not biocontained), a + (contained) and a ++ (more strictly contained). Replication and transfer capacity in both the donor and the recipient strains are shown by an X (not possible) or check marks (possible). Replication in the recipient for in trans mobilization is dependent on the maintenance module of the genetic cargo.

FIG. 24. Transformation efficiencies of Kill1 and Kill3 genetic cargos assessed by transformation into a recipient cell harboring a target plasmid (pT). 50 ng of each genetic cargo insertion device were electroporated in biological triplicates into KN03+pT and plated to select only the genetic cargo (black bars) or to select both the genetic cargo and pT (white bars). Transformation efficiencies are shown as transformants per mg of electroporated DNA. Error bars show standard deviation of the mean from at least 3 biological replicates. Asterisk indicates the absence of CFUs on plate.

FIGS. 25.A and 25.B. TP114::Kill1 to selectively eliminates a target bacterium from a mixed population in vitro. The COP is used for the specific targeting of an E. coli Nissle 1917 (FIG. 25.A) or Citrobacter rodentium (FIG. 25.B) carrying a chromosomal copy of the cat gene and this, in a mock bacterial population composed of three other Enterobacteriaceae. Equal amounts of each strain were mixed, and then incubated with the COP strain, or with KN01ΔdapA+TP114 control strain, for 2 hours on solid medium at 37° C. The graphic shows the relative abundance (%) of transconjugants for TP114::Kill1 as compared with TP114 for each strain of the mock population. In both cases, the abundance of the targeted strain transconjugants was specifically decreased by ˜1000 fold. Error bars show standard deviation of the mean from at least 3 biological replicates.

FIG. 26. Identification of the TP114 origin of replication (oriV) locus. The locus encoding the replication protein RepA was predicted in silico, and cloned in three different configurations in a pir-dependent plasmid backbone. “repA+up” corresponds to the repA coding sequence (CDS)+1,000 bp from the upstream region; “repA+down” represents the repA CDS and putative promoter+1,000 bp in the downstream region; “repA+both” encompasses the repA CDS+1,000 bp from the upstream and downstream regions. All three plasmid versions were transformed in a pir− or pir+ strain to test the activity of TP114 oriV. Only the repA CDS with both upstream and downstream regions could replicate in a pir− strain. Error bars show the standard deviation of the mean from 3 biological replicates.

FIGS. 27.A and 27.B. Constrained cis mobilization delivery can prevent plasmid persistence in the environment. TP114 and TP114ΔrepA::cat-oriV_{R6K} were transferred by conjugation from EC100Dpir+ towards a pir-devoid strain (black bars) and a pir+ strain (white bars) at 37° C. on solid LB medium for 2 hours. No transconjugant was detected for TP114ΔrepA::cat-oriV_{R6K} towards the pir-devoid strain (asterisk) and reduced frequency was observed for TP114ΔrepA::cat-oriV_{R6K} towards the pir+ strain. In stark contrast, wild-type TP114 could transfer at normal frequency (˜10⁻³) (FIG. 27.A). TP114 and TP114ΔrepA::cat-oriV_{R6K} were transferred by conjugation from KN05 towards a EC100Dpir+ strain (gray bars) at 37° C. on solid LB medium for 2 hours (FIG. 27.B). Under these conditions, both plasmids could conjugate at similar rates. Error bars show the standard deviation of the mean from 3 biological replicates.

FIGS. 28.A and 28.B. In trans mobilization of oriT_{TP114}-containing plasmids. Shuttle plasmid pNA01 contains oriT_{TP114} and therefore should be mobilizable by TP114. pNA02 presents a 7-bp deletion centered on the nicking site of oriT_{TP114}. In trans mobilization frequencies for plasmids pNA01 (FIG. 28.A) and pNA02 (FIG. 28.B) using a donor strain containing TP114 and a shuttle plasmid (either pNA01 or pNA02). Conjugation frequencies were calculated for transconjugants containing TP114 (black bars), the shuttle plasmid pNA01 or pNA02 (gray bars) and for transconjugants harbouring both TP114 and a shuttle plasmid (white bars). Error bars show the standard deviation of the mean from 3 biological replicates.

FIG. 29. Localization of TP114's origin of transfer (oriT) nicking site by pairwise sequence alignment. The oriT allows the recognition of a plasmid and is essential for mobilization. This recognition is based on the presence of repeats within the oriT sequence. The relaxosome then specifically binds the oriT and nicks (single strand break) the DNA to initiate conjugative transfer. TP114's oriT (SEQ ID NO: 141) was aligned with previously characterized R64 minimal oriT (SEQ ID NO: 142). Important repeats were mapped onto the alignment to allow for prediction of the nicking site. While sequence alignment was weak, repeats were present both in TP114 and R64, suggesting the putative localization of the nicking site. Lines ‘|’ represents a perfect sequence alignment, dots shows low similarity regions and a blank space ‘ ’ is a gap of a mismatch.

FIGS. 30.A and 30.B. Impact of the deletion of the origin of transfer (oriT) on TP114 conjugation frequency. The approximate location of the nicking site in TP114's oriT was deleted by recombineering, creating TP114ΔoriT. Conjugation frequency was first evaluated using transfers from E. coli MG1655Nx^{R} into E. coli MG1655Rf^{R} and compared to wild-type conjugation rates (FIG. 30.A). The transfer rate was drastically reduced in TP114ΔoriT, with residual transfer events (˜10⁻⁶) likely due to partial oriT recognition or by the presence of a cryptic oriT sequence in TP114. Transfer of TP114ΔoriT was then tested towards E. coli MG1655Rf^{R} and, no transconjugants were detectable (asterisk) (FIG. 30.B). Error bars show the standard deviation of the mean from 3 biological replicates.

FIG. 31. In trans-mobilization of shuttle plasmids pKN30 and pKN31 by a non-mobilizable transfer machinery. In trans mobilization frequencies for plasmids pKN30 (FIG. 28.A) and pKN31 (FIG. 28.B) using a donor strain containing both TP114 and a shuttle plasmid (either pKN30 or pKN31). Plasmids pKN30 and pKN31 are kanamycin resistant variants of pNA01 and pNA02, respectively. Both pKN30 and pKN31 contain TP114's oriT, but pKN31 has a 7-bp deletion in the nicking site region to prevent its transfer. Conjugation frequencies were calculated for transconjugants containing TP114ΔoriT::cat-tetB, the shuttle plasmid (pKN30 or pKN31) and for transconjugants harbouring both TP114 and a shuttle plasmid. Asterisk indicates that frequency of transconjugant formation was below the limit of detection (<10⁻⁸). Error bars show the standard deviation of the mean from 3 biological replicates.

FIG. 32. Schematic representation of in trans mobilization as described in Example III. The TP114 oriT sequence was identified in silico and cloned into pNA01. The oriT sequence is recognized by the TP114 nickase to mediate pNA01 in trans mobilization. However, a 7-bp deletion centered on the predicted nicking site (essential for nickase activity) prevents in trans mobilization of pNA02.

FIGS. 33.A and 33.B. The COP system can transfer DNA conferring a beneficial phenotype to a target bacterium in vivo. TP114 was used as a Conjugative Delivery System to transfer the kanamycin resistance gene to a target bacteriumin the gut of mice. Mice were fed with KN02 two hours prior to KN01+TP114 introduction. Proportion of recipient bacteria that have acquired the resistance phenotype (transconjugants) relative to total recipients was followed for 4 days in feces (FIG. 33.A). On the fourth day, mice were sacrificed and the proportion of transconjugants per recipients was compared in the caecum and in the feces (FIG. 33.B). Error bars show the standard deviation of the mean from 4 biological replicates.

FIGS. 34.A to D. Application of the conjugative probiotic (COP) as presented in the Example IV. The COP system is based on a probiotic cellular chassis delivering a genetic cargo by conjugative transfer. In the present example, the genetic cargo encodes CRISPR-Cas9 which can be transferred to a population of bacteria and target specific strains for elimination based on sequence specific criteria (FIG. 34.A). Conjugation is mediated by the transfer machinery encoded by a highly efficient conjugative plasmid, which in this example, directly harbours the genetic cargo hereby forming the conjugative delivery system. Conjugative plasmids are also usually modular, with genes grouped according to their function (FIG. 34.B). Once in the target cell, Cas9 endonuclease is expressed and assembles with the gRNA and scans the entire DNA content of the cell. Once a protospacer sequence is found downstream of a protospacer-adjacent motif (PAM) sequence, Cas9 mediates the double stranded cleavage of the DNA (FIG. 34.C). The bacterial COP can be used to selectively target cells in a complex microbial community. The bacterial COP will transfer the genetic cargo to recipient cells; however, the Cas9-gRNA system will only target specific strains from the community. If the target sequence is genomic, the target cells will die from DNA compromised genome integrity; if the target is plasmidic (e.g., virulence associated gene), the plasmid will be cured leading to target cell disarmament (FIG. 34.D).

FIGS. 35.A to D. COP can mediate loss of phenotypic traits through CRISPR-Cas9 extra-chromosomal sequence targeting. TP114 (control) or TP114::Kill1 was transferred from KN01 to KN02 (harbouring the target plasmid pT) within the mouse intestinal tract. Target strain disarming (pT plasmid curation) was followed through time in feces for 4 days by analyzing colony fluorescence on plate allowing growth of all target recipient cells (FIG. 35.A). One-way ANOVA was performed on the raw percentage of the four mice from the control group and three of the test group mice in response to the COP treatment. A representative image shows how green fluorescence was discernable between the colonies that had lost the plasmid and those who did not (FIG. 35.B). On the fourth day, plasmid curation results were compared in the feces and in the caecum of the mice and showed high consistency (FIG. 35.C). When selecting for transconjugants only, TP114::Kill1 achieved 100% disarming rates in all mice, but TP114 transfer showed no such activity (FIG. 35.D).

FIGS. 36.A to E. COPs can be administered prophylactically to prevent colonization by an invading strain in vivo. Schematic description of the experiment (FIG. 36.A). A probiotic donor strain bearing the TP114 plasmid with or without the CRISPR-Cas9 system (COP and control, respectively) was administered (˜10⁸ CFUs) 12 hours prior to the target/non-target strain mix. The abundance (FIGS. 36.B and D) and competitive index (FIGS. 36.C and E) of the target and non-target strains per mg of feces are presented as a function of time after gavage of the donor strain. The competitive index shows the relative abundance of the target or non-target bacteria. The dotted line in panel B and D is the upper limit for detection of CFU while the y axis is set to the lower limit of detection. In panel C and E, the dotted line represent a competitive ratio equivalent for both strains, in a situation where both strains would have exactly the same fitness.

FIGS. 37.A to E. COPs can be administered therapeutically to selectively eliminate a target strain in vivo. Schematic description of the experiment (FIG. 37.A). The target/non-target strain mix was administered (˜10⁸ CFUs) 12 hours prior to the probiotic donor strain bearing the TP114 plasmid with or without the CRISPR-Cas9 system (COP and control, respectively). The abundance (FIGS. 37.B and D) and competitive index (FIGS. 37.C and E) of the target and non-target strains per mg of feces are presented as a function of time after gavage of the target and non-target strain mix. The competitive index shows the relative abundance of the target or non-target bacteria. The dotted line in panel B and D is the upper limit for detection of CFU while the y axis is set to the lower limit of detection. In panel C and E, the dotted line represent a competitive ratio equivalent for both strains, in a situation where both strain would have exactly the same fitness.

FIGS. 38. A to C. The genetic cargo can generate beneficial and detrimental effects on bacterial populations. In this experiment, TP114::Kill3 encoded a kanamycin resistance gene and a CRISPR-Cas9 system targeting the gene responsible for chloramphenicol resistance. Both TP114::Kill3 and TP114 (control) were transferred by conjugation in a recipient bacterium bearing a target plasmid (pT). Plasmid curation efficiency was first monitored through antibiotics resistance where co-existence of the plasmid pT with TP114 or TP114::Kill3 was assessed. Co-selection of pT and TP114::Kill3 led to a clear drop in CFU which suggests that selection of the genetic cargo would lead to higher pT loss within the recipient bacteria population. Asterisk indicates that frequency of transconjugant formation was below the limit of detection (<10⁻⁸) (FIG. 38.A). Plasmid curation was then evaluated by quantifying the number of fluorescent colonies (containing the pT plasmid) while selecting the acquisition of the genetic cargo using kanamycin (FIG. 38.B). A representative picture of bacterial spots from serially-diluted conjugative mixtures after growth of target cells with kanamycin selection (FIG. 38.C). Error bars show the standard deviation of the mean from 3 biological replicates.

FIGS. 39.A and B. Conjugative transfer rates of several plasmids between the EcN donor and different recipient strains from various bacterial species. In vitro transfer of several conjugative plasmids spanning six incompatibility families towards strains representing some of the most infamous multidrug resistant Enterobacteriaceae species. Transfer experiments were performed both on agar (FIG. 39.A) and in broth (FIG. 39.B). Transfer frequency normalized on recipient CFUs is represented using a grayscale gradient. Data shown are the average of at least 3 biological replicates.

FIG. 40. Protection from restriction systems can be acquired through DNA modification. Restriction modification systems are a barrier to horizontal gene transfer. Using donor that possess a modification system compatible with the recipient's restriction system improves the conjugative efficiency. In the legend, MG1655=E. coli MG1655 and EcN=E. coli Nissle 1917. Transfer is done between the donor-recipient couple. Data shown are the average of at least 3 biological replicates.

### DETAILED DESCRIPTION

The present disclosure relates to the methods and systems for developing and using a conjugative bacterial cell specifically engineered to deliver a payload, such as a therapeutic genetic cargo, in vivo to a recipient bacterial host cell. In a specific embodiment, the conjugative bacterial cell can be used in vivo (e.g., in the gastro-intestinal tract environment or in the bladder, for example). In some embodiments, the conjugative bacterial cell can be used to (1) treat microbiota dysbiosis, (2) modify a microbiota to express beneficial factors, (3) suppress antibiotic resistance and/or the spread of antibiotic resistance, (4) eliminate a specific pathogen, and (5) suppress the expression of bacterial virulence factors.

As used in the context of the present disclosure, the term “derived” refers to the use of genetic material that has been obtained or modified from a naturally-occurring organism.

Components of the Conjugative Delivery System

The conjugative delivery system of the present disclosure comprises genetic elements present natively or genetically introduced in a bacterium allowing the bacterium to transfer in vivo its genetic cargo to a recipient bacterial cell. The conjugative delivery system comprises two main components: a transfer machinery (which includes the genetic elements required to transfer the genetic cargo) and the genetic cargo itself. The components of the transfer machinery can be located on one or more extrachromosomal vector and/or integrated in the bacterial's chromosome. The components of the transfer machinery can be located in cis or in trans with respect to each other. The genetic cargo has been genetically engineered in the conjugative bacterial host cell either by positioning a transport module in operative association with the payload module or by introducing an heterologous genetic cargo in the conjugative bacterial host cell. The genetic cargo which includes a transport module operatively associated with a payload module can be located on an extrachromosomal vector or integrated in the bacterial's chromosome. The transport module is “operatively associated” with the payload module which allows the transfer of the payload module when the proteins encoded by the mobilization module (e.g., the transport machinery) recognize and act upon the transport module. Therefore, on the genetic cargo, the payload module is located in cis to the transport module at a position allowing the transfer of the payload module when the proteins of the mobilization module associate with the transport module.

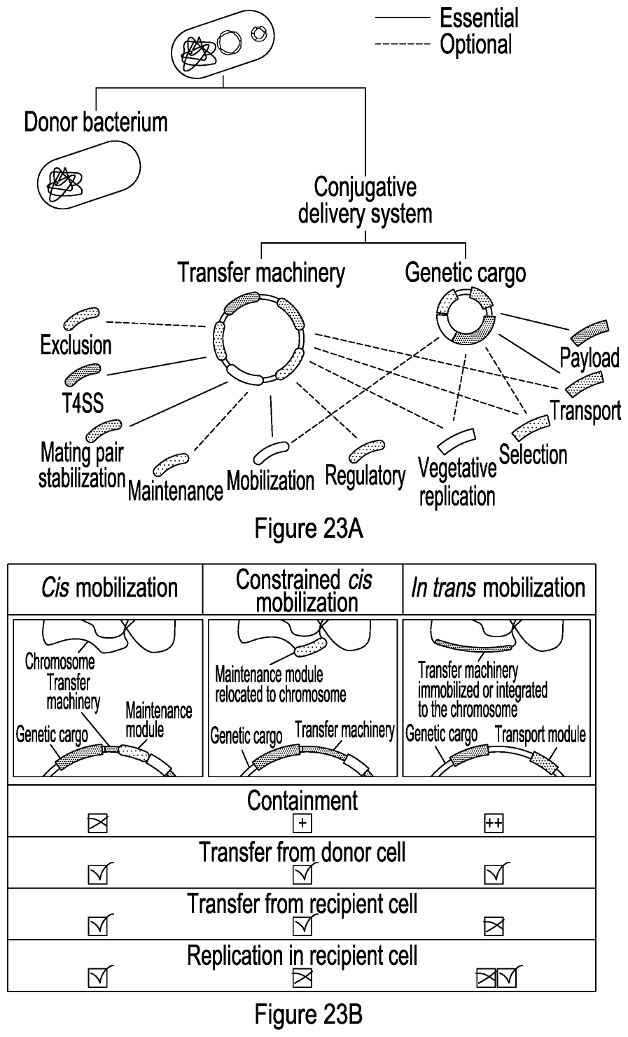

In an embodiment, the components of the transfer machinery and of the genetic cargo are exclusively located on one or more extrachromosomal vector. In a specific embodiment, the components of the transfer machinery and of the genetic cargo are located on a single extrachromosomal vector. In another embodiment, the components of the transfer machinery and of the genetic cargo are located on more than one extrachromosomal vectors. For example, the components of the transfer machinery and of the genetic cargo can be organized in two distinct chromosomal vectors as shown in FIG. 23A.

In another embodiment, the components of the transfer machinery and of the genetic cargo are/can be located exclusively in the bacterial's chromosome.

In yet a further embodiment, the components of the transfer machinery and of the genetic cargo are/can be located on one or more extrachromosomal vectors as well as in the bacterial's chromosome. For example, the components of the transfer machinery can be located exclusively in the bacterial chromosome and the components of the genetic cargo can be located exclusively in an extrachromosomal vector. In another example, some of the components of the transfer machinery can be located in the bacterial chromosome as well as in one or more extrachromosomal vectors while the components of the genetic cargo can be located exclusively in one or more extrachromosomal vector (such as, for example, the embodiments shown in FIG. 23B). In still another example, the components of the transfer machinery can be located exclusively in the bacterial chromosome while some of the components of the genetic cargo can be located in the bacterial chromosome and others can be located in one or more extrachromosomal vector. In still a further example, some of the components of the transfer machinery can be located in the bacterial chromosome as well as in one or more extrachromomal vectors while some of the components of the genetic cargo can be located in the bacterial chromosome and others can be located in one or more extrachromosomal vector.

As used herein, the term “genome” refers to the whole hereditary information of an organism that is encoded in the DNA including both coding and non-coding sequences. The term “module” refers to a group of genes that contribute to a same function. In an embodiment, all genes from a same module are physically linked (in cis) on the same DNA molecule. In yet another embodiment, the genes can be contained on more than one DNA molecule.

As used herein, the term “extrachromosomal vector” refers to a genetic element which is physically distinct from the bacterial genome. The extrachromosomal vector is usually capable of independent replication from the bacterial genome due to the presence of a vegetative replication module. In some embodiments, the extrachromosomal vector is a plasmid, such as, for example, a circular plasmid. Vectors can be circular plasmids, usually when it is intended that the vector is independently replicating from the genome of the donor bacterium, or vectors can be linear DNA molecules integrated in the genome of the donor bacterium. In embodiments in which more than one vector is present, they can be provided in the same or different forms.

In an embodiment the transfer machinery and the genetic cargo can be part of the same nucleic acid molecule or different nucleic acid molecules. The nucleic acid molecules can be circular or linearized (and intended for integration in the bacterial's chromosome).

The transfer machinery and the genetic cargo include modules comprising genes which can encode one or more proteins, variants thereof or fragments thereof. The protein can be a variant of a a protein known to be encoded by the module. A variant comprises at least one amino acid difference when compared to the amino acid sequence of the native/known protein. As used herein, a variant refers to alterations in the amino acid sequence that do not adversely affect the biological functions of the protein. A substitution, insertion or deletion is said to adversely affect the protein when the altered sequence prevents or disrupts a biological function associated with the heterologous protein. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the heterologous protein. The protein variants have at least 50%, 55%, 60%, 65%, 70%, 75%, 80%, 85%, 90%, 95%, 96%, 97%, 98% or 99% identity to the heterologous protein described herein. The term “percent identity”, as known in the art, is a relationship between two or more protein sequences or two or more nucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant protein described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the protein can be a conservative variant or an allelic variant.

The protein can be a fragment of a protein encoded by one of the genes of the module or a fragment of a variant protein. In an embodiment, the fragment corresponds to the known/native protein to which the signal peptide sequence has been removed. In some embodiments, heterologous protein “fragments” have at least at least 100, 200, 300, 400, 500, 600, 700, 800, 900 or more consecutive amino acids of the protein. A fragment comprises at least one less amino acid residue when compared to the amino acid sequence of the known/native heterologous protein and still possess the enzymatic activity of the full-length heterologous protein. In an embodiment, the fragment corresponds to the amino acid sequence of the protein lacking the signal peptide. In some embodiments, fragments of the heterologous protein can be employed for producing the corresponding full-length heterologous by peptide synthesis. Therefore, the fragments can be employed as intermediates for producing the full-length proteins.

Conjugative Bacterial Cell

As used herein, the terms “conjugative bacterial (host) cell”, “recombinant bacterium” or “donor bacterium” refer to a bacterium capable of horizontal gene transfer via bacterial conjugation. As used in the context of the present disclosure the terms “bacterial conjugation”, “conjugation”, “conjugative transfer” or “transfer” refer to a mechanism of horizontal gene transfer where genetic material (referred to as the genetic cargo) is delivered from a donor bacterium to a target bacterium (also referred to as a recipient bacterial cell) through a conjugative pore forming a channel between the two bacterial cells. The conjugative bacterial cell can be, in some embodiments, modified prior to being used in conjugation so as to remove or inactivate one or more virulence factors. In some embodiments, the conjugative bacterial cell can be a probiotic bacterium which can be referred to as a “conjugative probiotic” or “COP”. As used herein, the term “probiotic” refers to a bacterium that, once administered in adequate amount and via adequate routes, has no detrimental effects and may also provide beneficial effects to its host.

The present disclosure thus provides a bacterium, which can be a probiotic, which has been genetically engineered to bear the conjugative delivery system of the present disclosure. Thus, the present disclosure also provides a process for obtaining the recombinant bacterium by introducing the conjugative delivery system of the present disclosure in a bacterium.

Bacterial genera referred to as probiotic to a human or animal subject and that could be the COP of the present disclosure include, but are not limited to, Bacillus sp., Bifidobacterium sp., Enterococcus sp., Escherichia sp., Lactobacillus sp., Lactococcus sp., Leuconostoc sp., Pediococcus sp. and Streptococcus sp. As such, the present disclosure provides a probiotic recombinant bacterium from the genus Bacillus sp., Bifidobacterium sp., Enterococcus sp., Escherichia sp., Lactobacillus sp., Lactococcus sp., Leuconostoc sp., Pediococcus sp. and Streptococcus sp. as well as a process for making such probiotic bacteria by introducing the conjugative bacterial vector or system in a probiotic bacterium of the genus Bacillus sp., Bifidobacterium sp., Enterococcus sp., Escherichia sp., Lactobacillus sp., Lactococcus sp., Leuconostoc sp., Pediococcus sp. or Streptococcus sp. Bacterial species which are considered probiotic to human subjects include, but are not limited to Bacillus coagulans (e.g., strain GBI-30 or 6086), Bifidobacterium animalis subsp. lactis (e.g., strain BB-12), Bifidobacterium longum subsp. infantis, Enterococcus durans (e.g. strain LAB18s), Escherichia coli (e.g., strain Nissle 1917), Lactobacillus acidophilus (e e.g., strain NCFM), Lactobacillus bifidus, Lactobacillus johnsonii (e.g., strain Lai, LCI or NCC533), Lactobacillus paracasei (e.g., strain Stl 1 or NCC2461), Lactobacillus plantarum (e.g., strain 299v), Lactobacillus reuteri (e.g., strain ATCC 55730, SD2112, Protectis, DSM 17938, Prodentis, DSM 17938, ATCC 55730, ATCC PTA 5289, RC-14), Lactobacillus rhamnosus (e.g., strain GG, GR-1) and Lactococcus thermophiles, Leuconostoc masenteroides (e.g. strain B7), Pediococcus acidilactici (e.g. strain UL5), and Streptococcus thermophilus. As such, the present disclosure provides a probiotic recombinant bacterium from the bacterial species which are considered probiotic to human subjects as well as a process for making such probiotic bacteria by introducing the conjugative bacterial vector or system in a probiotic bacterium of the strains of bacteria considered probiotic in humans. In a specific embodiment, the probiotic is from the genus Escherichia, for example the species Escherichia coli, e.g. E. coli Nissle 1917. The present disclosure provides a process for making such probiotic recombinant bacteria by introducing the conjugative bacterial vector or system in a probiotic species Escherichia coli, e.g. E. coli Nissle 1917.

Transfer Machinery

The conjugative bacterial host cell comprises a genetic cargo, a type IV secretion system module, a mobilization module and a mating pair stabilization module comprising a type IV adhesion pilus, the type IV adhesion pilus comprising an adhesin. In some embodiments, the modules that are not part of the genetic cargo can be organized into the transfer machinery.

The transfer machinery is responsible for allowing the formation of a conjugative pore and the subsequent physical transfer of the genetic cargo into the recipient bacterium. The transfer machinery includes genes and regulatory elements that are divided in different modules further described below. The genes present within those modules can optionally be organized in the form of one or more operons.

As used herein, the term “gene” refers to a nucleic acid molecule containing the sequence information necessary for expression of a protein or a non-coding RNA (e.g. tracrRNA, crRNA, gRNA, rRNA, tRNA, anti-sense RNA). When the gene encodes a protein, it includes the promoter and the structural gene open reading frame sequence (ORF), as well as other sequences involved in the expression of the protein. When the gene encodes a non-coding RNA, it includes the promoter and the nucleic acid that encodes the untranslated RNA. As indicated above, genes may be expressed in the form of one or more operons. As used herein, the term “operon”, as it is known in the art, is a functional unit containing a cluster of genes under the control of a single promoter.

The term regulatory element refers to promoters, activator/repressor binding sites, terminators, enhancers and the like. In an embodiment, more than one promoter is included in the bacterial conjugative delivery system of the present disclosure. In yet another embodiment, only one promoter is included in the conjugative delivery system of the present disclosure.

When present, a promoter can be constitutive or inducible. The terms “constitutive” and “inducible” refer to the dynamic state of expression. A constitutive expression is stable overtime whereas an inducible expression allows a significant change in the level of expression of a gene. An inducible expression can be achieved in various ways such as the activation of transcription by a transcription activator, the repression of transcription by a transcription repressor or the control of translation by a functional 5′ untranslated region commonly referred to as a riboswitch.

In an embodiment, the transfer machinery comprises a type IV secretion system (T4SS) module, a mating pair stabilization module and a mobilization module. In some embodiments, the transfer machinery can optionally comprise a transport module, a regulatory module, a vegetative replication module, a maintenance module, a selection module and/or an exclusion module.

The T4SS module includes genes and regulatory elements responsible for the formation of a type IV secretion system. The T4SS is a protein assembly capable of establishing a conjugation pore that forms a channel between the donor bacterium and the recipient bacterium. It is through this conjugation pore that the genetic cargo is transferred from the donor bacterium to the recipient bacterium. In some embodiments, the T4SS module (which can be heterologous to the conjugative bacterial cell) is integrated in the genome of the conjugative bacterial cell. In another embodiment, the T4SS module is located in one or more extrachromosomal vectors (such as plasmids) which may be endogenous or heterologous to the conjugative bacterial cell. The genes present in the T4SS module include, but are not limited to, one or more of virB1 (TP114-012: traB), virB2 (TP114-013: traC), virB3 (TP114-014: traD), virB4 (TP114-015: traE), virB5 (TP114-004: trbJ), virB6 (TP114-003: traA), virB7 (TP114-011: ygeA), virB8 (TP114-017: traG), virB9 (TP114-018: traH), virB10 (TP114-019: traI), virB11 (TP114-020: traJ) and/or virD4 (TP114-021: traK). As such, the T4SS module can include one or more genes encoding one or more proteins of a T4SS. In addition, one or more T4SS conjugative pore, as well as, one or more different types of T4SS can be encoded by the T4SS module and expressed by the donor bacterium. In an embodiment, the genes encoding the T4SS can be derived from one or more of the following family of bacterial conjugative plasmids MPF_{T}, MPF_{F}, MPF_{I}, MPF_{FATA}, MPF_{B}, MPF_{FA}, MPF_{G} and/or MPF_{C}. In another embodiment, the genes encoding the T4SS can be derived from one of the MPF_{T} family of bacterial conjugative plasmids. For example, the genes encoding the T4SS can be derived from the bacterial plasmid TP114. In another example, the genes encoding the T4SS can be derived from the bacterial plasmid R6K. In yet another embodiment, the genes encoding the T4SS can be derived from one of the MPF_{F} family of conjugative plasmids. In yet another embodiment, the genes encoding the T4SS can be derived from the bacterial vector F (or pOX38).

The transfer machinery also includes a mating pair stabilization module. The mating pair stabilization module includes genes and regulatory elements responsible for the stabilization of the physical interaction of the donor bacterium with the target bacterium. As shown in Example II below, stabilizing the interaction between the donor bacterium and the target bacterium favors maintaining a physical proximity necessary for the establishment of the T4SS conjugative pore, which is important for the subsequent transfer of the genetic cargo in an unstable environment (in vivo or liquid for example). The stabilization of the interaction between the donor bacterium and the target bacterium is particularly important in vivo (e.g., in the gastro-intestinal environment or the bladder) where perturbations could affect transfer from the conjugative bacterial cell to target bacterium. The mating pair stabilization module (which may be heterologous to the conjugative bacterial cell) can be integrated in the genome of the conjugative bacterial cell or located on one or more bacterial vectors.

The mating pair stabilization module includes genes and regulatory elements responsible for the formation of a type IV adhesion pilus. The mating pair stabilization module (which may be heterologous to the conjugative bacterial cell) can be integrated in the genome of the conjugative bacterial cell or located on one or more bacterial vectors. Type IV adhesion pilus, as used herein, are protein assemblies forming long thin filaments that protrude from, and retract into, bacterial cells. The presence of type IV adhesion pilus on the membrane of a donor bacterium is believed to facilitate the “capture” of a target bacterium by physically “grabbing” it and “pulling” it. The presence of type IV adhesion pilus on the membrane of a donor bacterium thus stabilizes the interaction of the donor bacterium with the target bacterium. Type IV adhesion pilus genes include, but are not limited to one or more of pilL (TP114-009), pilN (TP114-022), pilO (TP114-023), pilP (TP114-024), pilQ (TP114-025), pilR (TP114-026), pilS (TP114-027), pilT (TP114-028), traN, traB (TP114-012), pilU (TP114-029) and/or pilV (TP114-030). As such, the mating pair stabilization module can include one or more genes encoding one or more proteins of a type IV adhesion pilus. In addition, one or more type IV adhesion pilus, as well as, one or more different types of type IV adhesion pilus can be encoded by the mating pair stabilization module and expressed by the donor bacterium. In an embodiment, the genes encoding the type IV adhesion pilus can be derived from one of the following family of bacterial conjugative plasmids IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14, Inc18. In another embodiment, the genes encoding the type IV adhesion pilus can be derived from one of the incompatibility family of bacterial conjugative plasmids capable belonging to the I-complex: IncI1, IncI2, Incly, IncB/O (Inc10), IncK and/or IncZ. In another embodiment, the genes encoding the type IV adhesion pilus can be derived from one of the IncI2 family of bacterial conjugative plasmids. For example, the genes encoding the the type IV adhesion pilus can be derived from the bacterial vector TP114.

The type IV adhesion pilus comprises an adhesin. Adhesin are proteins which can, when displayed on the surface of a donor bacterium membrane, interact with various molecules present on the outer membrane of a target bacterium (e.g., proteins, sugars, lipids). For example, the PilV adhesin from the IncI2 family of bacterial conjugative plasmids interacts with receptors such as lipopolysaccharides (LPS), which are molecules typically found on the outer membrane of Gram-negative bacteria. Hence, if a donor bacterium displays a PilV adhesin on its outer membrane, PilV will bind to the LPS of a Gram-negative target bacterium and stabilize the interaction of the two cells. Adhesins include, but are not limited to, one or more of pilV (TP114-030) from TP114, pilV from R64, traN from pOX38. In some embodiments, one or more adhesin, as well as, one or more different types of adhesin can be encoded by the mating pair stabilization module and expressed by the donor bacterium. In an embodiment, adhesins can be displayed on the surface of the donor bacterium by either being part of an accessory pili protein assembly (e.g., like type IV adhesion pilus), and/or by being part of a T4SS conjugative pili protein assembly, and/or by being part of any molecular complex allowing the adhesin to be displayed on the surface of the bacterium. In another embodiment, the genes encoding adhesins can be derived from one of the following family of bacterial conjugative plasmids IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Mot Inc4, Inc7, Inc8, Inc9, Inc11, Inc13, Inc14, Inc18. In another embodiment, the genes encoding the type IV adhesion pilus can be derived from one of the incompatibility family of bacterial conjugative plasmids capable belonging to the !-complex: IncI1, IncI2, Incly, IncB/O (Inc10), IncK and/or IncZ. In yet another embodiment, the genes encoding the adhesins can be derived from one of the IncI2 family of bacterial conjugative plasmids. For example, the genes encoding adhesins can be derived from the bacterial vector TP114. In yet another embodiment, the genes encoding the adhesins can be derived from one of the IncFII family of bacterial conjugative plasmids. For example, the genes encoding adhesins can be derived from the bacterial vector pOX38. In yet another embodiment, the genes encoding the adhesins can be derived from one of the IncX family of bacterial conjugative plasmids. For example, the genes encoding adhesins can be derived from the bacterial vector pR6K.

Adhesin genes can optionally be rearranged by the presence of shufflons and the activity of a shufflase. A shufflon is a cluster of multiple DNA inversions segments which can be located in the 3′ end of an adhesin gene. Under the action of a shufflase, an enzyme with a recombinase activity, the sequential order of different segments of the shufflon can be randomly rearranged. Following this rearrangement, the one segment that aligns with the adhesin gene becomes the end of the adhesin gene. Therefore, when an adhesin gene is associated with a shufflon, the distal section of the gene is variable and can potentially be any of the different DNA inversions segments included in the shufflon. As a result, when an adhesin gene with a shufflon is transcribed and translated, the C-terminus end of the adhesin is also variable and corresponds to a shufflon's inversion segment that ends the adhesin gene. Each shufflon's segment confers specific binding affinities to the adhesin protein. For example, for the shufflon adjacent to the pilV adhesin gene of the IncI2 family of conjugative plasmids, each shufflon's segment confers to the PilV adhesin binding affinities to specific receptors. Therefore, when a shufflon segment is aligned to an adhesin gene, it modulates the binding affinity of the corresponding adhesin protein. When a donor bacterium displays an adhesin, a shufflon can thus be used to influence the stability of the interaction between the donor bacterium and the target bacterium. Shufflons include, but are not limited to the following DNA sequences Shufflase recognition sites 5′-GTGCCAATCCGGTNNNTGG-3′ (SEQ ID NO: 140, abbreviated srs), alternative ORF to be re-arranged (altORFs). As such, one or more genes encoding one or more adhesin proteins present in the mating pair stabilization module can possess a shufflon. In an embodiment, the DNA sequence of the shufflon can be derived from one of the following family of bacterial conjugative plasmids IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc4, Inc7, Inc8, Inc9, Inc11, Inc13, Inc14, Inc18. In another embodiment, the DNA sequence of the shufflon can be derived from one of the incompatibility family of bacterial conjugative plasmids belonging to the I-complex: IncI1, IncI2, Incly, IncB/O (Inc10), IncK and/or IncZ. In another embodiment, the DNA sequence of the shufflon can be derived from one of the Inch family of bacterial conjugative plasmids. In yet another embodiment, the DNA sequence of the shufflon can be derived from one of the IncI2 family of bacterial conjugative plasmids. In yet another embodiment, the DNA sequence of the shufflon can be derived from the bacterial vector TP114.

When the gene encoding the adhesin comprises a shufflon, the mating pair stabilization module comprises one or more genes encoding a shufflase. Shufflases are recombinases capable of reorganizing the shufflon's DNA inversions segments which, as indicated above, can affect the binding activity and specificity of adhesin proteins. Shufflases include, but are not limited to one or more of rci (TP114-031). As such, the mating pair stabilization module can include the one or more genes encoding the one or more shufflase proteins. In addition, one or more shufflase, as well as, one or more different types of shufflase can be encoded by the mating pair stabilization module and expressed by the donor bacterium. In an embodiment, the genes encoding shufflases can be derived from one of the following family of bacterial conjugative plasmids IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inch, Inc4, Inc7, Inc8, Inc9, Inc11, Inc13, Inc14, Inc18. In another embodiment, the shufflon and/or shufflase can be derived from one of the incompatibility family of bacterial conjugative plasmids belonging to the I-complex: IncI1, IncI2, Incly, IncB/O (Inc10), IncK and/or IncZ. In another embodiment, the genes encoding the shufflases can be derived from one of the Inch family of bacterial conjugative plasmids. In yet another embodiment, the genes encoding the shufflases can be derived from one of the IncI2 family of bacterial conjugative plasmids. For example, the genes encoding shufflases can be derived from the bacterial vector TP114.

The mobilization module encodes a relaxosome, e.g., a protein complex capable of recognizing the transport module (an origin of transfer (oriT)) which is operatively associated with the payload module and subsequently transfers the genetic cargo through the conjugative pore into the recipient bacterium. The mobilization module (which can be heterologous to the conjugative bacterial cell) can be integrated in the chromosome of the conjugative bacterial cell or can be located on one or more bacterial vectors. The mobilization module includes, but are not limited to one or more of virC1 (TP114-68: parA), (TP114-41: nikB) and/or (TP114-42: nikA). The mobilization module can be derived from at least one of the following conjugative families MOB_{F}, MOB_{P}, MOB_{V}, MOB_{H}, MOB_{C} and/or MOB_{Q}. In another embodiment, the genes encoding the mobilization machinery can be derived from one of the MOB_{P} family of bacterial conjugative plasmids. For example, the genes encoding the mobilization machinery can be derived from the bacterial vector TP114. In yet another example, the genes encoding the mobilization machinery can be derived from the bacterial vector R6K. In another embodiment, the genes encoding the mobilization machinery can be derived from one of the MOB_{F} family of bacterial conjugative plasmids. For example, the genes encoding the mobilization machinery can be derived from the bacterial vector pOX38.

The transport module is a component of the genetic cargo which can also be present in the transfer machinery when the elements of the genetic cargo and of the transfer machinery are in cis organization. The transport module includes one or more functional DNA elements acting as an origin of transfer (ori7) of the genetic cargo into the recipient bacterium. The transport module may be heterologous to the conjugative bacterial cell. The transport module is cis-acting and is thus found on the genetic component (chromosome or extrachromosomal vector) comprising the genetic cargo. As indicated above, the transport module is acted upon by the mobilization module. As used in the context of the present disclosure, the term “mobilization” refers to the process by which a conjugative plasmid accomplishes the transfer, from a donor bacterium to a recipient bacterium, of a DNA molecule that contains an origin of transfer (oriT). The term “origin of transfer” (abbreviated ori7) refers to a DNA sequence that, when present in a DNA molecule, is recognized by the corresponding mobilization proteins and allows its mobilization.

The regulatory module, when present in the transport machinery, can include one or more genes and regulatory elements encoding one or more proteins or non-coding RNAs capable of regulating the expression of genes or capable of being used to regulate the expression of genes (e.g., an activator, a repressor, a riboswitch, CRISPR-Cas9, Zinc Finger Nuclease (ZFN), a TALE, taRNA). The regulatory module (which can be heterologous to the conjugative bacterial cell) can be integrated in the genome of the conjugative bacterial cell or located on one or more bacterial vector. In an embodiment, the regulatory genes and elements can be on a distinct nucleic acid molecule than the modules of the transfer machinery or of the conjugative delivery system. In another embodiment, the regulatory genes and elements can be isolated from different sources such as, but not limited to, the same plasmid as the other modules, another plasmid, a bacterial chromosome, a phage, a eukaryote chromosome, an archaebacterium. In yet another embodiment, the regulatory genes and elements can be engineered or evolved from naturally occurring genes. The regulatory proteins or non-coding RNAs encoded by the regulatory module can be used to induce or repress genes located on the chromosome of the bacterium hosting the delivery system, as well as to induce or repress genes located on any of the modules of the transfer machinery or of the genetic cargo. In an embodiment, the regulatory module includes one or more genes encoding a one or more regulatory proteins or non-coding RNAs such as, but not limited to, yajA (TP114-058), yafA (TP114-069), yaeC (TP114-070), yheC (TP114-085), fur, fnr, korA, acaCD, acr1, acr2, stbA, twrA, ResP, kfrA, ardK, Cas9, crRNA, ZFN, TALEN, taRNA, toehold switch, araC, tetR, lacI and/or lacIq.

When the transfer machinery is located (in total or in part) in an extrachromosomal vector, the extrachromosomal vector includes a vegetative replication module. The vegetative replication module of the transfer machinery can be the same or different from the vegative replication module of the genetic cargo. A vegetative replication module is required when the transfer machinery is located on one or more vector that replicate independently from the genome of the bacterial host. In that case, the one or more extrachromosomal vectors containing the transfer machinery need a vegetative replication module to replicate and be maintained in the bacterial host. The vegetative replication module comprises one or more functional DNA elements acting as an origin of vegetative replication (oriV). The oriV is a DNA sequence present on a genetic element that, when recognized by the replication machinery encoded by the maintenance module, allows a plasmid to replicate in a bacterial host. For versatile use, and for the maintenance of vectors in a large range of bacterial hosts, the oriV of the vegetative replication module can be a broad-host-range oriV (i.e. and oriV recognized by a broad range of bacterial host species). In some embodiments, where the maintenance of vectors needs to be restricted to a limited range of bacterial hosts, it may be preferable to use an oriV with a restricted or narrow host range (i.e. an oriV recognized by a limited range of bacterial host species).

When the transfer machinery is located (in totally or in part) on an extrachromosomal vector, the conjugative bacterial host cell includes a maintenance module (which can be considered, in some embodiments, part of the transfer machinery). The maintenance module includes proteins (referred to as replication machinery) capable of recognizing the oriV of the vegetative replication module and allows the replication of extrachromosomal vectors (e.g., plasmids) containing the vegetative replication module. The maintenance module includes proteins capable of recognizing the oriV of the vegetative replication module and allows the replication of plasmids containing the vegetative replication module. The maintenance module can be heterologous to the conjugative bacterial cell. When the maintenance of vectors needs to be restricted to the donor bacterium, it may be preferable to locate (e.g., integrate) the maintenance module into the donor bacterium chromosome. Alternatively, the maintenance module may be located on one or more extrachromosomal vector. The maintenance module can also comprise one or more genes and regulatory elements responsible for adequate DNA partitioning. The genes responsible for partitioning may include proteins responsible for equal segregation of plasmid copies into daughter cells, toxin and anti-toxin stabilization system and/or helicase and DNA primase which helps the replicative machinery in the replication of the plasmid. The proteins of the maintenance module include, but are not limited to one or more of proteins often annotated as repA (TP114-083: repA), TP114-082, parA (TP114-068: parA), parB, DNA primase (TP114-006: ygiA), a toxin (e.g. vcrx028 from pVCR94, TP114-051: ycfA from TP114), an antitoxin (e.g. vcrx027 from pVCR94, TP114-050: ycfB from TP114), DNA topoisomerases (TP114-035: ydiA and TP114-036: ydgA). As such, the maintenance module includes the one or more genes encoding the one or more proteins of the replicative machinery and can also include zero or more genes and regulatory elements coding for DNA partitioning protein. In addition, one or more replicative machinery, as well as, one or more different types of replicative machinery can be present in the maintenance module. In an embodiment, the maintenance module and/or the vegetative replication module can be derived from one of the following family of bacterial vectors IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 and/or Inc18. In an embodiment, maintenance module and/or the vegetative replication module can be derived from one of the IncI2 family of bacterial vectors. For example, maintenance module and/or the vegetative replication module can be derived from the bacterial vector TP114.

The transfer machinery can also include one or more selection module. The selection module includes one or more genes conferring a selectable trait for identifying bacteria bearing one or more modules of the transfer machinery. The selection module is operatively connected with the one or more bacterial vector and/or the integrated modules of the transfer machinery. The selection module of the transfer machinery can be the same or different from the selection module of the genetic cargo. The selectable trait can be, but is not limited to, an antibiotic resistance gene, a gene coding for a fluorescent protein (including a green fluorescent protein), an auxotrophic selection marker, a gene coding for a β-galactosidase (e.g., the bacterial IacZ gene), a gene coding for a luciferase, a gene coding for a chloramphenicol acetyltransferase (e.g., the bacterial cat gene), a gene coding for a β-glucuronidase.

The exclusion module, when present in the transfer machinery, includes one or more of genes encoding exclusion proteins. The exclusion module (which can be endogenous or heterologous to the conjugative bacterial cell) can be located in the bacterial chromosome or in one or more extrachromosomal vectors. Exclusion proteins limit the horizontal transfer of genetic material by rendering a bacterium resistant to conjugative plasmids. For example, a bacterium that expresses exclusion proteins (e.g., excAB) against a specific bacterial conjugative plasmid (e.g., R64) can no longer receive this plasmid through conjugation. This phenomenon can be used to avoid futile conjugative transfer between conjugative bacterial cell bacteria. For instance, if conjugative bacterial cell bacteria are designed to express an exclusion protein directed against their own transfer machinery used to propagate the genetic cargo, transfer between conjugative bacterial cell bacteria can no longer occur (or at significantly lower rates). Exclusion proteins include, but are not limited to one or more of TP114-05 from TP114, excA and excB from plasmid R64, trbK from RP4, traS and traT from plasmid F (pOX38). As such, the exclusion module can include one or more genes encoding one or more exclusion proteins. In an embodiment, the genes encoding exclusion proteins can be derived from one of the following family of bacterial conjugative plasmids IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14, Inc18. In another embodiment, the genes encoding exclusion proteins can be derived from one of the IncI2 family of bacterial conjugative plasmids. For example, the genes encoding exclusion proteins can be derived from the bacterial vector TP114.

Genetic Cargo

The genetic cargo is intended to be delivered by the conjugative bacterial cell donor bacterium to a target bacterium via the transfer machinery. The genetic cargo includes genes and regulatory elements which are divided in different modules further described below. The genes present within those modules can optionally be organized in the form of one or more operons.

The genetic cargo comprises a payload module which is operatively associated with a transport module. The transport module is “operatively associated” with the payload module which allows the transfer of the payload module when the proteins encoded by the mobilization module associate with the transport module. The genetic cargo is heterologous to the conjugative bacterial host cell because at least one of the payload module or the transport module has been genetically introduced in the conjugative bacterial host cell in order to operatively associate the transport module with the payload module. The genetic cargo can optionally include a selection module, a vegetative replication module and/or a mobilization module.

The payload module can include, but is not limited to, genes, regulatory elements, non-coding RNAs (such as siRNAs, shRNAs and miRNAs for example), transposons, genomes (e.g., phage, or bacterial). In a specific embodiment, the payload module encodes a guide RNA (gRNA) and/or a CRISPR-array (crRNA and tracrRNA) that can be recognized and acted upon by the recipient cell. The payload module can encode for one or more proteins, and/or one or more non-coding genetic elements (such as RNA for example). The payload module can also be a combination of one or more genes, and/or regulatory elements, and/or non-coding RNA, and/or transposons, and/or genome.

In a specific embodiment, the payload module includes one or more heterologous genes encoding one or more heterologous proteins or functional RNA which are intended to be expressed in a recipient bacterium. In the context of the present disclosure, the expression of the heterologous gene(s) in the recipient bacterium can be beneficial, neutral or detrimental to the recipient bacterium. An heterologous gene is considered beneficially expressed in a recipient bacterium when its expression causes a biological advantage to the recipient bacterium. Beneficially expressed heterologous genes include, but are not limited to lacZ, lacy, lacA, galE, galT, galK, gadD, gadT, gadP, scrA, scrB, merA, AN-PEP. An heterologous gene is considered neutrally expressed in a recipient bacterium when its expression does not provide a biological advantage and also fails to provide a biological disadvantage to the recipient bacterium. Neutrally expressed heterologous genes include, but are not limited to, proteins exhibiting a therapeutic benefit to the subject having the recipient bacterium (e.g., therapeutic proteins such as eukaryotic growth factors, hormones (e.g., glucagon-like peptide-1 or GLP-1, insulin, etc.), cytokines including interleukins (e.g., interleukin 1, 2, 3, 4, 5, 6, 7, 8, 9, 10, 11, 12, 13, 14, 15, 16 or 17), and/or chemokines (e.g., CC chemokines, CXC chemokines, C chemokines or CX3C chemokines). An heterologous gene is considered detrimentally expressed in a recipient bacterium when its expression provides a biological disadvantage to the recipient bacterium (for example, a reduction in cell growth, an increase in sensitivity to an antibiotic and/or an increase in mortality). Detrimentally expressed heterologous gene include, but are not limited to, nucleases (for example, transcription activator-like effector nucleases (TALEN), zinc finger nucleases (ZFN) and clustered regularly interspaced short palindromic repeat (CRISPR) associated DNA-binding (Cas) proteins and analogs thereof, endonuclease restriction enzymes (e.g., ApaLI, BamHI, BgIII, DpnI, EcoR1, EcoRV, HindIII, PvuI, PvuII, XhoI), and toxins or protein toxic for the recipient bacterium (e.g. Lysins, Vcrx028, MazF, HicB, KikA, CcdB, microcins).

In a specific embodiment, the heterologous protein encoded by the payload module is a Cas protein or a Cas protein analog. As used in the context of the present disclosure, a Cas protein or an associated analog is an endonuclease capable of mediating a double-strand cut (either blunt or staggered) in a DNA molecule at the specific location where a CRISPR RNA (crRNA) localizes on the DNA molecule. The Cas protein can be a type I, type II, or type III CRISPR RNA-guided endonuclease. In the context of the present disclosure, a “Cas protein analog” refers to a variant of the Cas protein, or to a fragment of the Cas protein, capable of mediating a double-strand cut (either blunt or staggered) in a DNA molecule at the specific location where a CRISPR RNA (crRNA) localizes on the DNA molecule, or capable of mediating a single stand cut in a DNA or RNA molecule at the specific location where a CRISPR RNA (crRNA) localizes on the DNA or RNA molecule.

A Cas protein variant comprises at least one amino acid difference when compared to the amino acid sequence of the native Cas protein. As used herein, a variant refers to alterations in the amino acid sequence that does not adversely affect the biological functions of the Cas protein analog. A substitution, insertion or deletion is said to adversely affect the protein when the altered sequence prevents or disrupts a biological function associated with the Cas protein. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the Cas protein. The Cas protein variants have at least 50%, 55%, 60%, 65%, 70%, 75%, 80%, 85%, 90%, 95%, 96%, 97%, 98% or 99% identity to the Cas proteins described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant Cas proteins described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature protein is fused with another compound, such as a compound to increase the half-life of the protein, or (iv) one in which the additional amino acids are fused to the mature protein for purification of the polypeptide. A “variant” of the Cas protein can be a conservative variant or an allelic variant.

The Cas protein analog can be a fragment of a known/native Cas proteins. Cas protein “fragments” (including baking enzyme “fragments”) have at least at least 100, 200, 300, 400, 500, 600, 700, 800, 900 or more consecutive amino acids of the Cas protein. A fragment comprises at least one less amino acid residue when compared to the amino acid sequence of the known/native Cas protein and still possess the endonucleic activity of the full-length Cas protein. In some embodiments, fragments of the Cas proteins can be employed for producing the corresponding full-length Cas proteins by peptide synthesis. Therefore, the fragments can be employed as intermediates for producing the full-length proteins. In some embodiments, the Cas protein fragments can have at least 50%, 55%, 60%, 65%, 70%, 75%, 80%, 85%, 90%, 95%, 96%, 97%, 98% or 99% identity to the Cas proteins described herein.

In an embodiment, the Cas protein is a Cas9 protein and allows for the formation of blunt ends at the cleavage site. In an embodiment, the Cas9 protein can be derived, for example, from Streptococcus pyogenes. The Cas9 protein acts in collaboration with a CRISPR RNA (crRNA) moiety and trans-activating CRISPR RNA (tracrRNA) moiety to specifically cleave double-stranded DNA. The crRNA moiety can be specific to a nucleic acid sequence in a double stranded DNA (present in the recipient bacterium for example), and in the presence of such nucleic acid sequence and the Cas9 protein, forms a duplex with the nucleic acid sequence to specifically direct the Cas9 endonuclease activity in the duplex region. The tracrRNA specifically binds to the Cas9 protein and allows a close association with the crRNA. In an embodiment in which the Cas9 protein is the heterologous protein, the payload module can also include a gene encoding the crRNA and/or the tracrRNA. In another embodiment in which the Cas9 protein is the heterologous protein, the payload module nucleic acid molecule can comprise a gene coding for a guide RNA (gRNA). The gRNA includes, on the same gene transcript, both a CRISPR RNA (crRNA) and trans-activating CRISPR RNA (tracrRNA).

In another embodiment, the Cas protein is a Cpf1 protein and allows for the formation of staggered ends at the cleavage suite. In an embodiment, the Cpf1 protein can be derived, for example, from Francissella novicida. Unlike the Cas9 protein, the Cpf1 protein only requires the presence of crRNA to mediate specific cleavage of the double stranded DNA. As such, in embodiments in which the Cpf1 protein is used as the Cas protein, the payload module includes a CRISPR RNA (crRNA) and does not need to include a trans-activating CRISPR RNA (tracrRNA).

The present disclosure provides that the crRNA found on the payload module is recognizable by the Cas protein. This means that the crRNA is able to direct the endonuclease of a type I or type II Cas protein to a specific location on a double stranded DNA molecule, or to direct the endonuclease of a type III Cas protein to a specific location on a RNA molecule. Since that, in such embodiments, what is required is that the crRNA forms a duplex at one or more specific location (e.g., one or more target location) in the recipient bacterium genome, or at one or more specific location on RNA molecules of the recipient bacterium, then the crRNA must be substantially complementary to the one or more target location on the genome in the recipient bacterium, or on RNA molecules present in the recipient bacterium. As used herein, the term “genome” includes the chromosomal and plasmidic DNA of a bacterium. As also used herein, the term “substantially complementary” refers to the sequence of the crRNA having a minimal level of complementary so as to allow it to form a specific duplex with the one or more target location in the recipient bacterium genome, or RNA molecules present in the recipient bacterium.

In one embodiment, the crRNA is substantially complementary to a target sequence present in single or multiple copies in the recipient bacterium. In such embodiment, the transfer of the genetic cargo in the recipient bacterium will allow for the expression of the crRNA (which will form a plurality of duplexes in the recipient bacterium) and the Cas protein in the recipient bacterium which will eventually lead to the formation of multiple double-strand DNA cuts in the target bacterial genome. These multiple double-strand DNA cuts will eventually lead to a reduction in the viability of the recipient bacterium, most likely, in the death of the recipient bacterium.

In embodiments in which killing of the recipient bacterium is not desired (for example to avoid inflammatory reactions triggered by the death of a population of recipient bacterium), the crRNA can be substantially complementary to a single location in the genome of the recipient bacterium, for example, a specific gene in a recipient bacterium. The payload module would also have to contain a DNA molecule that can be used as a template to repair the target locus and introduce an inactivating mutation that also can protect from the crRNA targeting. For example, the crRNA can be substantially complementary to a gene coding for a virulence factor in the recipient bacterium, or an RNA coding for a virulence factor in the recipient bacterium. In such embodiments, the introduction of the payload module will lead to the inactivation of the virulence factor by introducing the mutated reparation template into the virulence factor gene without altering the viability of the recipient bacterium or causing deleterious effects in the subject bearing the recipient bacterium. The virulence factor can be located on the chromosome of the recipient bacterium or on a plasmid of the recipient bacterium.

The virulence factor in the recipient bacterium can be for example a gene conferring resistance to a drug, such as, for example, an antibiotic. The term “antibiotic resistance gene” encompasses a gene, or the encoding portion thereof, which encodes a protein or transcribes a functional RNA that confers antibiotic resistance. For example, the antibiotic resistance gene may be a gene or the encoding portion thereof which contributes to (1) an enzyme which degrades an antibiotic, (2) an enzyme which modifies an antibiotic, (3) a pump such as an efflux pump for the antibiotic, or (4) a mutated target which suppresses the effect of the antibiotic. Gene coding for an antibiotic resistance trait include, but are not limited to, the aadA2, aadA, aacC, aacA1, aphA, strAB, pbp1A, pbp1B, pbp2A, pbp2B, dac, bla_{CMY-2}, floR, cmlA, cat, cmx, ermA, mph2, mel, erm(x), mecA, aadA1a, sul1, sul2, tetA, tet(W), blaSHV-1, dhfr, van(A), van(B) and bla_{NDM}1.

The virulence factor in the recipient bacterium can be, for example, a gene encoding a toxin. Gene coding for a toxin include, but are not limited to, ccdB, relE, parE, doc, vapC, hipA, stl, espA, pag, ctxA, ctxB, tcpA, exoU, exoS, exoT, SgiT and hipB.

The virulence factor can be a structure or a component, such as a pilus, a fimbriae, a flagella or pumps. Gene encoding for virulent component include, but are not limited to fimA, csgD, toxT, cps, ptk, epsA, mia, ssrB, acrA, acrB, tolC and csgA.

In a specific embodiment, the crRNA is specific to genes, or to RNA molecules derived from genes, coding for a virulence factor found in an Escherichia sp., such as, for example, a gene coding for a virulence factor in Escherichia coli. Virulence factors found in Escherichia coli include, but are not limited to, those described in WO2015/148680. In a specific embodiment, genes encoding a virulence factor include antibiotic resistance genes and shiga toxin genes in Escherichia coli (e.g., multidrug resistance shiga-toxin producing E. coli). In another specific embodiment, the genes encoding the virulence factor include gene coding for a pilus (e.g., for example a type 1 pilus) in Escherichia coli (e.g., adherent-invasive E. coli).

The transport module is a component of the genetic cargo and includes a functional DNA locus responsible for the physical transport of the genetic cargo into the recipient bacterium. The transport module comprises an origin of transfer (oriT), e.g., a nucleic acid sequence allowing the transfer of a vector from the donor bacterium to the recipient bacterium. The transport module may be heterologous to the conjugative bacterial cell. The transport module is cis-acting and is thus found on the genetic component (chromosome or extrachromosomal vector) comprising the genetic cargo. As indicated above, the transport module is acted upon by the mobilization module. As used in the context of the present disclosure, the term “mobilization” refers to the process by which a conjugative plasmid accomplishes the transfer, from a donor bacterium to a recipient bacterium, of a DNA molecule that contains an origin of transfer (oriT). The term “origin of transfer” (abbreviated oriT) refers to a DNA sequence that, when present in a DNA molecule, is recognized by the corresponding mobilization proteins and allows its mobilization.

The genetic cargo can also include one or more selection module. The selection module includes one or more genes conferring a selectable trait for identifying bacteria bearing one or more modules of the genetic cargo. The selection module is operatively connected with the one or more bacterial vector and/or the integrated modules of the genetic cargo. The selection module of the genetic cargo can be the same or different from the selection module of the conjugative delivery system. The selectable trait can be, but is not limited to, an antibiotic resistance gene, a gene coding for a fluorescent protein (including a green fluorescent protein), an auxotrophic selection marker, a gene coding for a β-galactosidase (e.g., the bacterial lacZ gene), a gene coding for a luciferase, a gene coding for a chloramphenicol acetyltransferase (e.g., the bacterial cat gene), a gene coding for a β-glucuronidase.

When the genetic cargo is located (in total or in part) in an extrachromosomal vector, the extrachromosomal vector includes a vegetative replication module. The vegetative replication module of the genetic cargo can be the same or different from the vegative replication module of the conjugative delivery system. A vegetative replication module is required when the transfer machinery is located on one or more vector that replicate independently from the genome of the bacterial host. In that case, the one or more extrachromosomal vectors containing the genetic cargo need a vegetative replication module to replicate and be maintained in the bacterial host. The vegetative replication module comprises one or more functional DNA elements acting as an origin of vegetative replication (oriV). The oriV is a DNA sequence present on a genetic element that, when recognized by the replication machinery encoded by the maintenance module, allows a plasmid to replicate in a bacterial host. For versatile use, and for the maintenance of vectors in a large range of bacterial hosts, the oriV of the vegetative replication module can be a broad-host-range oriV (i.e. and oriV recognized by a broad range of bacterial host species). In some embodiments, where the maintenance of vectors needs to be restricted to a limited range of bacterial hosts, it may be preferable to use an oriV with a restricted or narrow host range (i.e. an oriV recognized by a limited range of bacterial host species).

When the genetic cargo is located (in totally or in part) on an extrachromosomal vector, the conjugative bacterial host cell includes a maintenance module (which can be considered, in some embodiments, part of the transfer machinery). The maintenance module includes proteins (referred to as the replication machinery) capable of recognizing the oriV of the vegetative replication module and allows the replication of extrachromosomal vectors (e.g., plasmids) containing the vegetative replication module. The maintenance module includes proteins capable of recognizing the oriV of the vegetative replication module and allows the replication of plasmids containing the vegetative replication module. The maintenance module can be heterologous to the conjugative bacterial cell. When the maintenance of vectors needs to be restricted to the donor bacterium, it may be preferable to locate (e.g., integrate) the maintenance module into the donor bacterium chromosome. Alternatively, the maintenance module may be located on one or more of an extrachromosomal vector. The maintenance module can also comprise one or more genes and regulatory elements responsible for adequate DNA partitioning. The genes responsible for partitioning may include proteins responsible for equal segregation of plasmid copies into daughter cells, toxin and anti-toxin stabilization system and/or helicase and DNA primase which helps the replicative machinery in the replication of the plasmid. The proteins of the maintenance module include, but are not limited to one or more proteins often annotated as repA (TP114-083: repA), TP114-082, parA (TP114-068: parA), parB, DNA primase (TP114-006: ygiA), a toxin (e.g. vcrx028 from pVCR94, TP114-051: ycfA from TP114), an antitoxin (e.g. vcrx027 from pVCR94, TP114-050: ycfB from TP114), DNA topoisomerases (TP114-035: ydiA and TP114-036: ydgA). As such, the maintenance module includes the one or more genes encoding the one or more proteins of the replicative machinery and can also include zero or more genes and regulatory elements coding for DNA partitioning protein. In addition, one or more oriV and replicative machinery, as well as, one or more different types of oriV and replicative machinery can be present in the maintenance module. In an embodiment, the maintenance module and/or the vegetative replication module can be derived from one of the following family of bacterial vectors IncA, IncB/O (Inc10), IncC, IncD, IncE, IncF1, IncF2, IncG, IncHI1, IncHI2, IncI1, IncI2, IncJ, IncK, IncL/M, IncN, IncP, IncQ1, IncQ2, IncR, IncS, IncT, IncU, IncV, IncW, IncX1, IncX2, IncY, IncZ, ColE1, ColE2, ColE3, p15A, pSC101, IncP-2, IncP-5, IncP-7, IncP-8, IncP-9, Inc1, Inc4, Inc7, Inc8, Inc9, Inc11, Inc13, Inc14 and/or Inc18. In an embodiment, maintenance module and/or the vegetative replication module can be derived from one of the IncI2 family of bacterial vectors. For example, maintenance module and/or the vegetative replication module can be derived from the bacterial vector TP114.

The mobilization module encodes a relaxosome, e.g., a protein complex capable of recognizing the transport module (an origin of transfer (oriT)) which is operatively associated with the genetic cargo and subsequently transferring the genetic cargo through the conjugative pore into the recipient bacterium. The mobilization module (which can be heterologous to the conjugative bacterial cell) can be integrated in the chromosome of the conjugative bacterial cell or can be located on one or more bacterial vectors. The mobilization module includes, but are not limited to one or more of virC1 (TP114-68: parA), (TP114-41: nikB) and/or (TP114-42: nikA). The mobilization module can be derived from at least one of the following conjugative families MOB_{F}, MOB_{P}, MOB_{V}, MOB_{H}, MOB_{C} and/or MOB_{Q}. In another embodiment, the genes encoding the mobilization machinery can be derived from one of the MOB_{P} family of bacterial conjugative plasmids. For example, the genes encoding the mobilization machinery can be derived from the bacterial vector TP114. In yet another example, the genes encoding the mobilization machinery can be derived from the bacterial vector R6K. In another embodiment, the genes encoding the mobilization machinery can be derived from one of the MOB_{F} family of bacterial conjugative plasmids. For example, the genes encoding the mobilization machinery can be derived from the bacterial vector pOX38.

Configurations of the Conjugative Delivery System

In a specific embodiment, the conjugative delivery system is designed to provide cis mobilization to allow exponential dissemination of the genetic cargo. In such embodiment, all of the modules of the system are located on a single extrachromosomal vector (in some embodiments, a circular plasmid). Using a system based on cis mobilization provides very limited to no containment, allowing the transfer of the conjugative plasmid to the recipient cell and subsequent rounds of transfers from the recipient cell to other recipient cells as well as the replication of the conjugative plasmid in the recipient cells.

In another specific embodiment, the conjugative delivery system is designed to provide a constrained cis mobilization to allow rapid dissemination of the genetic cargo and provide a certain degree of containment. In such embodiment, the maintenance module is located in the conjugative bacterial cell's chromosomes and the remaining modules of the system are located on a single extrachromosomal vector (in some embodiments, a circular plasmid). Using a system designed to provide constrained cis mobilization offers some level of containment, allowing transfer of the conjugative plasmid to the recipient cell, and subsequent transfers from the recipient cell to other recipient cells, but preventing its replication in the recipient cells.

In a further specific embodiment, the conjugative delivery system is designed to provide in trans mobilization to increase the level of containment of the genetic cargo. In such embodiment, the entire transfer machinery is located in the conjugative bacterial cell's chromosomes or is located on one or many extrachromosomal vector (in some embodiments, a circular plasmid) but lacks the transport module. The modules of the genetic cargo (payload module and transport module) of the system are located on a single extrachromosomal vector (in some embodiments, a circular plasmid). In such embodiment, the genetic cargo would also include a vegetative replication module. Using a system designed to provide in trans mobilization offers the highest level of containment, preventing the transfer of the mobilized plasmid from a recipient cell to another recipient cell, and preventing replication in the recipient cells.

The modules of the genetic cargo can also be integrated in the bacterial chromosome. In such embodiment, the genetic cargo could either be excised or include a vegetative replication module upstream (in operative association) with the payload module.

The system of the present disclosure is designed to allow the transfer of a genetic cargo to a recipient bacterium in order to express one or more heterologous proteins and/or one or more non-coding DNA or RNA molecules, in the recipient bacterium. The system, when introduced in a donor bacterium, allows the genetic cargo to be transferred to target bacteria at an acceptable conjugation efficiency of in vivo (e.g. in the gastro-intestinal environment). As used in the context of the present disclosure, “conjugation efficiency” refers to a measure of the transfer of the genetic cargo from the donor bacterium to the recipient bacterium. A conjugation efficiency can be determined in vivo (e.g., in a subject) or in vitro (e.g., outside a subject, in a (liquid or solid) culture medium, for example) in numerous ways by the person skilled in the art. In embodiments in which the conjugation efficiency is measured in vivo, it can be provided as the number of bacterial exconjuguants (e.g., the number of bacteria that have received the genetic cargo from the conjugative bacterial cell bacterium) per total available recipient bacterium. Conjugation efficiency can be measured in a specific location in the subject, for example in the gut of the subject (and in such instance, a level of enteric conjugation efficiency is provided). As used in the context of the present disclosure, the expression “an acceptable level of in vivo conjugation efficiency” refers to a level of conjugation, observed in vivo, capable of providing sufficient transfer of the genetic cargo to mediate significant impact on, or by, the target cell population. In some embodiments, the system has an in vivo conjugation efficiency of at least 10⁻³, 10⁻² or 10⁻¹ transconjugant bacterium/recipient bacterium. In a specific embodiment, the system has an in vivo conjugation efficiency of at least 10⁻³ transconjugants bacterium/recipient bacterium. In a specific embodiment, the system has an in vivo conjugation efficiency of at least 10⁻² transconjugant bacterium/recipient bacterium. In a specific embodiment, the system has an in vivo conjugation efficiency of at least 10⁻¹ transconjugant bacterium/recipient bacterium.

As shown in the present disclosure, in some embodiments, conjugation efficiencies are compared in several mating conditions. As such, a measure of in vitro conjugation under certain conditions can be used as a proxy for determining if the in vivo transfer efficiency of a vector is acceptable. Consequently, in some embodiments, conjugation efficiency of the system under hypoxic conditions, presence of feces in the medium, physiologically relevant temperature (e.g., 37° C.), unstable mating environment (e.g. a static or agitating broth) is at least 10⁻³, 10⁻² or 10⁻¹ transconjugant bacterium/recipient bacterium as compared with standard solid medium mating. In a specific embodiment, the system has an in vitro conjugation efficiency, when measured in any of the above conditions, of at least 10⁻³ transconjugant bacterium/recipient bacterium. In a specific embodiment, the system has an in vitro conjugation efficiency, when measured in any of the above conditions, of at least 10⁻² transconjugant bacterium/recipient bacterium. In a specific embodiment, the system has an in vitro conjugation efficiency, when measured in any of the above conditions, of at least 10⁻¹ transconjugant bacterium/recipient bacterium.

In some embodiments, a ratio between the conjugative efficiency in a liquid medium vs. a solid medium can be used as a as a proxy for determining if the in vivo transfer efficiency of a vector is acceptable. As shown in the Examples below, a ratio of conjugative efficiency higher than 0.1, 0.2, 0.3, 0.4, 0.5, 0.6, 0.7, 0.8, 0.9 or 1.0% is indicative that the conjugative bacterium has acceptable in vivo transfer efficiency. In some embodiments, a ratio of conjugative efficiency higher than 0.1% is indicative that the conjugative bacterium has acceptable in vivo transfer efficiency.

The present disclosure also includes a method for determining the efficiency of in vivo transfer by measuring the ability of a bacterial system to conjugate in a liquid medium. Such method includes contacting a conjugative bacterial host cell and a recipient bacterial host cell in a liquid medium and determining the conjugation efficacy in such liquid medium. In an embodiment, the liquid medium has a viscosity substantially similar to water, when measured at a specific temperature (37° C. for example). If the conjugation efficacy is at least 10⁻³, 10⁻² or 10⁻¹ transconjugant bacterium/recipient bacterium as compared with standard solid medium mating, then it is determined that the conjugative bacterial cell will successfully be able to conjugate in vivo (in the gastro-intestinal tract of a subject for example). Alternatively or in combination, the method can include determining a ratio between the conjugative efficiency in a liquid medium vs. a solid medium. In such embodiment, a ratio of conjudative efficiency higher than 0.1, 0.2, 0.3, 0.4, 0.5, 0.6, 0.7, 0.8, 0.9 or 1.0% is indicative that the conjugative bacterium has acceptable in vivo transfer efficiency. The contact between the conjugative bacterial cell and the recipient bacterial cell can be done at a specific temperature which is the same or substantially similar to the in vivo environment, e.g., between 30 and 40° C. (37° C. for example). The contact between the conjugative bacterial cell and the recipient bacterial cell can be done in static conditions or in the presence of an agitation.

Probiotic Recombinant Donor Bacteria, Compositions Comprising Same and Processes for Making Same

The present disclosure also provides a recombinant bacterial host cell (referred to as a conjugative bacterial cell) that can act as a donor bacterium capable of conjugation to transfer the genetic cargo described herein into a target (recipient) bacterium. The conjugative bacterial cell bacterium comprises the transfer machinery and the genetic cargo described herein. In some embodiments, the transfer machinery and the genetic cargo can be independently replicating from the genome of the recombinant bacterium. In such embodiment, the transfer machinery can be operatively associated with the genetic cargo nucleic acid molecule and form, for example, a single unitary vector (e.g., a single plasmid). In another embodiment, the transfer machinery can be integrated in the chromosome of the conjugative bacterial cell bacterium (at a single location or at multiple locations) and the genetic cargo nucleic acid molecule can be independently replicating from the genome of the donor bacterium. In another embodiment the donor bacterium can comprise at least two distinct vectors (e.g., two distinct plasmids): a first one comprising the transfer machinery and a second one comprising the genetic cargo nucleic acid molecule.

Since the transfer machinery of the present disclosure has a high in vivo conjugation efficiency, the amount of conjugative bacterial cell bacteria necessary to achieve a desired therapeutic effect in the subject is going to be equal or lower than other recombinant bacteria lacking the system of the present disclosure.

In some embodiments, the conjugative bacterial cell can be a pathogenic bacterial cell that has been modified to reduce or eliminate its pathogenicity. Alternatively, the conjugative bacterial cell of the present disclosure is considered to be a probiotic bacterium since these are, at the very least, not harmful (e.g., not pathogenic) to the subject, and in some embodiments, probiotics can by themselves confer a health benefit to the subject. The present disclosure thus provides a bacterium which has been genetically engineered to bear the delivery system of the present disclosure. Thus, the present disclosure also provides a process for obtaining the conjugative bacterial cell by introducing the system of the present disclosure in a bacterial cell. Optionally, the system can include a gene conferring one or more selectable traits.

Bacterial cells that can be used as conjugative bacterial cells include, but are not limited to, Bacillus sp., Bifidobacterium sp., Enterococcus sp., Escherichia sp., Lactobacillus sp., Lactococcus sp., Leuconostoc sp., Pediococcus sp. and Streptococcus sp. As such, the present disclosure provides a probiotic recombinant bacterium from the genus Bacillus sp., Bifidobacterium sp., Enterococcus sp., Escherichia sp., Lactobacillus sp., Lactococcus sp., Leuconostoc sp., Pediococcus sp. and Streptococcus sp. as well as a process for making such probiotic bacteria by introducing the conjugative bacterial vector or system in a probiotic bacterium of the genus Bacillus sp., Bifidobacterium sp., Enterococcus sp., Escherichia sp., Lactobacillus sp., Lactococcus sp., Leuconostoc sp., Pediococcus sp. and Streptococcus sp. Bacterial species which as considered probiotic to human subjects include, but are not limited to Bacillus coagulans (e.g., strain GBI-30 or 6086), Bifidobacterium animalis subsp. lactis (e.g., strain BB-12), Bifidobacterium longum subsp. infantis, Enterococcus durans (e.g. strain LAB18s), Escherichia coli (e.g., strain Nissle 1917), Lactobacillus acidophilus (e e.g., strain NCFM), Lactobacillus bifidus, Lactobacillus johnsonii (e.g., strain Lai, LCI or NCC533), Lactobacillus paracasei (e.g., strain Stl 1 or NCC2461), Lactobacillus plantarum (e.g., strain 299v), Lactobacillus reuteri (e.g., strain ATCC 55730, SD2112, Protectis, DSM 17938, Prodentis, DSM 17938, ATCC 55730, ATCC PTA 5289, RC-14), Lactobacillus rhamnosus (e.g., strain GG, GR-1) and Lactococcus thermophiles, Leuconostoc masenteroides (e.g. strain B7), Pediococcus acidilactici (e.g. strain UL5), Streptococcus thermophilus. As such, the present disclosure provides, in some embodiments, a conjugative bacterial cell recombinant bacterium from the bacterial species which are considered probiotic as well as a process for making such probiotic bacteria by introducing the conjugative bacterial vector or system in a probiotic bacterium of the strains of bacteria considered probiotic in humans. In a specific embodiment, the probiotic is from the genus Escherichia, for example the species Escherichia coli, e.g. E. coli Nissle. The present disclosure provides a process for making such probiotic recombinant bacteria by introducing the conjugative bacterial vector or system in a probiotic species Escherichia coli, e.g. E. coli Nissle.

In an embodiment, the recombinant donor bacterium is an enteric recombinant bacterium because it is capable of colonizing the gastro-intestinal tract of the subject receiving the recombinant bacteria. In an embodiment, the enteric recombinant bacterium is capable of colonizing the stomach, the intestine (including the small and the large intestine) and/or the colon of the subject receiving the recombinant bacteria

In an embodiment, the recombinant bacterium can be formulated as a composition (which can be a probiotic composition). The composition can also comprise an excipient, one or more antibiotic(s), a selection pressure (for selecting the cells having the selectable trait) and/or one or more chemically active molecules, and/or one or more strains of probiotic (non-recombinant) bacterium. In the composition, the recombinant bacterium can be provided as a solution/suspension or in a dried form. The composition can be provided for administration by any routes and, in an embodiment, the composition can be provided for oral administration, for injection, for inhalation, etc. When the composition is intended for oral administration and is used with the intention of colonizing the gastro-intestinal tract of a subject, care should be taken to formulate the recombinant bacterium to preserve its viability and its ability to perform conjugation until it reaches the desired location (suspected of comprising the recipient bacterium).

The present disclosure thus provides a process for making the composition. Broadly, the process comprises combining the recombinant bacterium with an excipient and optionally additional probiotic bacteria and/or antibiotics and/or chemically active molecules. The process can comprise making a solution/suspension of the recombinant bacterium or drying the recombinant bacterium. When the composition is intended for oral administration, the process for making the composition and the excipient used in the composition are designed/selected for allowing oral administration.

Therapeutic Uses of Recombinant Bacterial Host Cells and Compositions Comprising Same

The recombinant conjugative bacterial host cell of the present disclosure acts as a donor bacterium to transfer the genetic cargo to a target (recipient) bacterium. The transfer can occur in a subject (human or animal) to which a conjugative bacterial cell is to be administered. The subject can be suspected or is known to bear the recipient bacterium. The subject can be a human subject or an animal subject (such as, for example, a non-human mammal). In an embodiment in which the recombinant bacterium is an enteric bacterium, the transfer is intended to occur in the gastro-intestinal tract of the subject.

In an embodiment, the recombinant bacterium is selected or engineered to have a modification module which is the same or similar to the restriction-modification system of the intended recipient bacterium. In bacteria, there are four known restriction-modification systems (type I, II, III and IV) involved in the bacterial defence system against foreign DNA. Similarity in modification module will facilitate the introduction of the genetic cargo molecule in the recipient bacterium by protecting the DNA from restriction, thus increasing conjugation efficiency. For example, in embodiments in which the recipient bacterium has a type I restriction-modification system, a recombinant bacterium having a similar type I modification system, for example a recombinant bacterium from the species Escherichia coli, can be selected and used. In an embodiment, the restriction modification system is endogenous to the donor bacterium and is part of the exclusion module. In another embodiment, the restriction modification system is heterologous to the donor bacterium and is incorporated in an exclusion module. In an embodiment, the restriction modification system of the donor bacterium and the restriction modification system of the recipient bacterium comprises/is a type I restriction modification system. In another embodiment, the restriction modification system of the donor bacterium and the restriction modification system of the recipient bacterium comprises/is a type II restriction modification system. In a further embodiment, the restriction modification system of the donor bacterium and the restriction modification system of the recipient bacterium comprises/is a type III restriction modification system. In yet another embodiment, the restriction modification system of the donor bacterium and the restriction modification system of the recipient bacterium comprises/is a type IV restriction modification system.

The present disclosure thus provides a method of transferring a genetic cargo from a donor bacterium to a recipient bacterium in the microbiota of a subject in need thereof. The transfer can be done in a liquid (urine or blood for example) or in a solid surface (an epithelium for example). The microbiota may be located on a solid surface (such as the gastro-intestinal epithelium, the bladder epithelium or the lung epithelium) or in a liquid (such as in the urine of the bladder or the urethra, the blood in a blood vessel, the gastric juices or the stomach or the lymph in a lymph node for example). The method comprises administering a therapeutically effective amount of the conjugative bacterial cell bacterium of the present disclosure to the subject in need thereof. As used in the context of the present disclosure, a therapeutically effective amount refers to an amount (dose) effective in mediating a therapeutic benefit to the subject. It is also to be understood herein that a “pharmaceutically effective amount” may be interpreted as an amount giving a desired therapeutic effect, either taken in one dose or in any dosage or route, taken alone or in combination with other therapeutic agents. The method can also comprise determining the presence of the recipient bacterium in the subject prior to the administration of the recombinant bacterium. The method can further comprise determining if the restriction-modification system of the recombinant bacterium is substantially similar to the restriction-modification system of the intended recipient bacterium.

Advantageously, as indicated above, since the system of the present disclosure has high in vivo conjugation efficiency, the amount of recombinant bacteria necessary to achieve a desired therapeutic effect in the subject receiving the recombinant bacterium is going to be lower than other recombinant bacterium lacking conjugative delivery system of the present disclosure.

In the embodiments in which the genetic cargo encodes for one or more heterologous protein, and/or a non-coding RNA, and/or a phage genome, and/or a bacterial genome, the recipient bacterium can be any type of bacterium present in the subject which would accept conjugation from the recombinant bacterium. In such instances, the recipient bacterium can be, for example, part of the enteric microbiota (which can be or not pathogenic to the subject) which include, but is not limited to Aeromonas sp., Bacillus sp., Bifidobacterium sp., Campylobacter sp., Citrobacter sp., Clostridium sp., Enterobacter sp., Escherichia sp., Klebsiella sp., Hafnia sp., Helicobacter sp., Lactobacillus sp., Lactococcus sp., Morganella sp., Plesiomonas sp., Proteus sp., Providencia sp., Pseudomonas sp., Salmonella sp., Serratia sp., Shigella sp., Staphylococcus sp., Vibrio sp. and Yersinia sp.

In an embodiment in which the genetic cargo encodes one or more heterologous proteins, the bacterium receiving the genetic cargo can subsequently express one or more heterologous proteins. For example, the recipient bacterium can express one or more of a eukaryotic growth factor, and/or hormone, and/or cytokine (including an interleukin and/or a chemokine). The expression of the heterologous protein is intended to provide a therapeutic benefit to the subject having received the recombinant bacterium. For example, when the therapeutic protein is a hormone, like a GLP-1 peptide, the present disclosure provides using the recombinant bacterium to prevent, treat or alleviate the symptoms associated with a condition which would benefit from an increase in GLP-1, such as, for example, diabetes. In another example, when the therapeutic protein is an interleukin, the present disclosure provides using the recombinant bacterium to prevent, treat or alleviate the symptoms associated with a condition which would benefit from an increase in interleukin, such as, for example, an inflammatory condition.

In an embodiment in which the heterologous protein encoded by the genetic cargo is a programmable nuclease, the recipient bacterium can be modified to express one or more of a TALEN, a zinc finger nuclease or a Cas protein. The expression of the programmable nuclease is intended to provide a therapeutic benefit to the subject having received the recombinant bacterium and being afflicted by the recipient bacterium. The administration of the conjugative bacterial cell recombinant bacteria can, for example, kill the recipient bacterium, sensitize the recipient bacterium to an antibiotic, or modify the recipient bacterium in order to suppress the expression of a protein, or a non-coding RNA, contributing to the pathogenicity. When the heterologous protein is a Cas protein, like a Cas9 protein, the present disclosure provides using the conjugative bacterial cell recombinant bacterium to prevent, treat or alleviate the symptoms associated with an infection or a dysbiosis caused by the intended recipient bacterium. In an embodiment, the infection and/or dysbiosis caused by the intended recipient bacterium is located in the gastro-intestinal tract and the recombinant bacterium is administered to prevent, treat or alleviate the symptoms of such infection and/or dysbiosis. For example, when the subject is infected with a multidrug resistant shiga toxin-producing E. coli, the recombinant bacterium can be used to restore drug sensitivity in the recipient bacterium and/or inhibit the expression of the shiga toxin. In yet another example, when the subject is afflicted by an adherent-invasive E. coli and is also afflicted by Crohn's disease or an inflammatory bowel disease linked to a dysbiosis, the recombinant bacterium can be used to inhibit the expression of an adhesion pilus to render the recipient bacterium less adherent to the gastro-intestinal wall and, in some embodiments, treat the dysbiosis. In still another example, when the subject is afflicted with a urinary tract infection or a blood septicemia linked to a dysbiosis, the recombinant bacterium can be used to inhibit the expression of an adhesion pilus or a virulence factor to render to recipient bacterium less virulent and, in some embodiments, treat the dysbiosis.

In an embodiment in which the genetic cargo encodes one or more non-coding RNA, the recipient bacterium can subsequently express one or more non-coding RNA. For example, the recipient bacterium can express one or more crRNA, and/or tracrRNA, and/or anti-sense RNA, and/or gRNA, and/or rRNA, and/or tRNA. The expression of the non-coding RNA is intended to provide a therapeutic benefit to the subject having received the recombinant bacterium. For example, when the therapeutic non-coding RNA is an antisens-RNA, it can knock down the expression of a virulence factor thus rendering the recipient unable to infect the subject.

In an embodiment in which the genetic cargo encodes one or more non-coding RNA and one or more heterologous proteins, the bacterium receiving the genetic cargo can subsequently express one or more non-coding RNA and one or more heterologous proteins. For example, the recipient bacterium can express one or more crRNA, and one or more Cas proteins. The expression of the crRNA and Cas protein is intended to provide a therapeutic benefit to the subject having received the conjugative bacterial cell recombinant bacterium. For example, the simultaneous presence of crRNA and Cas9 at specific loci in the recipient bacterium's genome will result in double-strand cleavage at those sites. These cuts will subsequently induce the death of the recipient bacterium.

The recombinant bacterium can optionally be used in combination with an antibiotic. Examples of antibiotics include, without limitation, aminoglycosides, ansamycins, carbapenems, cephalosporins, glycopeptides, lincosamides, lipopeptides, macrolides, monobactams, nitrofurans, oxazolidonones, penicillins, quinolones, sulfonamides, tetracyclines, and combinations thereof. Examples of aminoglycosides include, without limitation, amikacin, gentamicin, kanamycin, neomycin, netilmicin, tobramycin, paromomycin, spectinomycin and combinations thereofs. Examples of ansamycins include, without limitation, geldanamycin, herbimycin, rifaximin (streptomycin) and combinations thereof. Examples of carbapenems include, without limitation, ertapenem, doripenem, imipenem/cilastatina, Meropenem and combinations thereof. Examples of cephalosporins include, without limitation, cefadroxil, cefazolin, cefalotin or cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole and combinations thereof. Examples of glycopeptides include, without limitation, teicoplanin, vancomycin, telavancin and combinations thereof. Examples of lincosamides include, without limitation, clindamycin, lincomycin and combinations thereof. An example of a lipopeptide includes, without limitation, daptomycin. Examples of macrolides include, without limitation, azithromycin, clarithromycin, dirithromycin, erythromycin, roxithromycin, troleandomycin, telithromycin, spiramycin and combinations thereof. An example of a monobactams includes, without limitation, aztreonam. Examples of nitrofurans include, without limitation, furazolidone, nitrofurantoin and combinations thereof. Examples of oxazolidonones include, without limitation, linezolid, posizolid, radezolid, orezolid and combinations thereof. Examples of penicillins include, without limitation, amoxicillin, ampicillin, azlocillin, carbenicillin, cloxacillin, dicloxaciUin, flucloxaciUin, mezlocillin, methicillin, nafcillin, oxacillin, penicillin G, penicillin V, piperacillin, penicillin G, temocillin, ticarcillin and combinations thereof. Examples of quinolones include, without limitation, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin and combinations thereof. Examples of sulfonamides include, without limitation, mafenide, sulfacetamide, sulfadiazine, silver sulfadiazine, sulfadimethoxine, sulfamethizole, sulfamethoxazole, sulfanamide (archaic), sulfasalazine, sulfisoxazole, trimethoprim-sulfamethoxazole(Co-trimoxazole) (TMP-SMX), sulfonamidochrysoidine(archaic) and combinations thereof. Examples of tetracyclines include, without limitation, demeclocycline, doxycycline, minocycline, oxytetracycline, tetracycline and combinations thereof.

The present invention will be more readily understood by referring to the following examples which are given to illustrate the invention rather than to limit its scope.

### EXAMPLE I—IN VITRO BACTERIAL CONJUGATIVE TRANSFER EFFICIENCY IS NOT PREDICTIVE OF IN VIVO BACTERIAL CONJUGATIVE TRANSFER EFFICIENCY

Strains, plasmids and growth conditions. All strains and plasmids used in this Example are described in Table 1. All oligonucleotide sequences are provided in Table 2. Cells were typically grown in Luria broth Miller (LB) or on Luria broth agar Miller medium supplemented, when needed, with antibiotics at the following concentrations: ampicillin (Ap) 100 μg/mL, chloramphenicol (Cm) 34 μg/mL, kanamycin (Km) 50 μg/mL, nalidixic acid (Nx) 4 μg/mL, spectinomycin (Sp) 100 μg/mL, streptomycin (Sm) 50 μg/mL, sulfamethoxazole (Su) 160 μg/mL, tetracycline (Tc) 15 μg/mL, and trimethoprim (Tm) 32 μg/mL. Diaminopimelic acid (DAP) auxotrophy was complemented by adding DAP at a final concentration of 57 μg/mL in the medium. All cultures were routinely grown at 37° C. Cells with thermosensitive plasmids (pSIM6, pCP20, pGRG36) were grown at 30° C. No bacterial cultures over 18 hours of age were used in the experiments.

TABLE 1
List of strains and plasmids used in the Examples.

| | | |
|---|---|---|
| Strain or plasmid | Relevent phenotype or genotype | Source/Reference |

Citrobacter rodentium

| | | |
|---|---|---|
| DBS100 | Murine enteric pathogen | ATCC 51459 |
| KN04 | DBS100, Sm^{R}, Cm^{R} | Example III |

Enterobacter aerogenes

| | | |
|---|---|---|
| ATCC 35029 | Synonym Klebsiella aerogenes, opportunistic pathogen | ATCC 35029 |

E. coli

| | | |
|---|---|---|
| BW25113 | F⁻, DE(araD-araB)567, lacZ4787(del)::rrnB-3, LAM⁻, | CGSC#: 7636 |
| | rph-1, DE(rhaD-rhaB)568, hsdR514 | |
| EC100Dpir+ | F− mcrA Δ(mrr-hsdRMS-mcrBC) ϕ80dlacZΔM15 | #ECP09500 (Lucigen) |
| | ΔlacX74 recA1 endA1 araD139 Δ(ara, leu)7697 galU | |
| | galK λ− rpsL nupG pir+ (DHFR) | |
| K-12 J53 | F⁺ met pro | DSM-4246 (DSMZ) |
| KN01 | Sm^{R}Sp^{R} Nissle 1917 | Example I |
| KN01ΔdapA | ΔdapA KN01 | Example I |
| KN02 | Sm^{R}Cm^{R} Nissle 1917 | Example I |
| KN03 | Sm^{R}Tc^{R} Nissle 1917 | Example I |
| KN05 | pir+ Nissle 1917 | Example III |
| MFDpir+ | MG1655 RP4-2-Tc::[ΔMu1::aac(3)IV-ΔaphA-Δnic35- | (Ferrière et al. 2010) |
| | ΔMu2::zeo] ΔdapA::(erm-pir) ΔrecA | |
| MG1655Nx^{R} | K-12 F− λ− ilvG− rfb-50 rph-1 Nx^{R} | (Ceccarelli et al. 2008) |
| MG1655Rf^{R} | K-12 F− λ− ilvG− rfb-50 rph-1 Rf^{R} | (Ceccarelli et al. 2008) |
| Nissle 1917 | Wildtype probiotic strain | DSM-6601 (DSMZ) |

Salmonella typhimurium

| | | |
|---|---|---|
| SR-11 | High virulence murine pathogen | (Benjamin et al. 1986) |

Plasmid

| | | |
|---|---|---|
| Kill1 Cargo insertion device (SEQ | oriV_{pSC101ts}, attP_{Bxb1}, FRT, 1 gRNA vs cat, aph-IIIa (Km^{R}), | Example III |
| ID NO: 143) | cas9 | |
| Kill3 Cargo insertion device (SEQ | oriV_{pSC101ts}, attP_{Bxb1}, FRT, 3 gRNA vs cat, aph-IIIa (Km^{R}), | Example III |
| ID NO: 144) | cas9 | |
| pBXB1 (SEQ ID NO: 145) | oriV_{pMB1}, bxb1 integrase, bla (Ap^{R}) | Example III |
| pCas9 | oriV_{pSC101ts}, flp, Ap^{R} | E. coli Genetic Stock Center |
| pE-FLP | oriV_{R101}, tetB, araC | Rodrigue Laboratory stock |
| pFG036 (SEQ ID NO: 146) | cl, tetM, oriV_{ColE1} | Example II |
| pFG051(SEQ ID NO: 147) | aad7, cat, Tn5 tnp, oriV R6K, oriT_{RP4} | Example II |
| pGRG36 | oriV_{pSC101ts}, oriT_{RP4}, insertion machinery, araC, Ap^{R} | Addgene #16666 |
| pGRG36-pir (SEQ ID NO: 148) | pGRG36 with pir insert | Example III |
| pGRG36-SmCm (SEQ ID NO: | pGRG36 with Sm^{R}Cm^{R} insert | Example I |
| 149) | | |
| pGRG36-SmSp (SEQ ID NO: | pGRG36 with Sm^{R}Sp^{R} insert | Example I |
| 150) | | |
| pGRG36-SmTc (SEQ ID NO: | pGRG36 with Sm^{R}Tc^{R} insert | Example I |
| 151) | | |
| pKD3 | oriV_{R6K}, FRT flanked Cm^{R}, Ap^{R} | E. coli Genetic Stock Center |
| pKD4 | oriV_{R6K}, FRT flanked Km^{R}, Ap^{R} | E. coli Genetic Stock Center |
| pKN02 (SEQ ID NO: 152) | bla, oriT_{RP4}, oriV_{R6K}, cas9, gRNA 1 | Example III |
| pKN30 (SEQ ID NO: 153) | oriV_{pBBR1}, aph-III, oriT_{TP114} | Example III |
| pKN31 (SEQ ID NO: 154) | oriV_{pBBR1}, aph-III, oriT_{TP114}Δnicking site | Example III |
| pNA01(SEQ ID NO: 155) | oriV_{pBBR1}, tetB, oriT_{TP114} | Example III |
| pNA02 (SEQ ID NO: 156) | oriVp_{pBBR1}, tetB, oriT_{TP114}Δnicking site | Example III |
| pNA22 (SEQ ID NO: 157) | oriV_{R6K}, cat, TP114 repA + 1,000 bp | Example III |
| pNA23 (SEQ ID NO: 158) | oriV_{R6K}, cat, 1,000 bp + TP114 repA | Example III |
| pNA24 (SEQ ID NO: 159) | oriV_{R6K}, cat, 1,000 bp + TP114 repA + 1,000 bp | Example III |
| pOSIP-TT | oriV_{R6K}, attB_{Bxb1}, tetB (Tc^{R}), FRT | Example III |
| pOSIP-TT | oriV_{pMB1}, bla, Biobrick | PMID: 21601683 |
| pPilS | oriV_{p15A}, cat, araC, P_{BAD}, pilS | Example II |
| pPilV4′ | oriV_{p15A}, cat, araC, P_{BAD}, pilV4′ | Example II |
| pRAD module G8-C1 | oriV_{pMB1}, cat, Biobrick | PMID: 21601683 |
| pREC1 (SEQ ID NO: 160) | oriV_{pSC101ts}, Lambda Red recombinase, Ap^{R} | PMID: 16750601 |
| pSB1A3 | oriV_{pBBR1}, Lambda Red recombinase, Cm^{R} | PMID: 16750601 |
| PSB1C3 | oriV_{pMB1}, araC controlled GFP, cat (Cm^{R}) | BBa_I746908 (IGEM) |
| pSIM6 | IPTG inducible expression plasmid | V36020 (THERMOFISHER) |
| pSIM7 | Conjugative plasmid | MF521836.1 |
| pT | TP114::tetB-Kill1 after FRT driven deletion of tetB and | Example III |
| | oriV_{pSC101ts}, | |
| pTRC-HisB | TP114::tetB-Kill3 after FRT driven deletion of tetB and | Example III |
| | oriV_{pSC101ts} | |
| TP114 (SEQ ID NO: 161) | TP114Δaph-III::tetB | Example III |
| TP114::Kill1(SEQ ID NO: 164) | TP114::tetB with Inserted Kill1 insertion device | Example III |
| TP114::Kill3 (SEQ ID NO: 165) | TP114::tetB with inserted Kill 3 insertion device | Example III |
| TP114::tetB (SEQ ID NO: 166) | TP114::tetB with oriT nicking site deletion | Example III |
| TP114::tetB-Kill1 (SEQ ID NO: | TP114ΔpilS::cat | Example II |
| 168) | | |
| TP114::tetB-Kill3 (SEQ ID NO: | TP114pilVΔshufflon-rci ::cat (C-terminal portion of pilV | Example II |
| 169) | replaced by a flag-tag) (SEQ ID NO: 170) | |
| TP114ΔoriT::cat-tetB | TP114ΔrepA::cat-oriV_{R6K} | Example III |
| (SEQ ID NO: 167) | | |
| TP114Δrci | TP114Δrcideletion mutant | Example II |
| TP114ΔpilV-rci | TP114ΔpilV-shufflon-rci deletion mutant | Example II |
| TP114pilVΔshufflon-rci | TP114pilVΔshufflon-rci replace by FLAG-tag | Example II |
| TP114Δshufflon::pilV1-cat | Locked pilV variant 1 | Example II |
| TP114Δshufflon::pilV2-cat | Locked pilV variant 2 | Example II |
| TP114Δshufflon::pilV3-cat | Locked pilV variant 3 | Example II |
| TP114Δshufflon::pilV3′-cat | Locked pilV variant 3′ | Example II |
| TP114Δshufflon::pilV4-cat | Locked pilV variant 4 | Example II |
| TP114Δshufflon::pilV4′-cat | Locked pilV variant 4′ | Example II |
| TP114Δshufflon::pilV5-cat | Locked pilV variant 5 | Example II |
| TP114Δshufflon::pilV5′-cat | Locked pilV variant 5′ | Example II |

DNA Manipulations.

A detailed list of oligonucleotide sequences used in this Example is found in Table 2. Plasmids were prepared using EZ10-Spin Column Plasmid Miniprep kit (BIOBASIC #BS614) whereas genomic DNA (gDNA) minipreps were prepared using Quick gDNA miniprep (ZYMO RESEARCH) according to the manufacturer's instructions. PCR amplifications were performed using Veraseq DNA polymerase (Enzymatics) or TaqB (Enzymatics) for DNA parts amplification and screening respectively. Digestion with restriction enzymes were incubated for 1 hour at 37° C. following manufacturer's recommendations. Plasmids were assembled by Gibson assembly using the NEBuilder Gibson Assembly mix (NEB) following manufacturer's protocol.

Recombineering.

All recombineering experiments were performed using pSIM6 as described previously (PMID: 16750601). Briefly, the E. coli strain containing pSIM6 was cultured at 30° C. until an optical density of 0.4 to 0.8 at 600 nanometers was reached. Then, the cells were heat-shocked for 15 minutes at 42° C., washed and the recombineering cassette was electroporated in the heat shocked E. coli cells. Cells were then incubated overnight at room temperature before plating on selective medium. The colonies were then screened by PCR to identify positive clones.

TABLE 2
Description of the oligonucleotides used in the Examples

| Purpose | Name | Sequence^{a,b} | Template | To amplify |
|---|---|---|---|---|
| pGRG_SmSp | oGSS1- | GGATCCTAGTAAGCCACGTTTTAATTAATCAGATCCCGGG | gBlock_16S_ | 16S tag |
| | F | CCTACGGGAGGCAGCAGTGG | tagR | |
| | | (SEQ ID NO: 29) | | |
| | oGSS1- | CGGGGAACTAGGAGGGTATGGTGCGCGCATGGAAA | | |
| | R | GACTACCAGGGTATCTAATCCTGTT | | |
| | | (SEQ ID NO: 30) | | |
| | oGSS2- | CGCACCATACCCTCCTAGTTCCCCGGTTATCTCTC | pFG051 | aad7 |
| | F | CTGTCTCTTATACACATCTGACGCT | (unpublished) | |
| | | (SEQ ID NO: 31) | | |
| | oGSS2- | GGGGTCGACGCGGCCGTGGCGCGCCTCCTAGGTGCTCGAG | | |
| | R | GCAAGCGAACCGGAATTGCC | | |
| | | (SEQ ID NO: 32) | | |
| pGRG_SmCm | oGSC1- | GATCCTAGTAAGCCACGTTTTAATTAATCAGATCCCGGGCTA | SXT | strA-strB |
| | F | GTATGACGTCTGTCGCAC | | |
| | | (SEQ ID NO: 33) | | |
| | oGSC1- | TGCTAGCTTTGAAAATTAAGAGGTATATATTATTGAATCGAAC | | |
| | R1 | TAATATTTTTTTTGGTG | | |
| | | (SEQ ID NO: 34) | | |
| | oGSC1- | GTGTCAACGTTTACAGCTAGCTCAGTCCTAGGTATTATGCTA | | Add P1-U8 |
| | R2 | GCTTTGAAAATTAAGAGG | | |
| | | (SEQ ID NO: 35) | | |
| | oGSC2- | TACCTAGGACTGAGCTAGCTGTAAA | pTRC-HTsB | lacl-P_{trc} |
| | F1 | CGTTGACACCATCGAATGGTGCAAAACCTTTCGCG | | |
| | | (SEQ ID NO: 36) | | |
| | oGSC2- | ATATCCCGAATGTGCAGTTAACGA | | |
| | F2 | CGTTGACACCATCGAATGGTGCAAAACCTTTCGCGG | | |
| | | (SEQ ID NO: 37) | | |
| | oGSC2- | TAATATATACCTCTTTAATTTTTAATAATAAAGTTAATCG | | |
| | R | (SEQ ID NO: 38) | | |
| | oGSC3- | TATTATTAAAAATTAAAGAGGTATATATTA | gBlock_ | NeonGreen |
| | F | ATGGTTTCTAAAGGAGAAGAAAAAAATATG | NeonGreen | |
| | | (SEQ ID NO: 39) | | |
| | oGSC3- | CCTCTTTCCACTGCTGCCTCCCGTAGGTTA | | |
| | R | TTTATATAATTCATCCATTQCCATAACATC | | |
| | | (SEQ ID NO: 40) | | |
| | oGSC4- | AGCATTTACAGATGTTATGGGAATGGATGAATTATATAAATAA | gBlock_16S_ | 16S tag |
| | F | CCTACGGGAGGCAGCAG | tagD | |
| | | (SEQ ID NO: 41) | | |
| | oGSC4- | ACCTCTTACGTGCCCGATCAACTCGAGGCATGCCTGCAG | | |
| | R | GACTACCAGGGTATCTAATCC | | |
| | | (SEQ ID NO: 42) | | |
| | oGSC5- | GAACAGGATTAGATACCCTGGTAGTCCTGCAGGCATGC | pSB1C3 | cat |
| | F | CTCGAGTTGATCGGGCACGTAA | | |
| | | (SEQ ID NO: 43) | | |
| | oGSC5- | GGTCGACGCGGCCGTGGCGCGCCTCCTAGGTGCTCGAG | | |
| | R | CTCGAGGCTTGGATTCTCACCA | | |
| | | (SEQ ID NO: 44) | | |
| | oGSC6 | GAGCTTGGCGTAATCATGGTCATAGCTGTTTCCTGTGTGA | PGRG36 | Screen |
| | -F | CGCTTAATGCGCCGCTACAG | bacbone | plasmid |
| | | (SEQ ID NO: 93) | | |
| | oGSC6- | CTGGTGAAAGTAAAAGATGCTGAAGATCAGTTGGGTGCAC | | presence |
| | R | CGCGCAATTAACCCTCACTA | | |
| | | (SEQ ID NO: 94) | | |
| pGRG_SmTc | oGST1- | GATCCTAGTAAGCCACGTTTTAATTAATCAGATCCCGGG | SXT | strA-strB |
| | F | CTAGTATGACGTCTGTCGCAC | | |
| | | (SEQ ID NO: 23) | | |
| | oGST1- | GTAGGTTATTTATATAATTCATCCATTCCCATAACATCTG | | |
| | R | TTTACAGCTAGCTCAGTCCT | | |
| | | (SEQ ID NO: 24) | | |
| | oGST2- | AAGCTAGCATAATACCTAGGACTGAGCTAGCTGTAAA | gBlock_16S_ | 16S tag |
| | F | CAGATGTTATGGGAATGGATGAA | tagD | |
| | | (SEQ ID NO: 25) | | |
| | oGST2- | CCCCAAAACTTTCCCCAAAACCCTTCCCCAAAACTGGCTAT | | |
| | R | ACTCGAGGCATGCCTGCAG | | |
| | | (SEQ ID NO: 26) | | |
| | oGST3- | GGATTAGATACCCTGGTAGTCCTGCAGGCATGCCTCGAGT | pFG018 | tetB |
| | F | ATAGCCAGTTTTGGGGAAGG | | |
| | | (SEQ ID NO: 27) | | |
| | oGST3- | GGGGTCGACGCGGCCGTGGCGCGCCTCCTAGGTGCTCGAG | | |
| | R | CTAGGTCGACGCTTGGATTC | | |
| | | (SEQ ID NO: 28) | | |
| dap A | oDTD1- | CGCGTGCCTCGGCAAAATGCCCTTCTGCTGCCAGTTTGCA | pKD4 | aph-IIIa |
| deletTon | F | GTGTAGGCTGGAGCTGCTTC | | |
| | | (SEQ ID NO: 45) | | |
| | oDTD1- | AAACGTACCATTGAGACACTTGTTTGCACAGAGGATGGCCC | | |
| | R | TGGGAATTAGCCATGGTCC | | |
| | | (SEQ ID NO: 46) | | |
| | oDTD2 | CA*A*A*TAGTTTGTTGTGTAATGGCATCAGACGCTGATTA | PrevTous | Add |
| | -F | ATAACGCGTGCCTCGGCAAAAT | | homology |
| | | (SEQ ID NO: 47) | | |
| | oDTD2 | AC*G*G*TTCTGTCTGCTTGCTTTTAATGCCATACCA | | |
| | -R | AACGTACCATTGAGACACTTGTTTGC | | |
| | | (SEQ ID NO: 48) | | |
| | oDTD3 | CCGTTTCTGCGGACTGGCTT | pKD4 | Screen dapA |
| | -int | (SED ID NO: 49) | | deletion |
| | oDTD2 | TAGCGGCTATCACCAACATC (SEQ ID NO: 50) | Nissle | Screen dapA |
| | 3-F | | 1917 | deletion |
| | oDTD3 | GTGAAGCGCCTTATGAACAATG (SEQ ID NO: 51) | | |
| | -R | | | |
| TP114 | oTPS1 | TGCTGAACCAGTAACAACCACC (SEQ ID NO: 67) | TP1141 bp | Sanger |
| sanger | -F | | sanger | sequencing |
| sequencing | | | | |
| | oTPS1 | GTCGCCGCTGTGGATTCAAC (SEQ ID NO: 68) | | TP114 |
| | -R | | | |
| | oTPS2 | GTTCAATACACATTACAGCCCACC (SEQ ID NO: 69) | TP11410 | Sanger |
| | -F | | kb sanger | sequencing |
| | oTPS2 | CTGCGCTCAAAGTCACGTATGG (SEQ ID NO: 70) | | TP114 |
| | -R | | | |
| | oTPS3 | TTACGCAACAGAATCTGAAAGCAC (SEQ ID NO: 71) | TP114 20 | Sanger |
| | -F | | kb sanger | sequencing |
| | oTPS3 | GAAGGTGGCCTGTCATCGAG (SEQ ID NO: 72) | | TP114 |
| | -R | | | |
| | oTPS4 | TGTCCGATTCGTCCTGGTTG (SEQ ID NO: 73) | TP114 25 | Sanger |
| | -F | | kb sanger | sequencing |
| | oTPS4 | GTATTTGTCCAGCGCCCGG (SEQ ID NO: 74) | | TP114 |
| | -R | | | |
| | oTPS5 | TTCAGATGCGTCGTGCAATG (SEQ ID NO: 75) | TP114 37 | Sanger |
| | -F | | kb sanger | sequencing |
| | oTPS5 | CACACTTGAGCGTCTTTCTGA (SEQ ID NO: 76) | | TP114 |
| | -R | | | |
| | oTPS6 | AGAAGCTCTTGAGTCCGACC (SEQ ID NO: 77) | TP114 42.5 | Sanger |
| | -F | | kb sanger | sequencing |
| | oTPS6 | GACTTATTCCGCCAACCCAAATT (SEQ ID NO: 78) | | TP114 |
| | -R | | | |
| | oTPS7 | GGCCCGCTCAAGGTCTTTC (SEQ ID NO: 79) | TP114 47 | Sanger |
| | -F | | kb sanger | sequencing |
| | oTPS7 | GCTGGAGAACACCCTGATTATGT (SEQ ID NO: 80) | | TP114 |
| | -R | | | |
| | oTPS8 | AAAGTTCTTTGCGCCTGTCATAGC (SEQ ID NO: 81) | TP114 50 | Sanger |
| | -F | | kb sanger | sequencing |
| | oTPS8 | GAAGCCAGGTTTGTTGCTGTG (SEQ ID NO: 82) | | TP114 |
| | -R | | | |
| | oTPS9 | TTTCTCTGCTACAGCATCTTTCTTC | TP114 52.5 | Sanger |
| | -F | (SEQ ID NO: 83) | kb sanger | sequencing |
| | oTPS9 | GGAACTGCCTCGGTGAAT (SEQ ID NO: 84) | | TP114 |
| | -R | | | |
| | oTPS10 | GGCATAAGGCGTGGACAATGG (SEQ ID NO: 85) | TP114 57.5 | Sanger |
| | 0-F | | kb sanger | sequencing |
| | oTPS10 | CAAACGTGCTAATCGCCTGGC (SEQ ID NO: 86) | | TP114 |
| | 0-R | | | |
| pBXB1 | oBXB1 | ATTTCCCCGAAAAGTGCCACCTGACGTCTAAGAAACCATT | gBIock- | bxb1 |
| | -F | CACGAGGCAGAATTTCAGAT (SEQ ID NO: 1) | Bxb1 | integrase |
| | oBXB1 | ATCCCCTGATTCTGTGGATAACCGTATTACCGCCTTTGAG | | |
| | -R | CGCCCTGCAGGAAATAATAA(SEQ ID NO: 2) | pSB1A3 | bla-oriV_{pMB1} |
| | oBXB2 | GATTATTAATCCGGCTTTTTTATTATTTCCTGCAGGGCG | | |
| | -F | CTCAAAGGCGGTAATACGGTT (SEQ ID NO: 3) | | |
| | oBXB2 | AAGCTAAGGATTTTTTTTATCTGAAATTCTGCCTCGTG | | |
| | -R | AATGGTTTCTTAGACGTCAGGT (SEQ ID NO: 4) | | |
| Kill3 | oKIL1- | AACCACCGCGGTCTCAGTGGTGTACGGTACAAACCCCGAC | pGRG36 | OriV_{pSc101ts} |
| Cargo | F | CGACAGTAAGACGGGTAAGC (SEQ ID NO: 5) | | |
| insertion | | | | |
| device | | | | |
| | oKIL1- | TATACTTTCTAGAGAATAGGAACTTCGGAATAGGAACTTC | | |
| | R | GGCTGAAAGCGCTATTTCTT (SEQ ID NO: 6) | | |
| | oKIL2- | AAGTTCCTATTCTCTAGAAAGTATAGGAACTTCGTG | pKNO2 | gRNA 1 |
| | F | CACTGATTAAGCATTGGTAACAGG | (unpub- | |
| | | (SEQ ID NO: 7) | lished) | |
| | oKIL2- | TTTCGGGACATTCAGGAGATTTTCGCCGGACGTACGCATT | | |
| | R | TTCAGCACACTGAGACTTGT (SEQ ID NO: 8) | | |
| | oKIL3- | AATGCGTACGTCCGGCGAAAATCTCCTGAATGTCCCGAAA | pKN02 | gRNA2 |
| | F | GGGCAGAAAGATGAATGACT (SEQ ID NO: 9) | (unpub- | |
| | | | lished) | |
| | oKIL3- | CTAAAACAGGGATTGGCTGAGACGAAA | | |
| | R | GAATCTATTATACAGAAAAATTTTCCTGAAAGC | | |
| | | (SEQ ID NO: 10) | | |
| | oKIL4- | TTCTGTATAATAGATTCTTTCGTCTCAGCCAATCCCT | pKNO2 | gRNA 2 |
| | F | GTTTTAGAGCTAGAAATAGCAAG | (unpub- | |
| | | (SEQ ID NO: 11) | lished) | |
| | oKIL4- | AACCGAGTGACCAAGAGAGGATGAAGCATTTGCCGAGTAG | | |
| | R | TTCAGCACACTGAGACTTGT (SEQ ID NO: 12) | | |
| | oKIL5- | CTACTCGGCAAATGCTTCATCCTCTCTTGGTCACTCGGTT | pKN02 | gRNA 3 |
| | F | GGGCAGAAAGATGAATGACTGTC (SEQ ID NO: 13) | (unpub- | |
| | | | lished) | |
| | oKIL5- | CTAAAACTATTGGCCACGTTTAAATCA | | |
| | R | GAATCTATTATACAGAAAAATTTTCCTGAAAGC | | |
| | | (SEQ ID NO: 14) | | |
| | oKIL6- | TTCTGTATAATAGATTCTGATTTAAACGTGGCCAATA | pKN02 | gRNA 3 |
| | F | GTTTTAGAGCTAGAAATAGCAAG | (unpub- | |
| | | (SEQ ID NO: 15) | lished) | |
| | oKIL6- | GTCGGAAAAGTGGCCATCATTTGACGAACTACAGCCCGGG | | |
| | R | TTCAGCACACTGAGACTTGT (SEQ ID NO: 16) | | |
| | oKIL7- | CCCGGGCTGTAGTTCGTCAAATGATGGCCACTTTTCCGACG | pKD4 | aph-IIIa |
| | F | GTGCTGACCCCGGATGAAT (SEQ ID NO: 17) | | |
| | oKIL7- | ATCATTCTATAGTATTAAGTATTGTTTTATGGCTGATAAA | | |
| | R | AGCGCTTTTGAAGCTGGGGT (SEQ ID NO: 18) | | |
| | oKIL8- | GTTCTTCGCCCACCCCAGCTTCAAAAGCGCT | pKN02 | cas9 |
| | F | TTTATCAGCCATAAAACAATACTTAATAC | | |
| | | (SEQ ID NO: 19) | | |
| | oKIL8- | CACCACTGAGACCGCGGTGGTTGACCAGACAAACCACGAC | | |
| | R | TCAGTCACCTCCTAGCTGAC (SEQ ID NO: 20) | | |
| Kill1 | oKIL9- | AAGTTCCTATTCTCTAGAAAGTATAGGAACTTCGTG | pKN02 | gRNA 1 |
| Cargo | F | CACTGATTAAGCATTGGTAACAGG | (unpub- | |
| insertion | | (SEQ ID NO: 21) | lished) | |
| device | | | | |
| | oKIL9- | GTCGGAAAAGTGGCCATCATTTGACGAACTACAGCCCGGG | | |
| | R | TTCAGCACACTGAGACTTGT (SEQ ID NO: 22) | | |
| pREC1 | oREC1 | GACGACGGCGGTCTCCGTCGTCAGGATCATCCGGGCGGA | pKD4 | oriV_{R6K} |
| | -F | GGATATTCATATGGACCATGG (SEQ ID NO: 52) | | |
| | oREC1 | TATACTTTCTAGAGAATAGGAACTTCGGAATAGGAACTTC | | |
| | -R | TTTTGCGGCCGCAAGATCCG (SEQ ID NO: 53) | | |
| | oREC2 | TATTCCGAAGTTCCTATTCTCTAGAAAGTATAGGAACTTC | pFG018 | tetB |
| | -F | ATAGCCAGTTTTGGGGAAGG (SEQ ID NO: 54) | | |
| | oREC2 | TCCTGACGACGGAGACCGCCGTCGTCGACAAGCCGGCCGA | | |
| | -R | CTAGGTCGACGCTTGGATTC (SEQ ID NO: 55) | | |
| TP114:: | oTPT1- | ACATGGCAAAGGTAGCGTTGCCAATGATGTTACAGATGAG | pREC1 | FRT-tetB- |
| tetB | F | CGGATCTTGCGGCCGCAAAA (SEQ ID NO: 56) | | att_{Bxb1} |
| | oTPT1- | GCCGATGCGCCAACCGCATTCATTAAAGACTAACTA | | |
| | R | CCATGGTCCATATGAATATCCTCC (SEQ ID NO: 57) | | |
| | oTPT2- | GAGCCATATTCAACGGGAAACGTC (SEQ ID NO: 58) | TP114:: | Screen tetB |
| | F | | tetB | insertion |
| | oTPT2- | CACATCGGTGAAAGCTATGCC (SEQ ID NO: 59) | | |
| | R | | | |
| Screen | oTPK1 | GCTCGCTTGGACTCCTGTTG (SEQ ID NO: 60) | pREC1 | screen:: |
| TP114::Kill | -F | | and | tetB in |
| | oTPK1 | CGTTGGCAAGACTGGCATGAT (SEQ ID NO: 61) | | |
| | -R | | | |
| | oTPK2 | TTGAAGGGTAGTCCAGAAGATAACG | TP114:: | Screen:: |
| | -F | (SEQ ID NO: 62) | Kill3 | Kill3/ |
| | | | or 1 | Kill1 |
| | | | | TP114 |
| | oTPK2 | GGTAAATGGCACTACAGGCGC (SEQ ID NO: 63) | | |
| | -R | | | |
| | oTPK3 | CCTGTTACCAATGCTTAATCAGTGCAC | TP114:: | Screen FRT |
| | | (SEQ ID NO: 64) | Kill3 | recombination |
| | | | or 1 | |
| | oTPK4 | GTGCACTGATTAAGCATTGGTAACAGG | TP114:: | Screen ARNg |
| | -F | (SEQ ID NO: 65) | Kill3 | number |
| | | | or 1 | |
| | oTPK4 | GGTCAGCACCGTCGGAAAAG (SEQ ID NO: 66) | | |
| | -R | | | |
| pNA22 | oNA1- | ATTACACGTCTTGAGCGATTCGCG | TP114 | repA + 1,000 |
| | F | ACTATCACCGGAAGAGCAGA (SEQ ID NO: 95) | | bp |
| | oNA1- | GAAGCAGCTCCAGCCTACACTCGAG | | |
| | R | TTTATTCCGCAAGTGATTAA (SEQ ID NO: 96) | | |
| | oNA2- | TTAATCACTTGCGGAATAAACTCGA | pKD3 | pKD3's |
| | F | GTGTAGGCTGGAGCTGCTTC (SEQ ID NO: 97) | | backbone |
| | oNA2- | TCTGCTCTTCCGGTGATAGTCGCG | | |
| | R | AATCGCTCAAGACGTGTAAT (SEQ ID NO: 98) | | |
| pNA23 | oNA3- | ATTACACGTCTTGAGCGATTCGCG | TP114 | 1,000 bp |
| | F | ATCCCCGGAATACAGCGTCAT (SEQ ID NO: 99) | | repA |
| | oNA3- | GAAGCAGCTCCAGCCTACACGAATT | | |
| | R | CTAGTGGGGTGGCGAAGCTG (SEQ ID NO: 100) | | |
| | oNA4- | CAGCTTCGCCACCCCACTAGAATTC | pKD3 | pKD3's |
| | F | GTGTAGGCTGGAGCTGCTTC (SEQ ID NO: 101) | | backbone |
| | oNA4- | ATGACGCTGTATTCCGGGGATCGCG | | |
| | R | AATCGCTCAAGACGTGTAAT (SEQ ID NO: 102) | | |
| pNA24 | oNA3- | ATTACACGTCTTGAGCGATTCGCG | TP114 | 1,000 bp |
| | F | ATCCCCGGAATACAGCGTCAT (SEQ ID NO: 103) | | repA + 1,000 |
| | oNA1- | GAAGCAGCTCCAGCCTACACTCGAG | | bp |
| | R | TTTATTCCGCAAGTGATTAA (SEQ ID NO: 104) | | |
| | oNA2- | TTAATCACTTGCGGAATAAACTCGA | pKD3 | pKD3's |
| | F | GTGTAGGCTGGAGCTGCTTC (SEQ ID NO: 105) | | backbone |
| | oNA4- | ATGACGCTGTATTCCGGGGATCGCG | | |
| | R | AATCGCTCAAGACGTGTAAT (SEQ ID NO: 106) | | |
| pGRG-pir⁺ | opir1-F | CCTAGTAAGCCACGTTTTAATTAATCAGATCCCGGG | ECWoDpir⁺ | pir |
| | | CATGAGTGGATAGTACGTTGCTAA | | |
| | | (SEQ ID NO: 107) | | |
| | opir1-R | GTCAGTTTAGGTTAGGCGCCATGCATCTCGAGGCTTGG | | |
| | | TCACCCCTTAGCTTTTTTGGGA (SEQ ID NO: 108) | | |
| | opir2-F | CCAAGCCTCGAGATGCATGG (SEQ ID NO: 109) | pFG018 | rrnB |
| | | | | terminator |
| | opir2-R | CGCGGCCGTGGCGCGCCTCCTAGGTGCTCGAG | | |
| | | GGACGTCTAAGAAACCATTATTATCATG | | |
| | | (SEQ ID NO: 110) | | |
| | opir3-F | GATGCTGGTGGCGAAGCTGT (SEQ ID NO: 111) | att_{BTn7} | ScreenpGRG |
| | | | | integrations |
| | opir3-R | GATGACGGTTTGTCACATGGA (SEQ ID NO: 112) | pir | |
| | opir3-in | ACGTCCATCATGACCTTGAGTCTCAT | | |
| | | AAAAAAACCTCATCATTCTGTATATCTTAACGCC | | |
| | | (SEQ ID NO: 113) | | |
| TP114ΔrepA:: | oRep1- | CGTTAAAAAAGACCCGGTCACTGGTCAGAACACACTCAGA | pKD3 | Replace |
| cat- | F | GTGTAGGCTGGAGCTGCTTC (SEQ ID NO: 114) | | TP114-083 by |
| OriV_{R6K} | | | | cat-oriV_{R6K} |
| | oRep1- | AAGGGGCTGAAGAGAGTGCCGATTGTATCAGGCAGCTA | | |
| | R | AACTGTCAGACCAAGTTTACTC (SEQ ID NO: 115) | | |
| | oRep2- | GATTGTATCAGGCAGCTAAA (SEQ ID NO: 116) | TP114 | Screen |
| | F | | | TP114ΔrepA:: |
| | | | | cat-oriV_{R6K} |
| | oRep2- | CTGGTCAGAACACACTCAGA (SEQ ID NO: 117) | | |
| | R | | | |
| pNA01 | oNA5- | CTTAATTAATTAATCCAGAGGCATC | pSIM7 | OriV_{pBBR1} for |
| | F | GCCTGCCCCTCCCTTTT | | pNA01 and |
| | | GGTGTCCA (SEQ ID NO: 118) | | pNA02 |
| | oNA5- | TCTAGATTCAGCTGAATTCCCGGGTGCAC | | pNA02 |
| | R | CGAGGCGGCTACAGCCGATAGTCTGGAA | | |
| | | (SEQ ID NO: 119) | | |
| | oNA6- | CAGGCGATGCCTCTGGATTAATTAATTAAG | pREC1 | tetB for pNA01 |
| | F | GCTTGGATTCTCACCAATAA (SEQ ID NO: 120) | | and pNA02 |
| | oNA6- | TTAAAAATGAAGTTTTAAATCAATCTAAAGTAT | | |
| | R | ATATAGCCAGTTTTGGGGAAGG (SEQ ID NO: 121) | | |
| | oNA7- | GTGCACCCGGGAATTCAGCTGAATCTAGATACGTAGTACT | TP114 | OriT_{TP114} for |
| | F | AAGTCCTCAAGGTTCGTAGA (SEQ ID NO: 122) | | pNA01 and |
| | | | | pNA02 |
| | oNA7- | ATATACTTTAGATTGATTTAAAACTTCATTTTTAAT | | |
| | R | TTGTTACATTTCCTCTCTCTCTCT (SEQ ID NO: 123) | | |
| pNA02 | oNA8- | GGCTGTATGCACGGCATTTTTTTGTCCTTCTAAAACAC | TP114 | Nicking site |
| | F | ATAAGCTTTGTACACAAGCCCG | | deletion |
| | | (SEQ ID NO: 124) | | |
| | oNA8- | ACATGCCCGGAACGGGCTTGTGTACAAAGCTTATGT | | |
| | R | GTTTTAGAAGGACAAAAAAATGCC (SEQ ID NO: 125) | | |
| pKN30 and | okn01 | CATGCTGGAGTTCTTCGCCCACCCCAGCTTCAAAAGCGCTCT | pNA01 or | pNA backbone |
| pKN31 | -F | GCCTGCCCCTCCCTTTTG (SEQ ID NO: 126) | pNA02 | |
| | oKN01 | TGCGCCCTGAGTGCTTGCGGCAGCGTGAGGGGATCTT | | |
| | -R | TTGTTACATTTCCTCTCTCTCTC (SEQ ID NO: 127) | | |
| | oKN02 | TATGCCAATGTAGGAGGTAGAGAGAGAGAGGAAATGTAACA | pKD4 | aph-IIIa |
| | -F | AAAGATCCCCTCACGCTGC (SEQ ID NO: 128) | | |
| | oKN02 | CCCCGTCGAGCCGGTTGGACACCAAAAGGGAGGGGCAGGC | | |
| | -R | AGAGCGCTTTTGAAGCTGGG (SEQ ID NO: 129) | | |
| TP114ΔoriT:: | oriT1-F | CAAGCATTGTAACATGCCCGGAACGGGCTTGTGTACAAAG | pKD3 | cat |
| cat-tetB | | GTGTAGGCTGGAGCTGCTTC (SEQ ID NO: 130) | | |
| | oriT1-R | GGTGATTATGTGGGTTGTTTTGTGGGTTGTCAATGG | | |
| | | TGGGAATTAGCCATGGTCCATATG (SEQ ID NO: 131) | | |
| | oriT2-F | GCGCCGTTCGGGGTTGCAAAGGGGCGTCCCCTTTGGCA | oriT1-F + | Add homology |
| | | CAAGCATTGTAACATGCCCGGA (SEQ ID NO: 132) | oriT1-R | for oriT_{TP114} |
| | oriT2-R | TACCTTATTTAAAGCAATTTGCTCGCCGTTTGTGTG | product | |
| | | GGTGATTATGTGGGTTGTTTTGTG (SEQ ID NO: 133) | | |
| | oriT3-F | CCCAACTTAACTGAGAAAGACACC (SEQ ID NO: 134) | TP114 | Screen |
| | | | | OriT_{TP114} |
| | oriT3-R | GCTAGTTCTGTCTTGGTGTTGTTGT (SEQ ID NO: 135) | | Deletion |
| | | | | clones |
| TP11ΔpilV | opilV- | TCACCATCACGGCGACTACAAAGACGATGACGACAAGTAA | pKD3 | Delete the |
| Δshufflon:: | F1 | ATGGGAATTAGCCATGGTCC (SEQ ID NO: 171) | | shufflon |
| cat | | | | |
| | opilV- | TAAAAAATCACTCTGTAGTTGTTCTATCATAAGTGAATCC | | |
| | R1 | GTGTAGGCTGGAGCTGCTTC (SEQ ID NO: 172) | | |
| | opilV- | CAGTACAGGGGCGATACTTTCGTGCCAATCCGGTCGGTGG | | |
| | F2 | CATCACCATCACCATCACGGCGACTACA (SEQ ID NO: 173) | | |
| | opilV- | GGAGGAATATGCTGTCCTGG (SEQ ID NO: 174) | TP114 | Screen |
| | F3 | | | deletion |
| | opilV- | TCTGGTGGCAATAAAGTGAACT (SEQ ID NO: 175) | | |
| | R3 | | | |
| TP114ΔpilS:: | opilS1- | ATGTCTTCTATTAATATTTTAAATATGCGTTCTGTTTTTT | pKD3 | cat |
| cat | F | GTGTAGGCTGGAGCTGCTTC (SEQ ID NO: 136) | | |
| | opilS1- | TCAGGAATCAGTGCTGAAGGTCAGCGTATTGCTGTCAGATA | | |
| | R | TGGGAATTAGCCATGGTCC (SEQ ID NO: 137) | | |
| | opilS2- | TAACGTCCTGCAACACTAAT (SEQ ID NO: 138) | TP114 | Screen pits |
| | F | | | Deletion |
| | | | | clones |
| | opilS2- | GCTTATCCGATGCACATGAA (SEQ ID NO: 139) | | |
| | R | | | |
| TP114Δrci:: | orci1-F | CAAGAATCTCTACCTTCCCCCCTTTTTTGTCTGGAGGGGAT | pKD3 | cat |
| cat | | TGGGAATTAGCCATGGTCC(SEQ ID NO: 176) | | |
| | orci1-R | TAAAAAATCACTCTGTAGTTGTTCTATCATAAGTGAATCC | | |
| | | TGTGTAGGCTGGAGCTGCTT(SEQ ID NO: 177) | | |
| | orci2-R | GTGATATTGCATTTCGAAGCAAG(SEQ ID NO: 178) | TP114 | Screen rci |
| | | | | Deletion |
| | | | | clones |
| | orci2-R | CCAGGACAGCATATTCCTCC(SEQ ID NO: 179) | | |
| TP114ΔpilV: | opilV1- | CTCACTCGTACCGGGAATATTATTCTGAGGATTAAGAGCA | pKD3 | cat |
| cat | F | ATGGGAATTAGCCATGGTCC(SEQ ID NO: 180) | | |
| | opilV1- | TAAAAAATCACTCTGTAGTTGTTCTATCATAAGTGAATCC | | |
| | R | GTGTAGGCTGGAGCTGCTTC (SEQ ID NO: 181) | | |
| | opilV2- | CCGTTCCTTTGTGGCGGAAT(SEQ ID NO: 182) | TP114 | Screen pilV |
| | F | | | Deletion |
| | | | | clones |
| | opilV2- | CCAGGACAGCATATTCCTCC(SEQ ID NO: 183) | | |
| | R | | | |
| TP114Δshuffl | opilV3- | CAGTACAGGGGCGATACTTTCGTGCCAATCCGGTCGGTGG | TP114 | pilV1 |
| on::pilV1- | F | TCTGGTGGCAATAAAGTGAA(SEQ ID NO: 184) | | |
| cat | | | | |
| | opilV3- | GGACCATGGCTAATTCCCAT | | |
| | R | TTAACCGAAGGGGCAACAAT(SEQ ID NO: 185) | | |
| | opilV4- | ATTGTTGCCCCTTCGGTTAA | pKD3 | cat |
| | F | ATGGGAATTAGCCATGGTCC(SEQ ID NO: 186) | | |
| | opilV1- | TAAAAAATCACTCTGTAGTTGTTCTATCATAAGTGAATCC | | |
| | R | GTGTAGGCTGGAGCTGCTTC (SEQ ID NO: 187) | | |
| TP114Δshuffl | opilV5- | CAGTACAGGGGCGATACTTTCGTGCCAATCCGGTATCTGG | TP114 | pilV2 |
| on::pilV2-cat | F | ACAACGGCAAAAGTGAACTT(SEQ ID NO: 188) | | |
| | opilV5- | GGACCATGGCTAATTCCCAT | | |
| | R | TTAAATCCCAGCACCAGGAA(SEQ ID NO: 189) | | |
| | opilV6- | TTCCTGGTGCTGGGATTTAAATGGGAATTAGCCATGGTCC | pKD3 | cat |
| | F | (SEQ ID NO: 190) | | |
| | opilV1- | TAAAAAATCACTCTGTAGTTGTTCTATCATAAGTGAATCC | | |
| | R | GTGTAGGCTGGAGCTGCTTC (SEQ ID NO: 191) | | |
| TP114Δshuffl | opilV7- | CAGTACAGGGGCGATACTTTCGTGCCAATCCGGTACGTGG | TP114 | pilV3 |
| on::pilV3-cat | F | AAAAAAATTGGCGCAGGTGA(SEQ ID NO: 192) | | |
| | opilV7- | GGACCATGGCTAATTCCCA | | |
| | R | TTCACTGGCAAATGGCGTAAA(SEQ ID NO: 193) | | |
| | opilV8- | TTTACGCCATTTGCCAGTGA | pKD3 | cat |
| | F | ATGGGAATTAGCCATGGTCC(SEQ ID NO: 194) | | |
| | opilV1- | TAAAAAATCACTCTGTAGTTGTTCTATCATAAGTGAATCC | | |
| | R | GTGTAGGCTGGAGCTGCTTC (SEQ ID NO: 195) | | |
| TP114Δshuffl | opilV9- | CAGTACAGGGGCGATACTTTCGTGCCAATCCGGTACGTGG | TP114 | pilV3′ |
| on::pilV3′- | F | AGAAGGGCATCAGGTAGCAC(SEQ ID NO: 196) | | |
| cat | | | | |
| | opilV9- | GGACCATGGCTAATTCCCAT | | |
| | R | TCACTGGCAAACCACGATGT(SEQ ID NO: 197) | | |
| | opilV10 | ACATCGTGGTTTGCCAGTGA | pKD3 | cat |
| | -F | ATGGGAATTAGCCATGGTCC(SEQ ID NO: 198) | | |
| | opilV1- | TAAAAAATCACTCTGTAGTTGTTCTATCATAAGTGAATCC | | |
| | R | GTGTAGGCTGGAGCTGCTTC (SEQ ID NO: 199) | | |
| TP114Δshuffl | opilV11 | CAGTACAGGGGCGATACTTTCGTGCCAATCCGGTGTGTGG | TP114 | pilV4 |
| on::pilV4- | -F | AGGGCATTAGGTGGAAAGCT(SEQ ID NO: 200) | | |
| cat | | | | |
| | opilV11 | GGACCATGGCTAATTCCCAT | | |
| | -R | TTAATTGAGAGTTACACAGG(SEQ ID NO: 201) | | |
| | opilV12 | CCTGTGTAACTCTCAATTAA | pKD3 | cat |
| | -F | ATGGGAATTAGCCATGGTCC(SEQ ID NO: 202) | | |
| | opilVI- | TAAAAAATCACTCTGTAGTTGTTCTATCATAAGTGAATCC | | |
| | R | GTGTAGGCTGGAGCTGCTTC (SEQ ID NO: 203) | | |
| TP114Δshuffl | opilV13 | CAGTACAGGGGCGATACTTTCGTGCCAATCCGGTGTGTGG | TP114 | pilV4′ |
| on::pilV4′- | -F | AGAACTTCCGGTTCCTCTAA(SEQ ID NO: 204) | | |
| cat | | | | |
| | opilV13 | GGACCATGGCTAATTCCCATTTAAGT | | |
| | -R | TTGGTATCCAAAAA(SEQ ID NO: 205) | | |
| | opilV14 | TTTTTGGATACCAAACTTAAATGGGA | pKD3 | cat |
| | -F | ATTAGCCATGGTCC(SEQ ID NO: 206) | | |
| | opilV1- | TAAAAAATCACTCTGTAGTTGTTCTATCATAAGTGAATCC | | |
| | R | GTGTAGGCTGGAGCTGCTTC (SEQ ID NO: 207) | | |
| TP114Δshuffl | opilV15 | CAGTACAGGGGCGATACTTTCGTGCCAATCCGGTACGTGG | TP114 | pilV5 |
| on::pilV5- | -F | CAGAAAAATGGCGGCGGTAC(SEQ ID NO: 208) | | |
| cat | | | | |
| | opilV15 | GGACCATGGCTAATTCCCAT | | |
| | -R | TCACTGACACAATGCATAAG(SEQ ID NO: 209) | | |
| | opilV16 | CTTATGCATTGTGTCAGTGA | pKD3 | cat |
| | -F | ATGGGAATTAGCCATGGTCC(SEQ ID NO: 210) | | |
| | opilVI- | TAAAAAATCACTCTGTAGTTGTTCTATCATAAGTGAATCC | | |
| | R | GTGTAGGCTGGAGCTGCTTC (SEQ ID NO: 211) | | |
| TP114Δshuffl | opilV17 | CAGTACAGGGGCGATACTTTCGTGCCAATCCGGTTCGTGG | TP114 | pilV5′ |
| on::pilV5′- | -F | AAATCAATAGGTTCATGTGC(SEQ ID NO: 212) | | |
| cat | | | | |
| | opilV17 | GGACCATGGCTAATTCCCATT | | |
| | -R | TAGCGGAAGCAGTGAACAG(SEQ ID NO: 213) | | |
| | opilV18 | CTGTTCACTGCTTCCGCTAA | pKD3 | cat |
| | -F | ATGGGAATTAGCCATGGTCC(SEQ ID NO: 214) | | |
| | opilV1- | TAAAAAATCACTCTGTAGTTGTTCTATCATAAGTGAATCC | | |
| | R | GTGTAGGCTGGAGCTGCTTC (SEQ ID NO: 215) | | |
| pPilS | opPilS- | CTGTCAGACCAAGTTTACTC(SEQ ID NO: 216) | pBAD30 | oriV_{p15A}, araC |
| | F1 | | | P_{BAD} |
| | opPIIS- | TTTTGCCTCCTA | | |
| | R1 | CGCTAGCCCAAAAAAACGGG(SEQ ID NO: 217) | | |
| | opPilS- | AACCGTATTACCGCCTTTGAG (SEQ ID NO: 218) | pSB1C3 | cat |
| | F2 | | | |
| | opPilS- | TCAATCTAAAGTATATATGAGTAAACTTGGTCTGACAG | | |
| | R2 | GTAAACTTGGTCTGACAGCTCG(SEQ ID NO: 219) | | |
| | opPilS- | TCTCCATACCCGTTTTTTTGGGCTAGCGTAGGAGGCAAAA | TP114 | pilS |
| | F3 | ATGTCTTCTATTAATATTTT(SEQ ID NO: 220) | | |
| | opPilS- | GGCGAGCGGTATCAGCTCACTCAAAGGCGGTAATACGGTT | | |
| | R3 | TCAGGAATCAGTGCTGAAGG(SEQ ID NO: 221) | | |
| pilV4′ | opPilV | TCTCCATACCCGTTTTTTTGGGCTAGCGTAGGAGGCAAAA | TP114 | pilV4′ |
| | 4′-F | ATGAAAAAGACAGATAAAGG(SEQ ID NO: 222) | | |
| | opPilV | GGCGAGCGGTATCAGCTCACTCAAAGGCGGTAATACGGTTT | | |
| | 4′-R | TAAGTTTGGTATCCAAAAA (SEQ ID NO: 223) | | |

^{a}Oligonucleotide's priming site are underlined.
^{b}Mutation introduced in the oligonucleotides are in bold.

DNA Purification.

Purification of DNA was performed between each step of plasmid assembly to avoid buffer incompatibility or stop enzymatic reactions. PCR reactions were generally purified by Solid Phase Reversible Immobilization (SPRI) using Agencourt Ampure XP DNA binding beads (Beckman Coulter) according to the manufacturer's guidelines. When DNA samples were digested with restriction enzymes, DNA was purified using DNA Clean and Concentrator (ZYMO RESEARCH) following manufacturer's recommendation for cell suspension DNA purification protocol. After purification, DNA concentration and purity was routinely assessed using a Nanodrop spectrophotometer when necessary.

DNA transformation into E. coli by electroporation. Routine plasmid transformations were performed by electroporation. Electrocompetent E. coli strains were prepared from 20 mL of LB broth. Cultures reaching exponential growth phase of 0.6 optical density at 600 nanometers (OD₆₀₀ₙₘ) were then washed three times in sterile distilled water. Cells were then resuspended in 200 μL of water and distributed in 40 μL aliquots. The DNA was then added to the electrocompetent cells and the mixture was transferred in a 1 mm electroporation cuvette. Cells were electroporated using a pulse of 1.8 kV, 25 μF and 200Ω for 5 ms. Cells were then resuspended in 1 mL of non-selective LB medium and recovered for 1 hour before plating on selective media.

DNA transformation into E. coli by heat-shock. Heat-shock transformation was mostly used to clone Gibson assembly products. Chemically competent cells were prepared according to the rubidium chloride protocol as described previously (Green et al., 2013). Chemically competent cells were flash-frozen and conserved at −80° C. before use. Gibson assembly products were directly transformed into EC100Dpir+ chemically competent cells at a 1/10 volume ratio. Routinely, up to 10 μL of DNA was added to 100 μL competent cells before transformation by a 45 seconds heat shock at 42° C. Cells were then resuspended in 1 mL of non-selective LB medium and let to recover for 1 hour at 37° C. before plating on selective media.

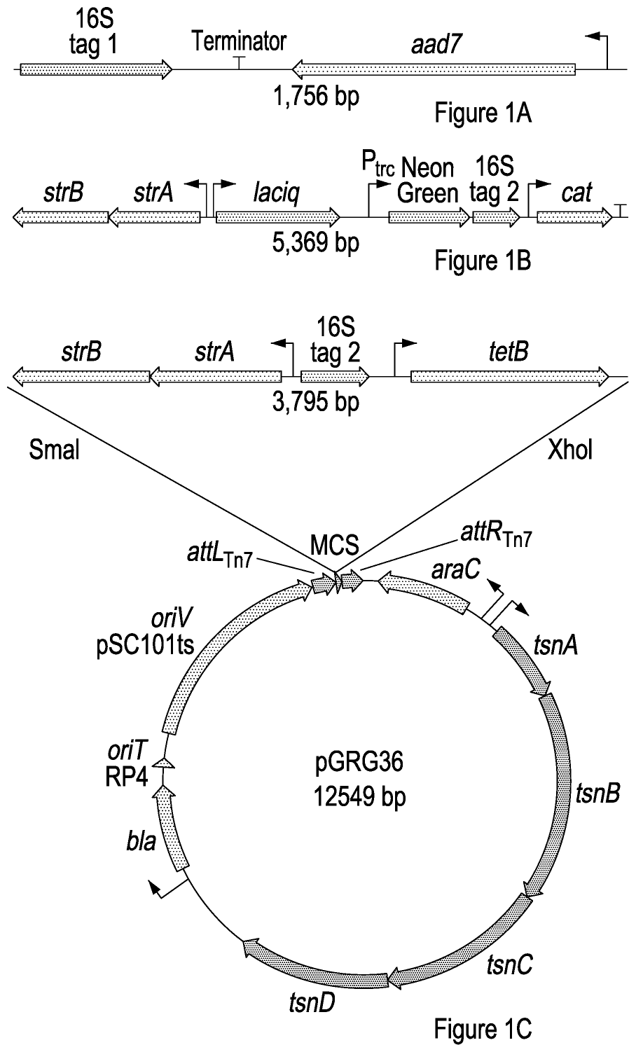

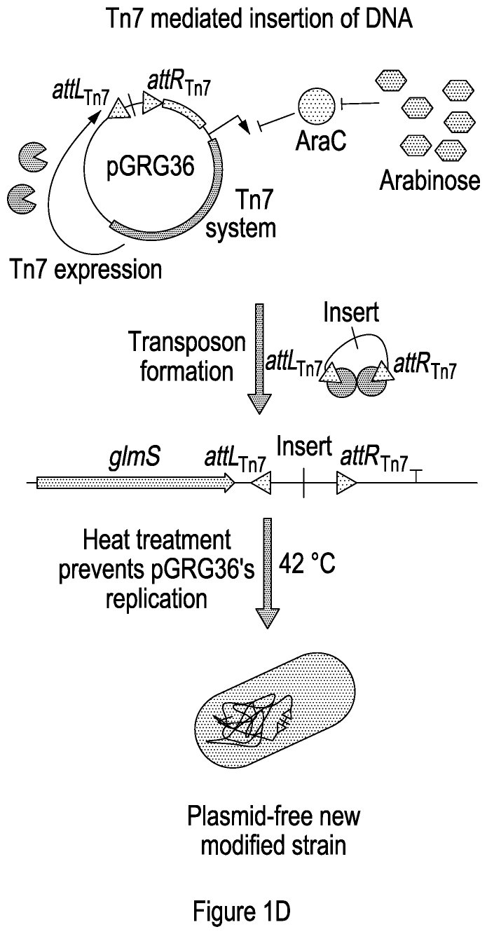

Introduction of selection markers in Escherichia coli Nissle 1917 (EcN) strains for conjugation quantification. The modified EcN strains were obtained by Tn7 insertion of the antibiotic resistance cassettes as described previously (McKenzie et al., 2006). Integration was verified by PCR using corresponding primers as described in table 2. Loss of ampicillin resistance was confirmed to verify plasmid elimination. More specifically, the pGRG36 vector was purified from E. coli EC100Dpir+ and digested with SmaI+XhoI. The inserts were amplified by PCR using their corresponding primers (Table 2) and inserted by Gibson assembly between attL_{Tn7} and attR_{Tn7} sites of the digested pGRG36 plasmid (FIG. 1). The Gibson assembly products were then transformed in chemically competent E. coli EC100Dpir+ strain. The resulting plasmids were analyzed using restriction enzymes, and positive clones were transformed into E. coli MFDpir+ (Ferrières et al., 2010). Plasmids were mobilized from E. coli MFDpir+ to EcN by conjugation. To mediate cassette insertion into the terminator of glmS, EcN was first cultivated at 30° C. in LB with arabinose until 0.6 OD₆₀₀ₙₘ. Cells were next heat-shocked at 42° C. for 1 hour and incubated at 37° C. overnight to allow for plasmid clearance. An aliquot of the bacterial culture was then streaked onto a LB agar plate. 20 colonies were analyzed, and colonies that only grew in the absence of ampicillin, but contained the insert's selection markers were then investigated by PCR using the appropriate primers listed in table 2.

Construction of E. coli KN01ΔdapA. A DAP auxotrophic variant was also obtained through the deletion of the dapA gene in EcN (Born et al., 1999) by recombineering using pSIM6. DAP auxotrophy was shown to be a good marker to discriminate donor and recipient strains for conjugation without hindering transfer frequencies as DAP auxotroph reversion was never reported (Ronchel et al, 2001) and, when complemented, DAP auxotrophy has little impact on the fitness of the bacterium (Allard et al., 2015). To generate a DAP auxotrophic strain, the aph-IIIa resistance cassette of pKD4 was amplified by PCR with added homology for the regions flanking dapA. A second PCR round on the purified PCR product then allowed to increase the length of homology. Recombineering was performed in EcN using pSIM6 as described previously (Datta et al., 2006). Briefly, EcN containing pSIM6 was electroporated with the purified PCR product. Kanamycin resistant bacteria were selected and DAP auxotrophy confirmed. Insertion of the cassette and deletion of dapA were also verified by PCR with corresponding primers (Table 2). After the confirmation of the cassette insertion in the dapA gene locus, the strain was cured from pSIM6 by heatshock at 42° C. for 1 hour followed by overnight incubation at 37° C. The culture was then streaked on selective plates to identify Ap sensitive clones, which were next transformed with pCP20 to eliminate the resistance cassette as described previously (Datsenko et al., 2000). pCP20 plasmid was cured by heat-shock following the same procedure as before. Next, the SmSp insert was added in the genome of EcNΔdapA strain to complete KN01ΔdapA.

In vitro conjugation assay. For all in vitro conjugation assays, donor strains were KN01ΔdapA and recipient strains were KN02 unless specified otherwise. The strains were grown from frozen stocks 18 hours prior to conjugation experiments, mixed at a 1:1 volume ratio (100 μL each), centrifuged at maximum speed for one minute and washed in 200 μL of LB without antibiotics. The bacteria mix was then spun down and, either resuspended in 5 μL of LB broth and deposited on a LB agar plate with DAP, or resuspended to 1.0 OD₆₀₀ₙₘ in LB broth with DAP. The cell mix was then incubated at 37° C. for the desired conjugation time before being resuspended in 800 μL sterile PBS and diluted 1/10 serially in sterile PBS to avoid growth during dilution and plating. 5 μL of each dilution were then spotted in duplicates on LB plates with appropriate antibiotics to select donors, recipients and transconjugants, and the number of Colony Forming Units (CFU) was counted. All conjugation frequencies were calculated by dividing the number of transconjugants by the total number of recipient CFUs. The conjugation frequencies per donor were however equivalent (data not shown) since cells were always mixed 1:1. All conjugation experiments were repeated with at least three independent biological replicates.

Mouse model. All mice-related protocols were designed in compliance with our institution Animal Care Comity Guidelines and were strictly evaluated to avoid animal suffering. Animals were provided with water and regular chow ad libitum throughout all experiments. Animals were housed in individually ventilated cages and no more than 5 individuals shared the same cage. All animals used were C57 BL/6 females of 16-20 g (Charles River) and were given a 3 days adaptation period upon arrival. Animal weight and health was monitored daily throughout each experiment. No significant health or weight loss was noted for any mouse in any experiment. For Sm treated mice groups, Sm working concentration was first evaluated to maximize Enterobacteriaceae clearance and EcN colonization (Kotula et al., 2014). A concentration of 1 g/L of Sm was chosen and added to drinking water 2 days prior to gavages for all Sm-treated mice groups. From that point, water bottles were refreshed every 3 days to maintain optimal Sm activity. Bacterial load was monitored in feces sampling at specified time points. At the end of the experiment, animals were anesthetized with isoflurane and sacrificed by cervical dislocation. Animals were then dissected to reveal the colonization pattern and gut bacterial content was evaluated by CFU.

Mice inoculum preparation. Two days prior to gavages, the appropriate strains were streaked from frozen stocks onto MacConkey selective plates and incubated overnight at 37° C. The next day, colonies were inoculated in selective LB broth at 37° C. Three to four hours prior to mice oral challenge, the strains were subcultured again with a large inoculum (200 μL or 500 μL) in 20 mL selective LB broth and incubated at 37° C. until 0.6±0.1 OD₆₀₀ₙₘ was reached. The cells were then washed once in PBS and concentrated in a volume equivalent to 6.0 OD₆₀₀ₙₘ. An aliquot of the inoculum was used to evaluate cell concentration. 100 μL of the final cell suspension were administered orally to each mouse (approximately 1×10⁸ CFU).

Feces and tissues processing. Collection tubes were prepared prior to the experiment by adding 500 μL of PBS and a single 0.2 mm glass bead to a sterile 1.5 mL microtube. Then, tubes were weighted before and after sampling to normalize CFU by sample weight. Samples were homogenized in a FastPrep-24 (MP) bead beater for 1 minute at maximum speed. Then, the homogenates were centrifuged at 500×g for 30 second to avoid possible pipetting of larger debris. Centrifugation has shown no significant impact on retrieved CFU (data not shown). The samples were then serially diluted 1/10 in sterile PBS from 10⁰ to 10⁻⁷ of the initial concentration and 2.5 μL of each dilution was spotted on selective MacConkey plates in technical duplicates. CFUs/mg sample were calculated as a function of the sample measured weight. For each experiment, total Enterobacteriaceae clearance was also followed on MacConkey plates without antibiotics as a control for Sm treatment (data not shown).

Mice dissection and EcN colonization pattern assessment. The mice were sacrificed on day 4 and dissected to extract the duodenum, jejunum, ileum, caecum, ascending colon and descending colon. To distinguish the parts of the intestine, the first 3 centimeters (cm) of small intestine attached to the stomach were considered to be the duodenum, the 6 central cm the jejunum, and the last 6 cm (closest to the caecum) the ileum. The ascending and descending colons were the exact halves of the colon. Two spaced quarters of each section were sampled for CFUs analysis. The longitudinal half of the caecum was used for CFU as well. Since the caecum is a large and distinct structure of the mouse intestine, and since EcN colonizes strongly the caecum, this region was chosen as a representative part of the intestine to study colonization and conjugative bacterial cell treatments.

In vivo conjugation mouse model. For in vivo conjugation experiment, mice were orally challenged with the recipient strain 2 or 12 hours prior to the introduction of donor strain. This, in order to avoid possible plasmid transfer in the PBS solution prior to gavage. Conjugation was then monitored by feces sampling at specified time points. Mice were sacrificed at the end of the experiment and caecum was extracted to verify conjugation levels in the murine gut. Feces were homogenized and CFU were acquired on MacConkey plates as described in the Feces and tissue processing section.

Statistical analysis. Statistical significance was performed on the logarithmic value of the data using One-way ANOVA unless specified otherwise. P-values are directly indicated on the graphs and represent statistical significance of the difference between the two data groups. Differences in the data were considered significant when the P-value was bellow 0.05.

1.1 Escherichia coli Nissle 1917 Modified Strain Construction

Generation of antibiotic-resistant EcN variants. Since EcN had no natural antibiotic resistance phenotype (Sonnenborn et al., 2009), efficiency of conjugation between two strains of EcN was impossible to quantify. The use of two different resistance markers was essential to distinguish between the donor and recipient strains. Furthermore, the presence of an antibiotic resistance marker on a conjugative plasmid allowed for the distinction between recipients and transconjugants. Several strains of EcN were therefore developed to allow quantification of conjugation efficiency. One way to generate an antibiotic resistant variant of a strain was to insert a resistance gene in its chromosome. Integration of DNA in the chromosome of a bacterium was efficiently achieved using a Tn7-based system (McKenzie, 2006). This system used a plasmid, pGRG36, as a vector for the expression of the Tn7 machinery, but also as a backbone for the insertion of a DNA sequence of interest. The DNA sequence of interest required cloning between the attL_{Tn7} and attR_{Tn7} sites of pGRG36 so that it could be inserted in the terminator sequence of glmS. The Tn7 strategy was used to insert antibiotic resistance cassettes into EcN and created three different strains (FIG. 1). Those strains were all resistant to Sm which was used to hinder the microbiota in vivo. Additional resistance phenotypes were unique for each three strains. For instance, the donor KN01 was also resistant to Sp, the recipients KN02 was also resistant to Cm, and KN03 was also resistant to Tc.

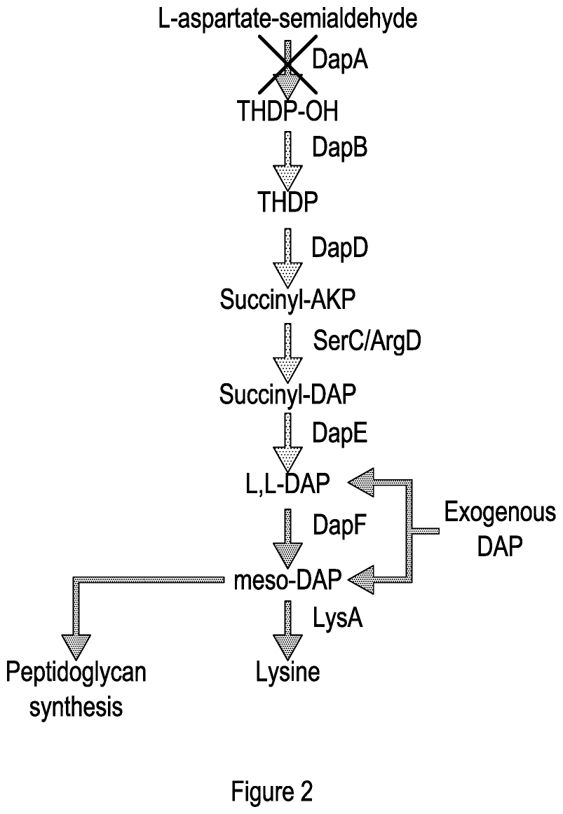

Auxotrophy as a selection marker for conjugation. As opposed to antibiotic resistance which allows a cell to grow in the presence of an antibiotic, auxotrophy prevents a cell from growing under normal conditions. This can be particularly useful to further distinguish donor and recipient strains in a conjugation experiment as no known reversion mechanisms were yet reported and auxotrophic donor strains present no defect in their ability to conjugate. EcN was first transformed with pSIM6, a plasmid that expressed the lambda red recombination system. Then, the dapA gene was replaced with an antibiotic resistance cassette as previously described (Datsenko et al., 2000). The deletion of dapA interrupted the lysine biosynthesis pathway as well as the peptidoglycan wall synthesis (FIG. 2). The cell therefore became unable to synthesize its cell wall and the lysine amino acid. Both functions are essential for cell survival under normal conditions. However, the mutation could be complemented by an exogenous source of DAP (Allard et al., 2015). Plasmid pGRG36-SmSp was then used to insert SmSp resistance gene in EcNΔdapA's chromosome thereby creating KN01ΔdapA. This donor strain was not able to grow without DAP and allowed for a better distinction of transconjugants and recipients. Also, as a control, it was verified that the deletion of dapA did not affect the conjugation efficiency, which it did not (data not shown).

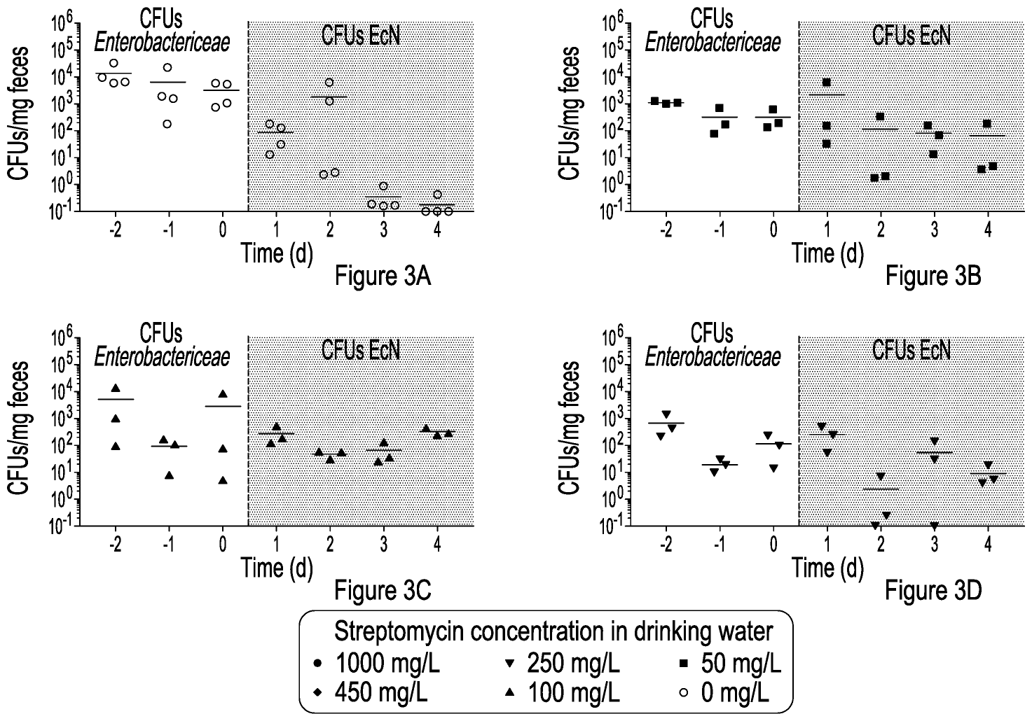

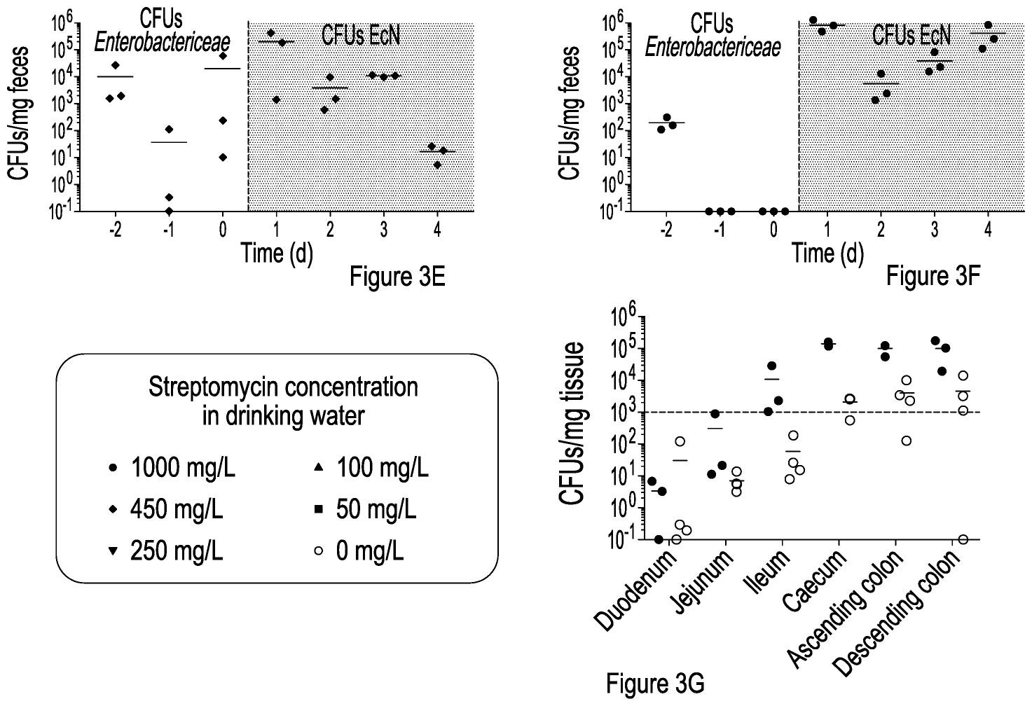

EcN colonized the murine gut. In order to compare conjugation efficiencies of several conjugative plasmids in vitro and in vivo, the ability of EcN to colonize the murine gut was verified. Sm was previously shown to increase colonization stability of E. coli in mice, and since KN01 had the lowest Minimal Inhibitory Concentration (MIC) for Sm (Table 3), it was used in colonization assays to determine the concentration of Sm needed to (1) clear the Enterobacteriaceae from the microbiome and (2) facilitate colonization of the donor and recipient strains. Concentrations of 1,000 mg/L (FIG. 3.A), 400 mg/L (FIG. 3.B), 250 mg/L (FIG. 3.C), 100 mg/L (FIG. 3.D), 50 mg/L (FIG. 3.E) and 0 g/L (FIG. 3.F) of Sm were tested. 1 g/L of Sm in drinking water cleared Enterobactericeae while allowing stable colonization of the KN01 strain. This concentration was used in subsequent experiments. Colonization pattern of EcN was also addressed both in Sm treated and untreated mice by analyzing KN01's CFU density in the duodenum, jejunum, ileum, caecum, ascending colon and descending colon (FIG. 3.G). Colonization was higher in all part on the intestine of Sm treated mice and was particularly high (>10³ CFU/mg tissue) for parts of the intestine between the ileum and the anus. EcN was therefore a good strain for the quantification of conjugation in vivo because of its ability to colonize different regions of the intestinal tract. However, the caecum was used for subsequent conjugation quantification, as it is a distinct structure that yielded higher density of KN01 (10⁴ CFU/mg tissue).

TABLE 3
Minimal inhibitory concentration (MIC) of EcN strains.

| Strain | Sm | | Sp | | Ap | Cm | |
|---|---|---|---|---|---|---|---|
| EcN | 25 | mg/L | 50 | mg/L | <25 mg/L | <8 | mg/L |
| KN01 | 800 | mg/L | 3 | g/L | <25 mg/L | <8 | mg/L |
| KN02 | >1.6 | g/L | 50 | mg/L | <25 mg/L | 264 | mg/L |

1.2—Comparison of Conjugative Transfer Efficiency In Vitro Versus In Vivo

Selection of bacterial conjugative plasmids. To find the most efficient bacterial conjugative system for the transfer of DNA in vivo, six conjugative plasmids were chosen. Those six plasmids span six different incompatibility families (Table 4). Incompatibility families are a classification based on the ability of two plasmids to co-exist at the same time in a cell. For two plasmids to belong to the same incompatibility family, they have to be unable to be maintained simultaneously in a cell. There are two major ways plasmids can be incompatible (1) by inhibiting the transfer of the other plasmid inside the hosting cell and (2) by strong similarity between their maintenance modules. By selecting plasmids from different incompatibility families, plasmids had a higher chance of being more phylogenetically distant from one another. The plasmids were also selected for their reported in vitro transfer efficiencies (Bradley et al., 1980).

TABLE 4
Conjugative plasmid selected for this example.

| | | Size | | | Accession | |
|---|---|---|---|---|---|---|
| Name | Incompatibility | (bp) | CDS | Resistances* | number | Supplier/Source |
| pOX38 | IncF1 | 59,705 | 62 | Sp^{R}, Tc^{R}, Su^{R} | MF370216.1** | Laura Frost's |
| | | | | | | research group |
| R6K | IncX2 | 39,872 | 52 | Ap^{R}, Sm^{R} | None*** | DSM-4245 |
| | | | | | | (DSMZ) |
| TP114 | Incl2 | 64,818 | 92 | Km^{R} | MF521836.1 | DSM-4246 |
| | | | | | | (DSMZ) |
| pVCR94ΔX3 | IncC | 121,195 | 152 | Km^{R} | KF551948.1** | Carraro et al. |
| | | | | | | 2017 |
| R388 | IncW | 33,913 | 48 | Su^{R}, Tm^{R} | NC_028464 | DSM-5189 |
| | | | | | | (DSMZ) |
| RP4 | IncP1α | 60096 | 70 | Ap^{R}, Tc^{R} | BN000925.1 | DSM-3876 |
| | | | | | | (DSMZ) |

*As experimentally tested in 96 well plates
**NCBI available sequence are engineered variant resistant to other antibiotics
***Available at http://www.sanger.ac.uk/resources/downloads/plasmids/

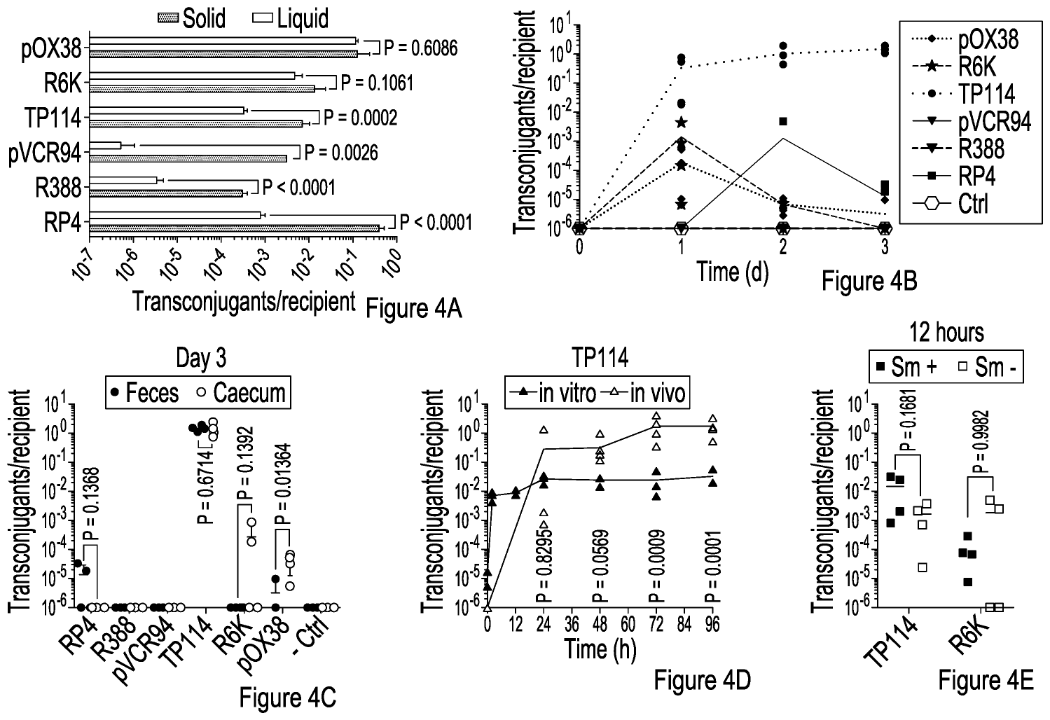

Bacterial conjugation efficiency was affected by the physical properties of the environment. The six conjugative plasmids were transferred into the KN01ΔdapA and KN01 strains, which constituted the donor strains for the following experiments. Conjugation experiments, between KN01ΔdapA containing one of the conjugative plasmids and KN02 as the recipient, were carried both on agar plates (solid mating) and in broth (liquid mating) in an effort to predict the conjugation efficiency of conjugative plasmids in vivo (FIG. 4.A). Conjugation on solid support allows conjugative plasmids to transfer without the need for mating pair stabilization as cells are immobilized on a solid surface and no shearing forces are present (Bradley, 1984). However, conjugation in liquid is a much more instable environment as cells must firmly grip to each other to avoid conjugation interruption by shearing forces as cells are constantly moving in the liquid. Most plasmids transferred efficiently on agar plates, but pOX38 and R6K were able to conjugate at similar frequencies in both agar and broth. Conjugation mediated by TP114 seemed only mildly affected under liquid transfer conditions.

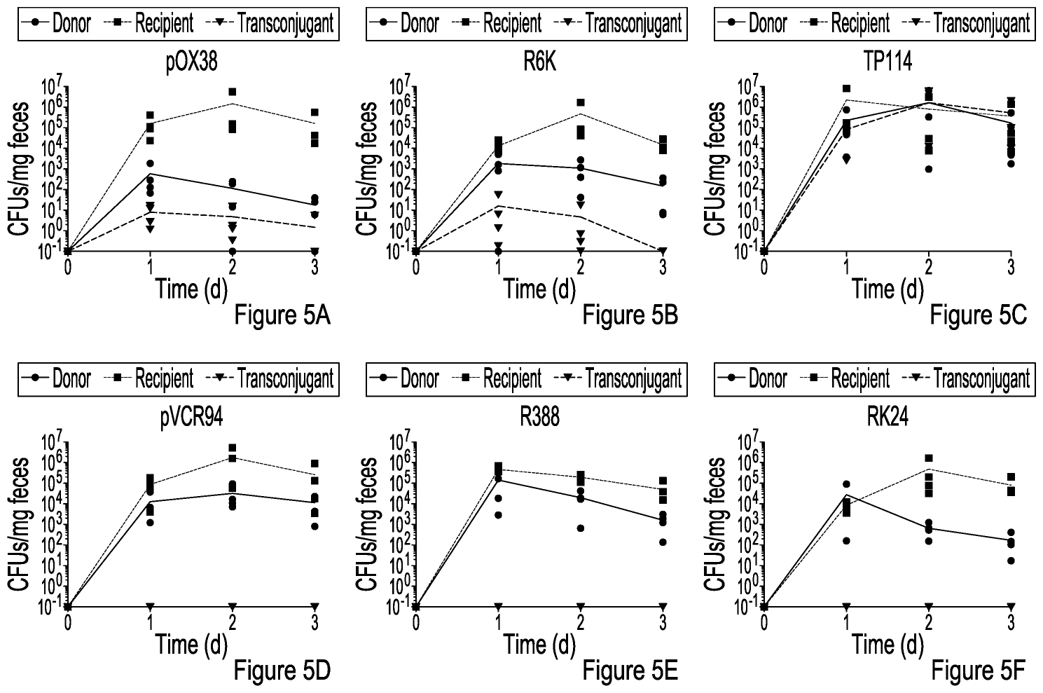

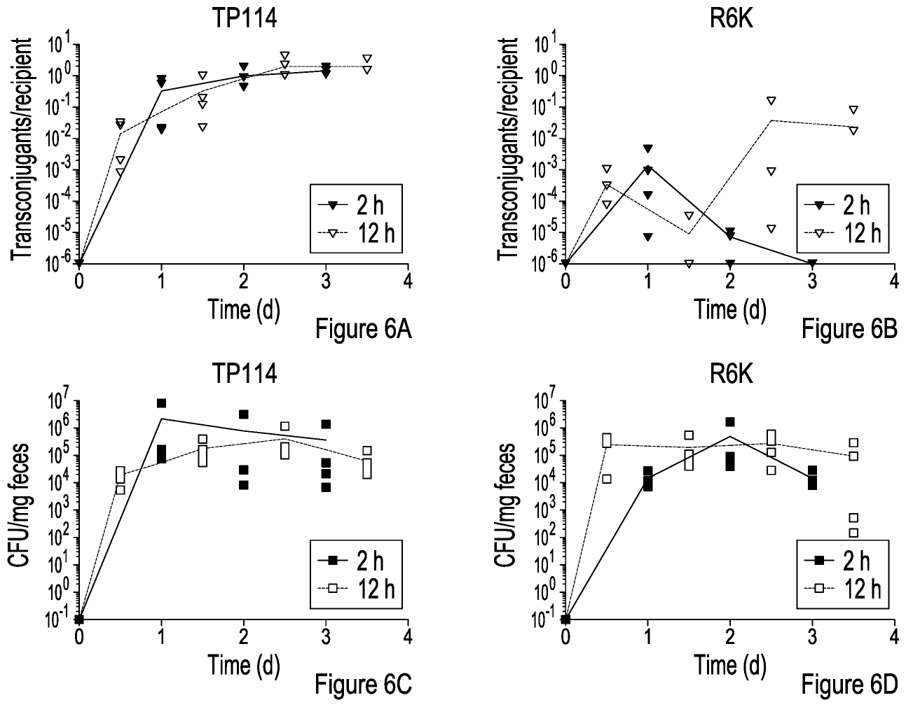

Discrepancies of conjugation efficiency between in vivo conditions and in vitro laboratory condition. The conjugative plasmids which could be of interest for therapeutic applications (in vivo) and could constitute the most appropriate transfer machinery for the bacterial conjugative bacterial cell system was determined. Conjugation between KN01 and KN02, used as donor and recipient, respectively, was performed in a Sm-treated conjugation mouse model. The mice were fed with the recipient strain 2 hours prior to the introduction of the donor strain. The proportion of transconjugants was monitored for three days in feces (FIG. 4.B). On the third day of the experiment, mice were sacrificed and the proportion of transconjugants was addressed in the caecum. The conjugation results in the caecum were consistent with those found in the feces (FIG. 4.C). Raw CFUs data for donors, recipients, and transconjugants are shown for each plasmid in FIG. 5. Only three plasmids (pOX38, R6K and TP114) had been able to conjugate reproducibly in mice. Among those, TP114 had a transfer rate nearly 100-fold higher in vivo than in vitro (FIG. 4.A). Also, TP114 was able to transfer between ˜240 and >1,400,000-fold more efficiently than all other tested plasmids at any of the tested timepoint. In vivo transfer activity of TP114 was also confirmed in an additional group of 5 mice and was also compared to the one measured in vitro on solid medium using the same time course (FIG. 4.D). Conjugation of TP114 over 48 hours or more yielded very little improvement in transfer rates in vitro compared to in vivo conditions. To verify if conjugation in Sm-treated mice reflects the transfer rates in an undisturbed microbiome, 12-hour conjugation experiments were carried in both Sm-treated or untreated mouse model with TP114 and R6K (FIG. 4.E). Transfer rates were similar in both conditions, hereby showing that the presence of the intestinal microbiome did not affect conjugation by TP114 or R6K. The influence of colonization time between recipient and donor introduction was also investigated with 2 or 12 hours between gavages. This parameter had little influence on the overall conjugation frequencies of TP114 (FIG. 6.A) and R6K (FIG. 6.B). In addition, colonization levels of recipient strains for both TP114 and R6K were similar regardless of the period between gavages (FIGS. 6.C and 6.D). However, TP114 was the only conjugative plasmids tested capable of transferring in vivo with an efficiency rate of nearly 100%, it was therefore considered the most interesting candidate to use as the transfer machinery of the COP.

### EXAMPLE II—IDENTIFICATION OF GENES AND GENETIC ELEMENTS REQUIRED FOR IN VIVO CONJUGATIVE DELIVERY OF DNA BY TP114

Strains, plasmids and growth conditions. All strains and plasmids are described in Table 1. All plasmid sequences are provided in the sequence appendix. Oligonucleotides used in this example, strain growth conditions, DNA manipulation, plasmid construction, recombineering and routine transformation can be found in the Material and Method section of the Example I.

Sequencing of TP114. TP114 was acquired from DSMZ (DSM-4246) and transferred from E. coli K12 J53-2 by conjugation into E. coli MG1655Nx^{R}. The resulting strain was grown at 37° C. in selective LB broth to obtain sufficient DNA for sequencing. An Illumina library was prepared using the QIAseq FX Library kit (Qiagen) from size-selected genomic DNA fragments of approximately 400 to 600 bp. The Illumina library was sequenced on a MiSeq instrument using paired-end reads of 300 bp to assemble longer composite reads covering the entire insert (Rodrigue et al., 2010). A MinION (Oxford Nanopore Technologies, UK) sequencing library was also prepared using 1.5 μg of high-molecular weight genomic DNA and the R9 Nanopore sequencing kit (SQK-NSK007, Oxford Nanopore Technologies, UK). Illumina sequencing reads were assembled with the Roche gsAssembler version 2.6 either de novo or using reference sequences from other conjugative plasmids from the IncI2 family (R721, AP002527.1; pChi7122, FR851304; pRM12761, CP007134.1; pSLy21, NZ_CP016405.1). Large de novo and reference contigs were then manually assembled and scaffolded with high-quality MinION reads using BLASTn. Finally, 10 regions of 1.5 kb selected based on lower read coverage were re-sequenced by Sanger sequencing to confirm the assembly with corresponding primers (Table 2). The resulting circular sequence of 64,818 bp (43% G+C content) was submitted to the RAST annotation server (Aziz et al., 2008), and a total of 92 open reading frames (ORF) were predicted. The annotation was then adjusted to name homologous genes consistently between TP114 and the reference IncI2 plasmid R721 (GenBank: AP002527.1).

Analysis of TP114 gene function. In silico analysis of TP114 gene function was performed using both CDsearch (Marchler-Bauer et al., 2017) and BLASTp (Altschul et al., 1990). A protein multi-fasta file was first generated for all 92 Open Reading Frames (ORF) predicted by RAST (Aziz et al., 2008). The multi-fasta file was processed by CDsearch to find conserved protein domains and attribute protein families, or superfamilies, to each protein coding genes of TP114. The multi-fasta file was also submitted to BLAST to identify putative protein homologues when CDsearch would fail to identify any protein domain with high confidence (e-value <1×10⁻¹⁵). Both analyses were performed using default parameters. BLAST hits with high identity levels were used to attribute putative functions only when more than five hits showed the same result. Proteins that failed at matching these criteria were considered of unknown function.

Comparative genomics. Gene content comparison was performed on TP114 against a database of 7 randomly selected plasmids of the IncI1 and IncI2 subfamilies based only on sequence availability (Table 5). The BRIG stand-alone software (Alikhan et al., 2011) was used to perform BLAST based homology analysis between TP114 and each plasmid group. Homology was analysed using both nucleotide sequence of the whole plasmids and amino-acid sequence of the coding genes. Conservation of genes was evaluated using the sequence identity cut-offs of 100%, 70%, and 50%. The identity percentage was calculated by attributing scores of −2 for mismatches, +1 for matches and a linear cost for insertion/deletion. Genes were then categorized as core genes when present in 100% of the plasmids, soft core genes when present in above 50% of the plasmids, or accessory genes when present in less than 50% of the plasmids.

Deletion of pilS in TP114. An FRT flanked cat gene was amplified from pKD3 was used to delete pilS in TP114 by recombineering (Datsenko et al., 2000). The recombinant clones of MG1655Rf^{R} were then screened using appropriate primers (Table 2). The pilS deletion generated TP114ΔpilS::cat, which was then transferred to E. coli strain KN01. The ability of wild type and its pilS mutant to transfer from E. coli KN01 to E. coli KN03 was assayed under solid, liquid (static), liquid with agitation and in vivo conditions.

Deletion of the pilV adhesin, the pilVC-terminus shufflon and locking of C-terminus variants of pilV. A cassette containing the cat chloramphenicol resistance gene flanked by FRT sequences was amplified from pKD3 using appropriate primers (Table 2) providing homology to the regions adjacent to the shufflase gene rci. The cassette was then inserted in TP114 by recombineering using pSIM6 in MG1655Rf^{R} to generate TP114Δrci::cat. pSIM6 was then cured by incubation of the strain at a non-permissive temperature, and next transformed with pE-FLP. This resulted in the excision of the cat gene from TP114Arci::cat, creating TP114Arci, a variant of TP114 lacking the recombination capabilities provided by rci. Then, a cassette containing a FRT flanked cat gene with homology to regions adjacent to pilV N-terminus as well as the previous rcideletion was amplified and used for a second round of recombieering again using pSIM6 in a MG1655Rf^{R} strain. The resulting strain TP114ΔpilV-rci::cat was then treated with pE-FLP, generating TP114ΔpilV-rci. Alternatively, a cassette containing a FLAG-tag and an FRT flanked cat gene was amplified from pKD3 (with the FLAG-tag being provided by the PCR primer). The cassette was inserted in TP114 to replace the 3′ end of pilV, the shufflon and the deleted shufflase gene region by recombineering (Datsenko et al., 2000). Recombinant clones of E. coli MG1655Rf^{R} were then screened using appropriate primers (Table 2). The deletion generated TP114pilVΔshufflon-rci::catin which the shufflon is replaced by a FLAG-tag. EachC-terminal variants of pilV were also amplified by PCR and fused to an FRT flanked chloramphenicol resistance cassette. The complete cassette contained homology regions for the pilV gene and the shufflase deletion scar. Recombineering using these cassettes generated “locked” configurations for each pilV variants (TP114pilVΔshufflon::pilV1-cat, TP114pilVΔshufflon::pilV2-cat, TP114pilVΔshufflon::pilV3-cat, TP114pilVΔshufflon::pilV3′-cat, TP114pilVΔshufflon::pilV4-cat, TP114pilVΔshufflon::pilV4′-cat, TP114pilVΔshufflon::pilV5-cat, TP114pilVΔshufflon::pilV5′-cat). Mutant versions of TP114, including TP114ΔpilV-rci, TP114ΔpilVΔshufflon-rci::cat, and the variants of the pilV adhesins (TP114pilVΔshufflon::pilV1-cat, TP114pilVΔshufflon::pilV2-cat, TP114pilVΔshufflon::pilV3-cat, TP114pilVΔshufflon::pilV3′-cat, TP114pilVΔshufflon::pilV4-cat, TP114pilVΔshufflon::pilV4′-cat, TP114pilVΔshufflon::pilV5-cat, TP114pilVΔshufflon::pilV5′-cat) were transferred to E. coli strain KN01. The ability of the wildtype TP114 and its pilV mutant versions of TP114 to transfer from E. coli KN01 to E. coli KN03 was assayed under solid, liquid (static) and liquid with agitation conditions.

Construction and use of pPilS and pPilV4′. Plasmid pPilS was constructed by amplifying the pilS gene from TP114, oriV_{p15A}-araC-P_{BAD} from pBAD30 and cat from pSB1C3 using primers listed in Table 2 and joining them by Gibson assembly. Plasmid pPilS was then transformed into KN01+TP114ΔpilS for complementation studies. In a similar way, plasmid pPilV4′ was constructed by amplifying the pilV4′ gene from TP114, oriV_{p15A}-araC-P_{BAD} from pBAD30 and cat from pSB1C3 using primers listed in Table 2 and joining them by Gibson assembly. Plasmid pPilV4′ was then transformed into KN01+TP114ΔpilV for complementation studies. The pilS and pilV4′ genes are under the regulation of AraC⁴⁰, providing arabinose inducible expression. For complementation experiments, donor and recipient strains were grown overnight at 37° C. Two hours before conjugation, arabinose was added to the donor strain cultures at a final concentration of 1% w/v. Then, OD₆₀₀ₙₘ of each culture was measured and cells were washed in LB+1% arabinose then resuspended in a volume equivalent to 40 OD₆₀₀ₙₘ in LB+1% arabinose. A volume of 2.5 μL of the donor and recipient strains were then mixed together and deposited on an LB+1% arabinose plate for solid conjugation or mixed with 195 μL of pre-warmed LB+1% arabinose for conjugation under both liquid static and liquid shaking conditions. Matings were then performed at 37° C. for 2 hours. Additionally, conjugations under the liquid shaking condition were placed on a rotary agitator. After incubation, the matings were serially diluted 1/10 and plated on selective media for CFU analysis of the donor, recipient, and transconjugant strains.

In vitro conjugation assay. All in vitro conjugation experiments were performed as described in the Material and Method section of Example I. Of note, for liquid mating with agitation, cell mixes were incubated at 37° C. for 2 hours on a rotary mixer instead of the standard static incubation.

High-density transposon mutagenesis (HDTM). A conjugation assisted random transposon mutagenesis experiment was performed. The transposition system was composed of pFG036 (a plasmid coding for a cl transcription repressor), pFG051 (a pir-dependent suicide plasmid coding for the Tn5 transposon machinery under the repression of cl, a RP4-based origin of transfer and a Sp^{R} transposon) and MFDpir+ (Ferrières et al., 2010) (which has an RP4 conjugative machinery, diaminopimelic acid auxotrophy and the Pi protein necessary for pFG051's maintenance in the cell). The HDTM experiment was performed in several successive steps in order to clearly identify the function of genes involved at each one of these steps. First, pFG051 was transferred by conjugation from MFDpir+ to EcN containing TP114 for 2 hours at 30° C. on LB+DAP plates in triplicates. Once in EcN, Tn5 machinery was expressed from pFG051 to mediate random transposon insertions in TP114. Then, transconjugants were entirely plated onto 6 plates per replicates and incubated overnight at 37° C. After the incubation, transconjugant clones formed a cell lawn that was collected using a cell scrapper and subsequently resuspended in LB broth with selective antibiotics. Transconjugants, which forms the mutant library, were then washed, resuspended in 4.5 mL of LB+25% glycerol and frozen for storage. Also, 100 μL of the mutant library was used in two subsequent conjugative transfer experiments towards KN02 and then towards KN03, which were both carried in parallel in vitro and in vivo.

Mouse model for in vivo HDTM library conjugation. Mice related experiments were done as described in the Material and Method section of Example I with only minor modifications. The donor strain inoculum was prepared 3 to 4 hours prior to mice oral gavage. 500 μL of a frozen stock of the High-Density Transposon Mutagenesis (HDTM) mutant library was inoculated in 20 mL selective LB broth and incubated at 37° C. for 4 hours before gavage. When ready, cells were washed once in PBS and concentrated in a volume equivalent to 6.0 OD₆₀₀ₙₘ. Mice were orally challenged with the recipient strain 3 hours prior to the introduction of the donor strain. Conjugation was then monitored by feces sampling at 24 and 48 hours. At 48 hours, mice were sacrificed and the caecum was extracted. Also, for transconjugants, 4×100 μL per mice were also plated in order to obtain a large number of transconjugant clones for the sequencing.

HDTM libraries sequencing. For each sample, a 1.5 mL frozen stock aliquot of mutant library was thawed on ice for 15 minutes. The aliquot was centrifugated and cells were resuspended in 300 μL of Cell lysis buffer from the Quick gDNA Miniprep kit (ZymoResearch). DNA was fragmented using a Bioruptor Plus (Diagenode) for 12 cycles of 30 seconds ON, 30 seconds OFF at 4° C. After fragmentation, the Quick gDNA Miniprep kit's protocol for cell suspension was followed and DNA was eluted in 50 μL of molecular grade water. 10 μg of DNA was then end-repaired using End-repair Mix HC (Enzymatics) followed by DNA purification using AMPure DNA XP magnetic beads (Agencourt). Purified DNA was then adenylated using TaqB (Enzymatics) supplemented with dATP for 30 minutes at 68° C. and purified again with AMPure DNA XP beads (Agencourt). Nextera adaptator B was then generated by annealing two oligonucleotides: 5′-PO₄—CTGTCTCTTATACACATCTCCGAGCCCACGAGAC-InvdT-3′ (SEQ ID NO: 91) and 5′-CAAGCAGAAGACGGCATACGAGATTCGCCTTAGTCTCGTGGGCTCGGAGATGTGTATA AGAGACAGT-3′ (SEQ ID NO: 92) together. Annealing was performed by heating 40 μM of each oligonucleotide in annealing buffer (10 mM Tris NaCl pH 7.5, 50 mM NaCl) to 98° C. and then slowly decreasing 0.1° C. each 10 seconds until 4° C. was reached. Nextera adaptator B was ligated using T4 DNA ligase (Enzymatics) overnight at 16° C. DNA was purified again using DNA Ampure XP beads (Agencourt) and barcoding was performed in a qPCR machine using Veraseq DNA polymerase (Enzymatics). Amplification reaction was stopped at the end of the exponential phase. DNA was purified again and quantified using Quant-it PicoGreen DNA assay. Quality and size distribution of the amplified mutant library was assessed on Bioanalyzer using a High Sensitivity DNA Chip. Mutant libraries were then pooled and sequenced by Illumina using the Nextera technology.

HDTM mutant analysis. Reads were first trimmed based on their quality and the presence of the Nextera Illumina adapter using Trimmomatic, version 0.32, with the parameters SLIDINGWINDOW:4:20 and MINLEN:30 (Bolger et al., 2014). The quality of the reads, before and after trimming, was assessed with FastQC using the default parameters (Andrew, 2010). Reads mapping on EcN's chromosome were filtered out and the remaining reads were mapped onto TP114. These alignments were done with BWA MEM using the default parameters (Li, 2013). Alignments with a mapping quality score lower than 30 were discarded. The position of the middle base pair of the 9 bp Tn5 insertion site duplication was then used to represent every corresponding alignment (Goryshin et al., 1998). Insertion sites only represented by one read were discarded in an attempt to filter out sequencing noise. The insertion maps with normalized reads count (based on the library size) were then visualized using UCSC Genome Browser in a Box (Haeussler et al., 2015). The essentiality of the genes in condition 1 was verified manually, searching for low coverage regions that were mappable and reproducible in all of the three replicates. A gene count table was then generated by calculating the normalized read count (based on library size) of each TP114's gene for each condition. Insertion sites in the first 5% and last 15% of the gene were not considered in the read count as they may lead to functional gene fragments. The genes important for in vitro and in vivo conjugation were determined based on the gene read count ratio between condition 1 and the test condition. The formula used to compute the gene read count ratios is: (Read count x−Read count 1)/Read count 1. A core set of genes which were considered to be essential for conjugation in vitro (traABCDEGHIJK, trbJ, nikAB) and in vivo (pilLNOPQRSUV) were then used to set the maximal ratio value for each condition. All genes with gene count ratios below the maximal value were considered essential in the given condition.

2.1—TP114 Conjugative Plasmid Comparative Genomics

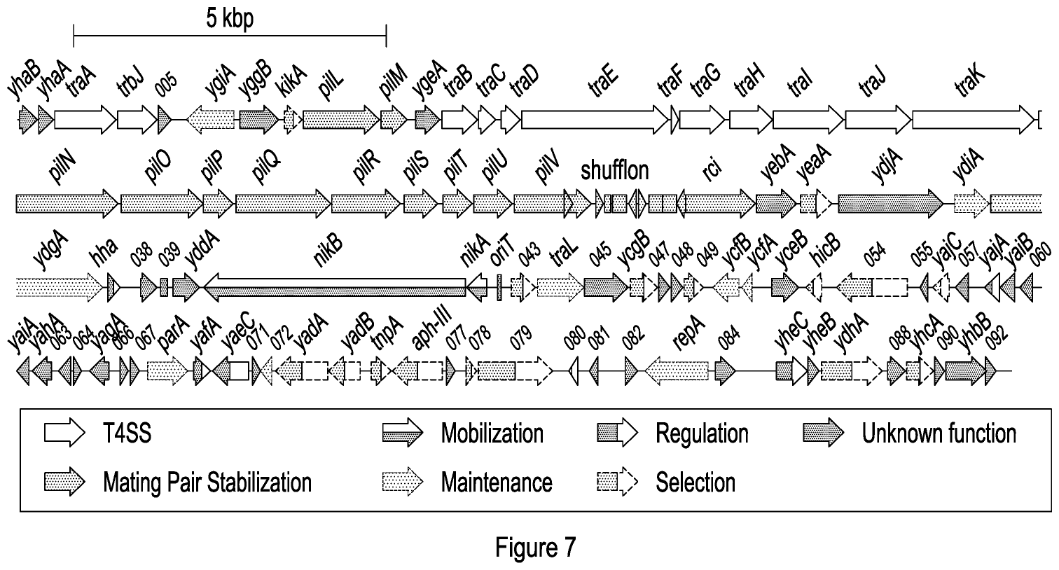

TP114 sequencing and annotation. In Example I, TP114 was identified to be the most potent conjugative plasmid for DNA cargo delivery in vivo. Therefore, it was the most interesting plasmid to be used as transfer machinery for the COP system. However, little is known about TP114. The first step toward the comprehension of TP114's transfer efficiency in vivo was thus to determine its complete sequence. TP114 was sequenced within an E. coli MG1655 strain using Illumina and Oxford Nanopore sequencing technologies. Sequence was then assembled in several ways including reference mapping onto related plasmid R721 from the IncI2 plasmid family and de novo sequence assembly. Then, the plasmid was automatically annotated using RAST to find potential ORFs. Annotation was then rectified by comparing them to annotations from R721 based on sequence homology. Genes with over 98% nucleotide homology to a gene on R721 were re-annotated to be consistent between plasmids. TP114's full sequence and annotation was then submitted to Genbank under accession number: MF521836.1. Plasmid TP114 is 64,818 bp long containing 92 CDS and has an average G+C proportion of 43%. TP114's genes were further characterized using BLASTn and CDsearchto find functional homologs. As conjugative plasmids tend to be modular, each gene was then attributed to a specific module with a specific function (type IV secretion system (T4SS), mating pair stabilization, maintenance, regulation, selection and unknown function). Genes were then mapped onto TP114 to generate a first graphical map of TP114's genes (FIG. 7).

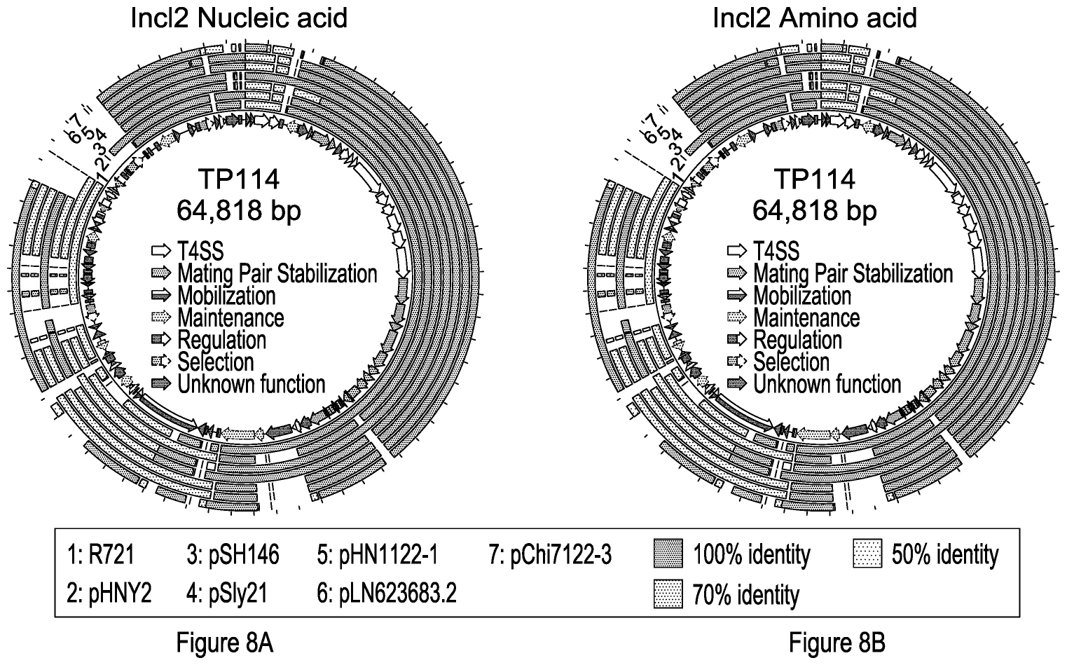

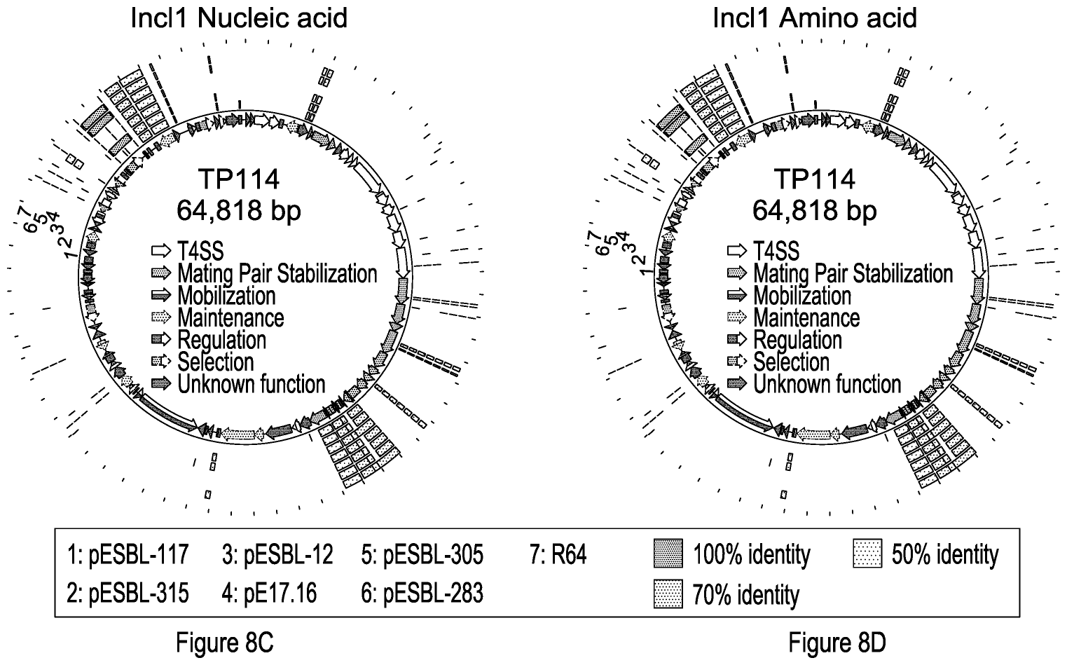

TP114 gene conservation analysis. One way to determine the importance of a specific gene present on a conjugative plasmid is to analyse its conservation. The conservation of genes can be evaluated by sequence homology against closely related conjugative plasmids. Fortunately, conjugative plasmids have been categorized in incompatibility families based on their ability to be stably maintained in the same cell or to be targeted by the same bacteriophage. The inability of two plasmids to share a same host is often linked to similarity between replication protein sequences. As such, since the primary sequence of TP114's replication protein is highly similar to the one of R721, which belongs to the IncI2 plasmid subfamily, TP114 was classified as an IncI2 plasmid. The IncI plasmid family is divided in two subfamilies, IncI1 and IncI2 and it is still unclear how much both groups share sequence homology. Therefore, comparative genomics analysis was carried on both plasmid subfamilies. Seven plasmids of both IncI1 and IncI2 subfamilies were selected based on the availability of their full genome sequence in Genbank (NCBI) (Table 5). These plasmids were then used as database for homology analysis with TP114 using the stand-alone BRIG software. TP114's genes were mostly highly conserved throughout the IncI2 plasmids both at the nucleic acid and amino acid levels (FIG. 8.A and FIG. 8.B). This suggested that most IncI2 plasmids could share TP114's ability to transfer DNA efficiently in vivo. Homology towards IncI1 plasmids was really scarce both at the nucleic acid and amino acid levels (FIG. 8.0 and FIG. 8.D). Even though gene functions are similar between IncI1 and IncI2 groups, high sequence divergence was found between these two subfamilies. Therefore, for the analysis of gene conservation, only the IncI2 subfamily had been considered since sequence homology with the IncI1 group was too low. Genes were categorized into 3 groups: core, soft core and accessory (Table 6). Core genes were conserved in all plasmids of the IncI2 family whereas the soft-core genes were conserved in >50% of the plasmids. Finally, genes were considered accessory if they were present in <50% of the plasmids.

TABLE 5
Plasmids used in the comparative genomic analysis.

| | Incompatibility | Lenght | NCBI accession |
|---|---|---|---|
| Plasmid name | group | (bp) | number |
| pESBL-117 | Incl1 | 89503 | CP008734 |
| pESBL-315 | Incl1 | 93037 | CP008738 |
| pESBL-12 | Incl1 | 96463 | CP008735 |
| pE17.16 | Incl1 | 101321 | CP008733 |
| pESBL-305 | Incl1 | 107552 | CP008737 |
| pESBL-283 | Incl1 | 110137 | CP008736 |
| R64 | Incl1 | 120826 | NC_005014.1 |
| TP114 | Incl2 | 64818 | MF521836.1 |
| pChi7122-3 | Incl2 | 56676 | FR851304 |
| LN623683.2 | Incl2 | 62139 | LN623683.2 |
| pHN1122-1 | Incl2 | 62196 | JN797501 |
| pSly21 | Incl2 | 63329 | NZ_CP016405.1 |
| pSH146_65 | Incl2 | 65030 | JN983044 |
| pHNY2 | Incl2 | 65358 | KF601686.2 |
| R721 | Incl2 | 75582 | AP002527.1 |

TABLE 6
TP114 in silico gene function prediction by CDsearch and BLAST.

| | | name in | Protein family/ | Known homolog/ | |
|---|---|---|---|---|---|
| module | Locus tag | TP114 | Identity | Function | Conservation |
| T4SS | TP114-003 | traA | pfam04610 | virB6 | Core |
| | TP114-004 | trbJ | pfam07996 | virB5 | Core |
| | TP114-012 | traB | pfam01464 | virB1 | Core |
| | TP114-013 | traC | BLASTx 99% identity | virB2 | Core |
| | TP114-014 | traD | pfam05101 | virB3 | Core |
| | TP114-015 | traE | pfam03135 | virB4 | Core |
| | TP114-016 | traF | BLASTx 100% identity | R721 traF (virB7) | Core |
| | TP114-017 | traG | pfam04335 | virB8 | Core |
| | TP114-018 | traH | pfam03524 | virB9 | Core |
| | TP114-019 | traI | pfam03743 | virB10 | Core |
| | TP114-020 | traJ | pfam00437 | virB11 | Core |
| | TP114-021 | traK | pfam02534 | virD4 | Core |
| Mating Pair | TP114-009 | pilL | pfam10671 | tcpQ | Core |
| Stabilization | TP114-010 | pilM | pfam07419 | pilM | Core |
| | TP114-022 | pilN | TIGR02520 | pilN | Core |
| | TP114-023 | pilO | pfam06864 | pilO | Core |
| | TP114-024 | pilP | TIGR03021 | pilP | Core |
| | TP114-025 | pilQ | pfam00437 | virB11 | Core |
| | TP114-026 | pilR | pfam00482 | T2SSF | Core |
| | TP114-027 | pilS | pfam08805 | pilS | Core |
| | TP114-028 | pilT | cl00222 | virB1 | Core |
| | TP114-029 | pilU | pfam01478 | Peptidase_A24 | Core |
| | TP114-030 | pilV | pfam04917 | Shufflon_N | Core |
| | TP114-031 | rci | pfam00589 | Phage integrase | Core |
| Mobilization | TP114-041 | nikB | pfam03432 | Relaxase | Core |
| | TP114-042 | nikA | pfam05713 | mobC | Core |
| | TP114-068* | parA | cd02042 | virC1 | Core |
| maintenance | TP114-006 | ygiA | BLASTx 98% identity | DNA primase | Soft-core |
| | TP114-035 | ydiA | BLASTx 99% identity | DNA topoisomerase III | Accessory |
| | TP114-036 | ydgA | cl27597 | DNA topoisomerase IA | Core |
| | TP114-044 | traL | cl27521 | parA | Soft-core |
| | TP114-050 | ycfB | pfam15919 | hicB Anti-toxin | Core |
| | TP114-051 | ycfA | pfam07927 | hicA toxin | Core |
| | TP114-068* | parA | cd02042 | parA | Core |
| | TP114-072 | TP114-072 | cl10143 | Post-Segregation killing protein | Core |
| | TP114-083 | repA | pfam02387 | repA | Core |
| regulatory | TP114-037 | hha | pfam05321 | Haemolysin expression modulating protein | Core |
| | TP114-058 | yajA | pfam15731 | Transcription regulator | Accessory |
| | TP114-069 | yafA | BLASTx 99% identity | cogG Transcription regulator | Core |
| | TP114-070 | yaeC | cd00236 | finO Transcription repressor | Core |
| | TP114-085 | yheC | pfam07180 | CaiF/GrIA transcriptional regulator | Core |
| selection | TP114-008 | kikA | pfam07424 | p-toluenesulfonate degradation (trbM) | Core |
| | TP114-033 | yeaA | pfam00565 | Endonuclease | Core |
| | TP114-043 | TP114-043 | BLASTx 98% identity | Putative zinc transporter | Soft-core |
| | TP114-046 | ycgB | BLASTx 98% identity | valyl-tRNA synthase | Accessory |
| | TP114-049 | TP114-049 | BLASTx 88% identity | valyl-tRNA synthase | Core |
| | TP114-053 | hicB | pfam05534 | Rnase H | Accessory |
| | TP114-054 | TP114-054 | pfam01385 | Transposase | Accessory |
| | TP114-056 | yajC | BLASTx 99% identity | cobalamin biosynthesis (cibX) | Accessory |
| | TP114-073 | yadA | cl26233 | Transposase | Accessory |
| | TP114-074 | yadB | cl26146 | Transposase | Accessory |
| | TP114-075 | tnpA | BLASTx 100% identity | Tn3 familly transposase | Accessory |
| | TP114-076 | aph-III | cd05150 | Aminoglycoside 3′-phosphotransferase | Accessory |
| | TP114-078 | TP114-078 | BLASTx 91% identity | Transposase | Accessory |
| | TP114-079 | TP114-079 | pfam04986 | Transposase | Accessory |
| | TP114-080 | TP114-080 | BLASTx 100% identity | IS91 transposase | Core |
| | TP114-087 | ydhA | cd08826 | Stomatin-like protein | Core |
| | TP114-089 | yhcA | cl00686 | Regulation of membrane protease activity | Core |
| Unknown | TP114-001 | yhaB | None | Unknown | Core |
| function | TP114-002 | yhaA | None | Unknown | Core |
| | TP114-005 | TP114-005 | None | Unknown | Accessory |
| | TP114-007 | yggB | None | Unknown | Core |
| | TP114-011 | ygeA | None | Unknown | Core |
| | TP114-032 | yebA | None | Unknown | Core |
| | TP114-034 | YdjA | None | Unknown | Accessory |
| | TP114-038 | TP114-038 | None | Unknown | Accessory |
| | TP114-039 | TP114-039 | None | Unknown | Accessory |
| | TP114-040 | yddA | None | Unknown | Core |
| | TP114-045 | TP114-045 | None | Unknown | Soft-core |
| | TP114-047 | TP114-047 | None | Unknown | Accessory |
| | TP114-048 | TP114-048 | None | Unknown | Accessory |
| | TP114-052 | yceB | None | Unknown | Core |
| | TP114-055 | TP114-055 | None | Unknown | Core |
| | TP114-057 | TP114-057 | None | Unknown | Core |
| | TP114-059 | yaiB | None | Unknown | Accessory |
| | TP114-060 | TP114-060 | None | Unknown | Core |
| | TP114-061 | yaiA | None | Unknown | Accessory |
| | TP114-062 | yahA | None | Unknown | Accessory |
| | TP114-063 | TP114-063 | None | Unknown | Soft-core |
| | TP114-064 | TP114-064 | None | Unknown | Core |
| | TP114-065 | yagA | None | Unknown | Core |
| | TP114-066 | TP114-066 | None | Unknown | Core |
| | TP114-067 | TP114-067 | None | Unknown | Core |
| | TP114-071 | TP114-071 | None | Unknown | Core |
| | TP114-077 | TP114-077 | None | Unknown | Accessory |
| | TP114-081 | TP114-081 | None | Unknown | Core |
| | TP114-082 | TP114-082 | None | Unknown | Core |
| | TP114-084 | TP114-084 | None | Unknown | Core |
| | TP114-086 | yheB | pfam06688 | Unknown | Core |
| | TP114-088 | TP114-088 | None | Unknown | Accessory |
| | TP114-090 | TP114-090 | None | Unknown | Core |
| | TP114-091 | yhbB | None | Unknown | Soft-core |
| | TP114-092 | TP114-092 | None | Unknown | Soft-core |

*This gene might have a function in more than one module.

TP114 encoded for a mating pair stabilization module. One interesting feature shared by I-complex plasmids (IncB/O (Inc10), IncI1, IncI2, IncK and incZ alike) is the presence of genes encoding a functional type IV pilus (T4P) (Sekizuka et al., 2017). As observed with other plasmids from the I-complex, TP114 encoded a T4P that was independent from the traditional T4SS. Such an apparatus is thought to improve mating pair stabilization (hereby named mating pair stabilization module) by binding directly to the recipient's membrane and retracting the pilus to facilitate donor/recipient direct contact (Bradley, 1984). Very few plasmid families are known to encode T4P (e.g. IncI1 (Ishiwa et al., 2003), IncI2 (Sekizuka et al., 2017), IncB/O (Inc10) (Papagiannitsis et al., 2011), IncK (Seiffert et al., 2017), and IncZ (Venturini et al., 2013)).

2.2—High Density Transposon Mutagenesis of TP114.

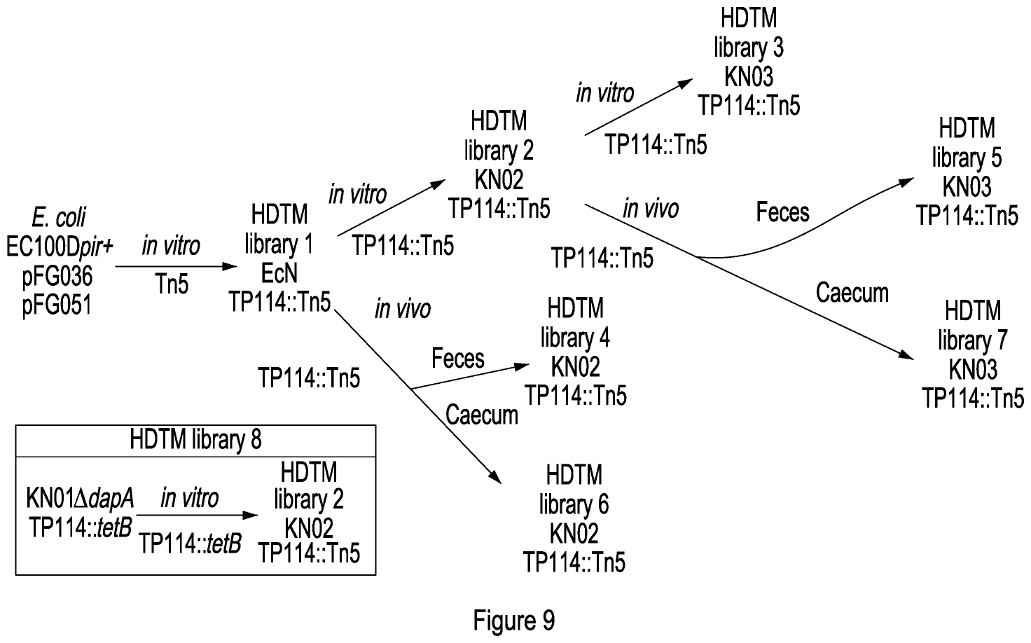

HDTM experiment design. Several HDTM experiments were needed to fully characterize TP114 genes functions. As such, a scheme describing the HDTM is presented to fully comprehend the extent of the experiment (FIG. 9). HDTM step 1 generated a full library of TP114 mutants. At this point, if a transposon interrupts a gene required for short-term plasmid maintenance in the cell, this would lead to plasmid loss. By using antibiotics to select for TP114 and the Tn5 transposon, cells that have lost the plasmid will not survive. Therefore, insertion of Tn5 within essential maintenance genes will prevent those genes from being sequenced resulting on low coverage of their loci. This HDTM library 1 was then used in two different conjugation experiments, one where the TP114::Tn5 were transferred by solid mating in vitro (Step 2) and one where the mating was carried in vivo (Step 4 where transconjugants originated from the feces and Step 6 where the transconjugants were retrieved from the caecum). The HDTM library 2 revealed genes essential for conjugation in vitro whereas HDTM library 4 and 6 revealed genes essential for conjugation in vivo, which allowed us to discriminate between genes required in both environments and genes only required in one specific environment. The HDTM library 2 was further used as donors in two supplementary mating experiments in vitro (Step 3) and in vivo (Step 5 for feces extracted transconjugants and Step 7 for caecum extracted transconjugants). This second transfer is expected to enrich genes which once inactivated by the Tn5 transposon have a positive effect on conjugation efficiency (e.g. a transcription repressor). Furthermore, a second transfer step ensured minimal background by diluting the initial donor cells. Finally, HDTM step 8 required the transfer of the modified TP114::tetB into HDTM library 2. Conjugative plasmids of a same incompatibility family can usually prevent the acquisition of another related plasmids through a mechanism called exclusion. Since TP114 can mediate exclusion, TP114::tetB can only enter the cell if the exclusion related gene(s) are interrupted by the Tn5 transposon.

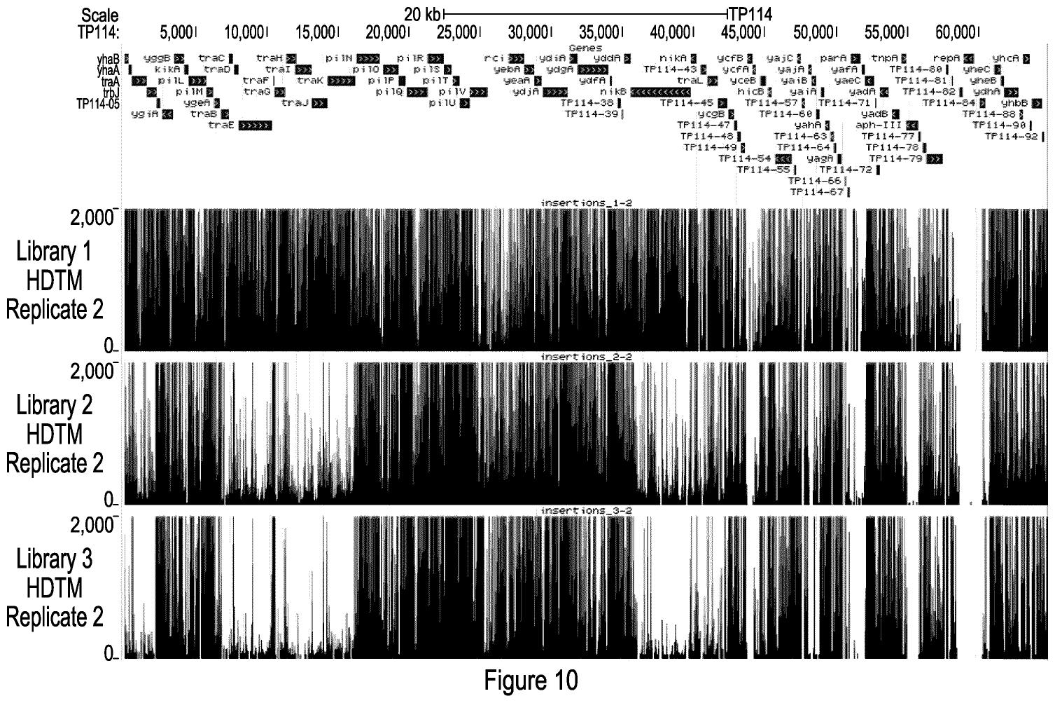

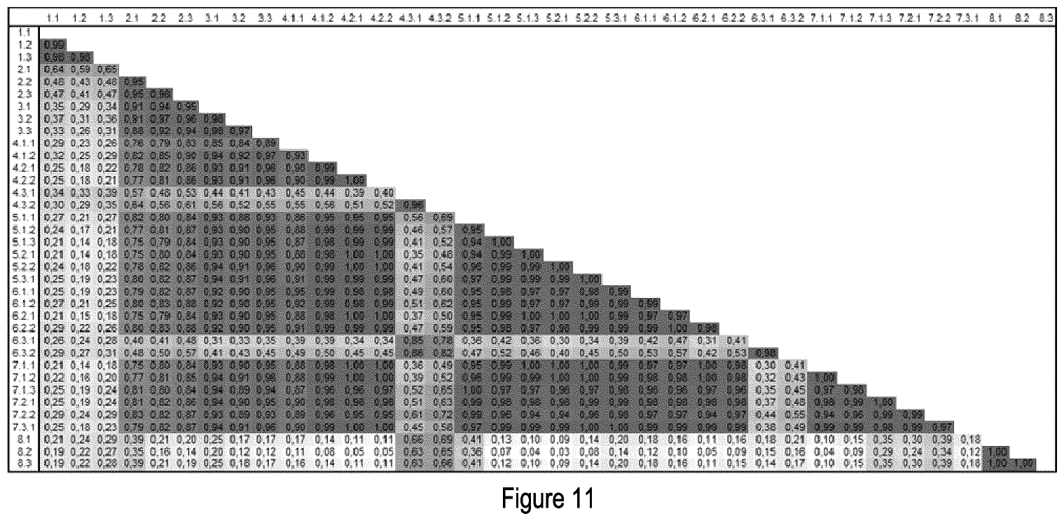

HDTM analysis consideration. Analysis of the HDTM mutant libraries revealed an average coverage of 9.68 insertions per bp in TP114. This high resolution allowed us to assess the essentiality of even the smallest annotated TP114 gene. However, TP114 encodes a set of 7 genes containing repeated regions in which reads cannot be mapped (termed 0-mappability regions) (Table 7). Those genes appeared under-represented, but were not necessarily essential and were analyzed by considering only the portion of the gene that was mappable. The HDTM experiment also accounted for possible donor DNA contamination. By doing successive transfer experiment with the HDTM library, background contamination was drastically reduced and showed consistent results. As such, a clear drop in background level was seen from HDTM library 2 to HDTM library 3 and consistent results were observed between libraries (FIG. 10). The HDTM analysis also took advantage of a high number of replicates and conditions to allow for comparison of read count between conditions and between biological replicates. Similarity between replicates and conditions was evaluated by Pearson Correlation and revealed well correlated replicates and weaker correlation between the different conditions (FIG. 11). This kind of distribution was expected as different conditions will put different selective pressure onto the population of mutants, selecting the mutants with the best fits for each specific condition. Only replicates from mice group C of HDTM library 4 and 6 was weakly correlated to others and was discarded from the analysis.

TABLE 7
0-mappability regions in TP114.

| Gene | Length (bp) | 0-map bp | % mappable |
|---|---|---|---|
| TP114-049 | 327 | 22 | 93.27% |
| TP114-057 | 216 | 156 | 27.78% |
| TP114-063 | 216 | 77 | 64.35% |
| TP114-064 | 147 | 70 | 52.38% |
| TP114-066 | 138 | 76 | 44.93% |
| TP114-067 | 165 | 67 | 59.39% |
| ycgB | 450 | 22 | 95.11% |

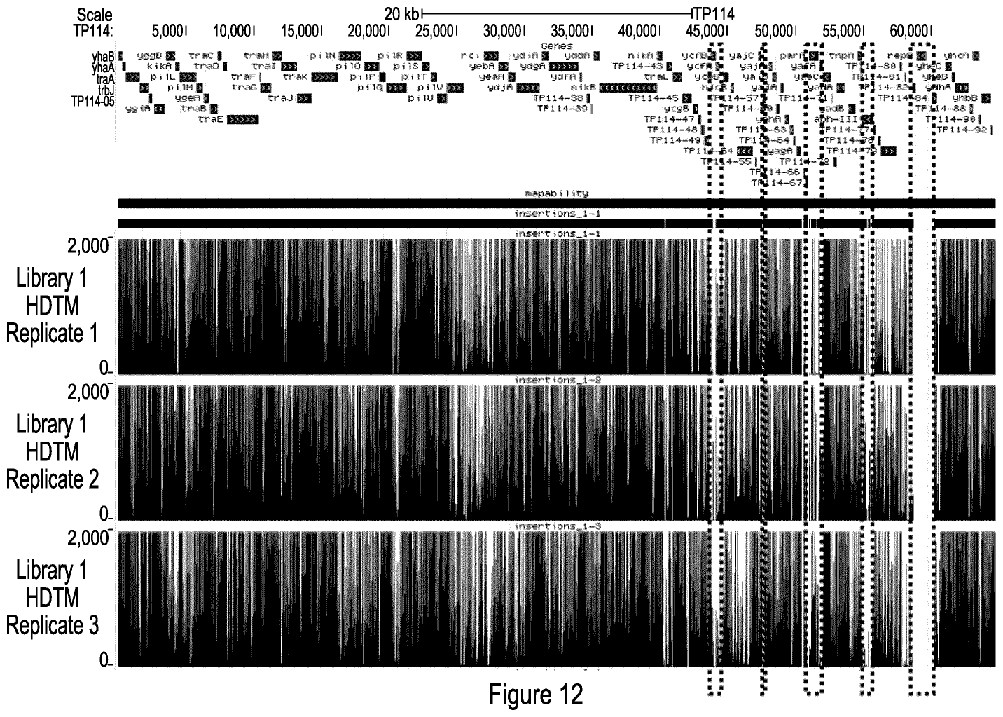

HDTM identified important features for TP114 replication and maintenance. The first step of HDTM was to generate a mutant library with insertion in all genes (Library 1). In this set-up, only insertions in the sequences important for the replication and maintenance of TP114 should produce non-viable clones. Therefore, genes important for replication should be underrepresented in the read coverage as compared to other genes. As suspected, most genes had high insertion coverage except for a core set of 6 genes which were reproducibly under-represented (FIG. 12). Among those, repA was already suspected to be a critical actor in plasmid maintenance, and the aph-Ill gene was used for transconjugant selection and therefore would appear essential. The list of essential genes for replication and maintenance is shown in Table 8. Most of these genes were already suspected to be part of the maintenance module or were highly conserved in the IncI2 family. However, ygiA (TP114-006), ydiA (TP114-035), ydgA (TP114-036), traL (TP114-044) and TP114-072 that were predicted to be implicated in plasmid maintenance were found to be dispensable by HDTM. There was a possibility that these genes were not required for plasmid maintenance in E. coli due to functional redundancy, but could be involved in plasmid maintenance in other host species. Some genes, like traL, might be implicated in another function of the conjugative plasmid. Nonetheless, the gene function prediction was mostly in agreement with the HDTM results.

TABLE 8
Revised list of essential maintenance genes.

| Predicted module | Locus tag | name in TP114 | Predicted Function | Conservation |
|---|---|---|---|---|
| maintenance | TP114-050 | ycfB | hicB Anti-toxin | Core |
| maintenance | TP114-051 | ycfA | hicA toxin | Core |
| maintenance | TP114-068 | parA | Partition | Core |
| selection | TP114-076 | aph-III | Kanamycin resistance | Accessory |
| unknown function | TP114-082 | TP114-082 | Unknown | Core |
| maintenance | TP114-083 | repA | Replication initiation | Core |

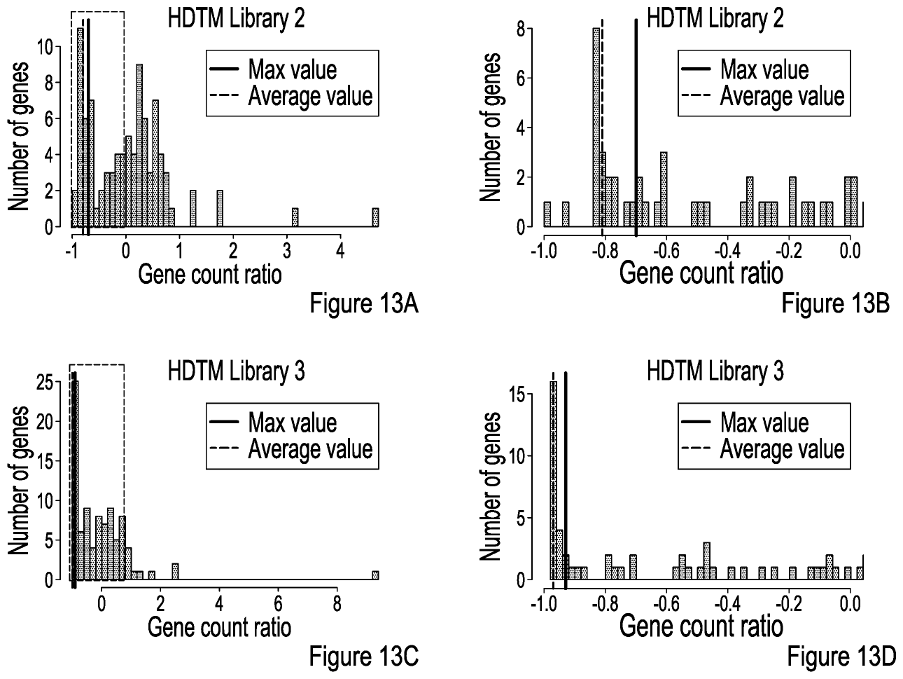

Identification of TP114 genes essential for in vitro conjugation on solid medium. Gene importance for in vitro mating on solid medium was evaluated by gene count ratios. Gene count ratios were calculated by comparing the number of reads that map in a given gene in two different contexts. Briefly, genes which became under-represented following conjugative transfer in vitro (libraries 2 and 3 as compared to library 1) gave negative gene count ratio. To assess gene essentiality and account for any bias, a set of gene, which were predicted to be essential for conjugation (traABCDEFGHIJK, trbJ, nikAB), was used to evaluate the maximal and average gene count ratio of essential genes. However, traF had a high gene count ratio and was considered an outlier and not essential for conjugation. Gene distribution was plotted for both HDTM library 2 and 3 (FIG. 13) and all genes bellow the maximum gene count ratio value were considered essential for conjugation in the given library. Genes that were already determined to be implicated in plasmid maintenance were discarded. The list of genes essential for TP114 conjugation in vitro is shown in Table 9.

TABLE 9
Important genes for in vitro conjugation.

| Predicted | | name | Predicted | | |
|---|---|---|---|---|---|
| module | Locus tag | in TP114 | Function | Conservation | Library |
| Unknown function | TP114-001 | yhaB | Unknown | Core | 2, 3 |
| Unknown function | TP114-002 | yhaA | Unknown | Core | 3 |
| T4SS | TP114-003 | traA | VirB6 | Core | 2, 3 |
| T4SS | TP114-004 | trbJ | VirB5 | Core | 2, 3 |
| Unknown function | TP114-011 | ygeA | Unknown | Core | 3 |
| T4SS | TP114-012 | traB | VirB1 | Core | 2, 3 |
| T4SS | TP114-013 | traC | VirB2 | Core | 2, 3 |
| T4SS | TP114-014 | traD | VirB3 | Core | 2, 3 |
| T4SS | TP114-015 | traE | VirB4 | Core | 2, 3 |
| T4SS | TP114-017 | traG | VirB8 | Core | 2, 3 |
| T4SS | TP114-018 | traH | VirB9 | Core | 2, 3 |
| T4SS | TP114-019 | traI | VirB10 | Core | 2, 3 |
| T4SS | TP114-020 | traJ | VirB11 | Core | 2, 3 |
| T4SS | TP114-021 | traK | VirD4 | Core | 2, 3 |
| Mobilization | TP114-041 | nikB | Relaxase | Core | 2, 3 |
| Mobilization | TP114-042 | nikA | mobC | Core | 2, 3 |
| Selection | TP114-043 | TP114-043 | Putative zinc | Soft-core | 2 |
| | | | transporter | | |
| Regulation | TP114-058 | yaj4 | Transcription | Accessory | 2, 3 |
| | | | regulator | | |
| Unknown function | TP114-060 | TP114-060 | Unknown | Core | 2, 3 |
| Regulation | TP114-069 | yafA | cogG Transcription | Core | 3 |
| | | | regulator | | |

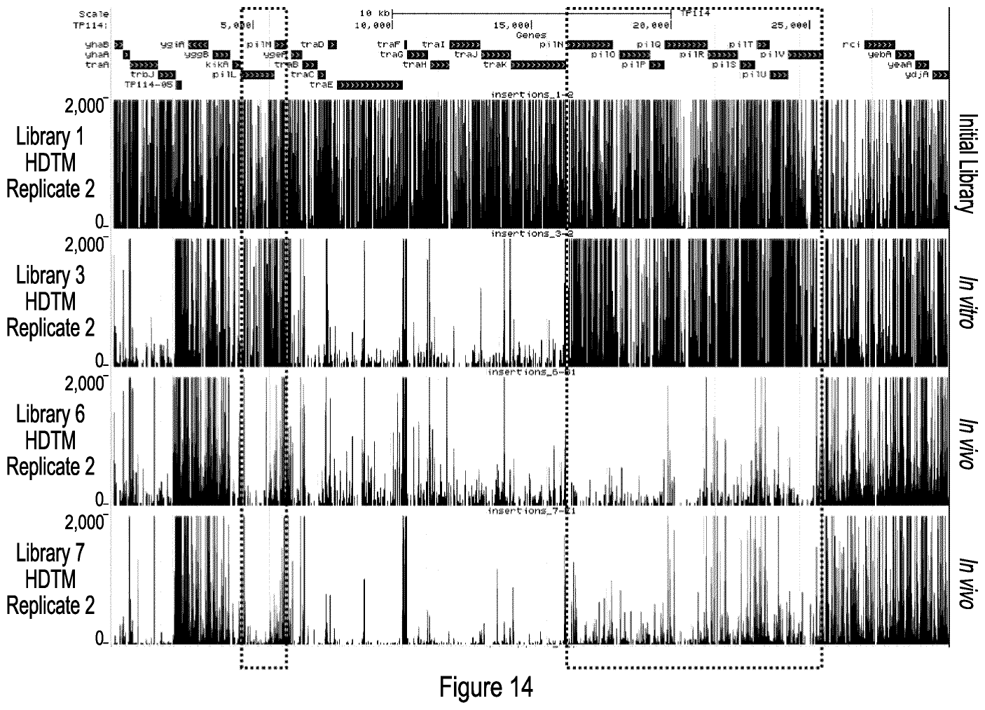

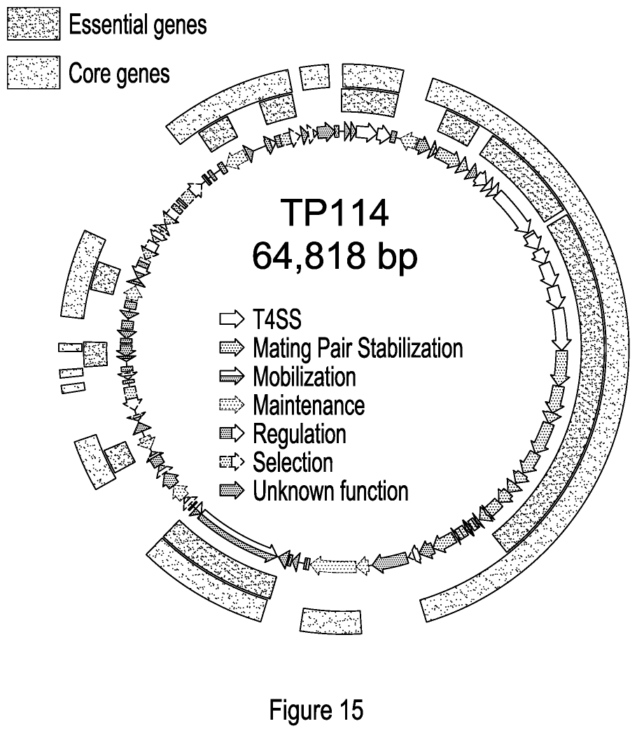

Identification of genes important for TP114 conjugation in vivo. Gene essentiality for in vivo conjugation of TP114 was carried out similarly to the in vitro analysis. However, the set used to fix the maximum gene count ratio was composed of pilLMNOPQRSTUV as it was apparent that pil genes were essential only for in vivo conjugation (FIG. 14). The pilM and pilT genes were removed from the set as they were outliers. It was critical to use genes which are suspected to be important only in vivo, at least for HDTM library 5 and 7. Indeed, the HDTM library 5 and 7 were generated by in vivo conjugation of HDTM library 2, which in turn is generated from an in vitro transfer of HDTM library 1. As such, since the first in vitro mating put a strong selective pressure for plasmids that can transfer in vitro and genes that are essential in vitro can also be essential in vivo, seeding with these genes excluded the majority of the genes that were only essential in vivo. Nonetheless, as 4 different conditions, including 12 total mice, were used for in vivo conjugation, gene essentiality can also be evaluated by consistency between replicates from the same or different conditions. Genes which were below the threshold for any of the in vivo conditions are listed in Table 10. However, genes which were already proven to be important for plasmid maintenance were not included in this table, as the gene count ratio would often lead to division by 0. When HDTM library 1 was used as donor cells for in vivo conjugation (conditions 4 and 6), 40 genes were found to be essential in the feces samples, and 36 in the caecum samples. Of these genes, 31 were shared in both conditions. Consistently, for the conjugation of HDTM library 2 in vivo (conditions 5 and 7), 40 genes were found to be important in feces samples and 37 genes were found to be important in the caecum samples, with 33 shared genes. In total, 40 genes were found to be important in at least one condition, of which 31 were shared in all conditions. Interestingly, most of the genes important for in vitro conjugation were also important for in vivo conjugation. As condition 5 and 7 follow an in vitro conjugation, this was expected. Additionally, most of pil genes were essential for in vivo transfer, with pilM and pilT being the only two exceptions.

TABLE 10
List of important genes for in vivo conjugation.

| Predicted | | name in | Predicted | | |
|---|---|---|---|---|---|
| module | Locus tag | TP114 | Function | Conservation | Library |
| Unknown function | TP114-001 | yhaB | Unknown | Core | 4, 5, 6, 7 |
| Unknown function | TP114-002 | yhaA | Unknown | Core | 4, 5, 6, 7 |
| T4SS | TP114-003 | traA | virB6 | Core | 4, 5, 6, 7 |
| T4SS | TP114-004 | trbJ | virB5 | Core | 4, 5, 6, 7 |
| selection | TP114-008 | kikA | p-toluenesulfonate | Core | 4, 5, 6 |
| | | | degradation (trbM) | | |
| Mating Pair | TP114-009 | pilL | tcpQ | Core | 4, 5, 6, 7 |
| Stabilization | | | | | |
| Unknown function | TP114-011 | ygeA | Unknown | Core | 4, 5, 6, 7 |
| T4SS | TP114-012 | traB | virB1 | Core | 5, 6, 7 |
| T4SS | TP114-013 | traC | virB2 | Core | 4, 5, 6, 7 |
| T4SS | TP114-014 | traD | virB3 | Core | 4, 5, 6, 7 |
| T4SS | TP114-015 | traE | virB4 | Core | 4, 5, 6, 7 |
| T4SS | TP114-017 | traG | virB8 | Core | 4, 5, 6, 7 |
| T4SS | TP114-018 | traH | virB9 | Core | 4, 5, 6, 7 |
| T4SS | TP114-019 | traI | virB10 | Core | 4, 5, 6, 7 |
| T4SS | TP114-020 | traJ | virB11 | Core | 4, 5, 6, 7 |
| T4SS | TP114-021 | traK | virD4 | Core | 4, 5, 6, 7 |
| Mating Pair | TP114-022 | pilN | pilN | Core | 4, 5, 6, 7 |
| Stabilization | | | | | |
| Mating Pair | TP114-023 | pilO | pilO | Core | 4, 5, 6, 7 |
| Stabilization | | | | | |
| Mating Pair | TP114-024 | pilP | pilP | Core | 4, 5, 6, 7 |
| Stabilization | | | | | |
| Mating Pair | TP114-025 | pilQ | virB11 | Core | 4, 5, 6, 7 |
| Stabilization | | | | | |
| Mating Pair | TP114-026 | pilR | T2SSF | Core | 4, 5, 6, 7 |
| Stabilization | | | | | |
| Mating Pair | TP114-027 | pilS | pilS | Core | 4, 5, 6, 7 |
| Stabilization | | | | | |
| Mating Pair | TP114-029 | pilU | Peptidase_A24 | Core | 4, 5, 6, 7 |
| Stabilization | | | | | |
| Mating Pair | TP114-030 | pilV | Shufflon_N | Core | 4, 5, 6, 7 |
| Stabilization | | | | | |
| Mobilization | TP114-041 | nikB | Relaxase | Core | 4, 5, 6, 7 |
| Mobilization | TP114-042 | nikA | mobC | Core | 4, 5, 6, 7 |
| selection | TP114-043 | TP114-43 | Putative zinc | Soft-core | 5, 7 |
| | | | transporter | | |
| maintenance | TP114-044 | traL | parA | Soft-core | 4, 5, 7 |
| Regulation | TP114-058 | yajA | Transcription | Accessory | 4, 5, 6, 7 |
| | | | regulator | | |
| Unknown function | TP114-059 | yaiB | Unknown | Accessory | 4, 5, 6, 7 |
| Unknown function | TP114-060 | TP114-060 | Unknown | Core | 4, 5, 6, 7 |
| Regulation | TP114-069 | yafA | cogG | Core | 4, 5, 6, 7 |
| | | | Transcription | | |
| | | | regulator | | |
| Regulation | TP114-085 | yheC | CaiF/GrlA | Core | 4 |
| | | | transcriptional | | |
| | | | regulator | | |
| Unknown function | TP114-086 | yheB | Unknown | Core | 4, 5, 6, 7 |
| selection | TP114-087 | ydhA | Stomatin-like | Core | 4, 5, 6, 7 |
| | | | protein | | |
| Unknown function | TP114-088 | TP114-088 | Unknown | Accessory | 4 |
| selection | TP114-089 | yhcA | Regulation of | Core | 4 |
| | | | membrane | | |
| | | | protease activity | | |
| Unknown function | TP114-090 | TP114-090 | Unknown | Core | 5 |
| Unknown function | TP114-091 | yhbB | Unknown | Soft-core | 5 |
| Unknown function | TP114-092 | TP114-092 | Unknown | Soft-core | 4 |

TP114 possessed a core set of genes that were important for conjugation. Performing HDTM with a high number of biological replicates allowed us to attribute confidence level to the importance of each gene for different functions of TP114 (Table 11). The confidence level was based on reproducibility of the result, with ++ being the highest confidence level and − being the lowest. For plasmid maintenance, confidence level was attributed differently than other conditions. A confidence level of ++ meant that the genes were essential in all replicates and − meant it was not essential in at least one condition. For all other conditions, confidence level is based on the degrees of reproducibility between conditions, with ++ meaning the gene was essential in all conditions, + meaning it was essential in only one of the conditions and − meaning it was never essential

TABLE 11
Confidence level of the importance of TP114 genes for conjugation and maintenance

| | | Essentiality confidence level | | | | |
|---|---|---|---|---|---|---|
| Locus | name in | | in vitro | in vivo | in vitro and in vivo | |
| tag | TP114 | maintenance | conjugation | conjugation only | conjugation | Conservation |
| TP114- | yhaB | − | ++ | ++ | ++ | Core |
| 001 | | | | | | |
| TP114- | yhaA | − | + | ++ | ++ | Core |
| 002 | | | | | | |
| TP114- | traA | − | ++ | ++ | ++ | Core |
| 003 | | | | | | |
| TP114- | trbJ | − | ++ | ++ | ++ | Core |
| 004 | | | | | | |
| TP114- | TP114- | − | − | − | − | Accessory |
| 005 | 005 | | | | | |
| TP114- | ygiA | − | − | − | − | Soft-core |
| 006 | | | | | | |
| TP114- | yggB | − | − | − | − | Core |
| 007 | | | | | | |
| TP114- | kikA | − | − | ++ | + | Core |
| 008 | | | | | | |
| TP114- | pilL | − | − | ++ | ++ | Core |
| 009 | | | | | | |
| TP114- | pilM | − | − | − | − | Core |
| 010 | | | | | | |
| TP114- | ygeA | − | + | ++ | ++ | Core |
| 011 | | | | | | |
| TP114- | traB | − | ++ | + | ++ | Core |
| 012 | | | | | | |
| TP114- | traC | − | ++ | ++ | ++ | Core |
| 013 | | | | | | |
| TP114- | traD | − | ++ | ++ | ++ | Core |
| 014 | | | | | | |
| TP114- | traE | − | ++ | ++ | ++ | Core |
| 015 | | | | | | |
| TP114- | traF | − | − | − | − | Core |
| 016 | | | | | | |
| TP114- | traG | − | ++ | ++ | ++ | Core |
| 017 | | | | | | |
| TP114- | traH | − | ++ | ++ | ++ | Core |
| 018 | | | | | | |
| TP114- | traI | − | ++ | ++ | ++ | Core |
| 019 | | | | | | |
| TP114- | traJ | − | ++ | ++ | ++ | Core |
| 020 | | | | | | |
| TP114- | traK | − | ++ | ++ | ++ | Core |
| 021 | | | | | | |
| TP114- | pilN | − | − | ++ | ++ | Core |
| 022 | | | | | | |
| TP114- | pilO | − | − | ++ | ++ | Core |
| 023 | | | | | | |
| TP114- | pilP | − | − | ++ | ++ | Core |
| 024 | | | | | | |
| TP114- | pilQ | − | − | ++ | ++ | Core |
| 025 | | | | | | |
| TP114- | pilR | − | − | ++ | ++ | Core |
| 026 | | | | | | |
| TP114- | pilS | − | − | ++ | ++ | Core |
| 027 | | | | | | |
| TP114- | pilT | − | − | − | − | Core |
| 028 | | | | | | |
| TP114- | pilU | − | − | ++ | ++ | Core |
| 029 | | | | | | |
| TP114- | pilV | − | − | ++ | ++ | Core |
| 030 | | | | | | |
| TP114- | rci | − | − | − | − | Core |
| 031 | | | | | | |
| TP114- | yebA | − | − | − | − | Core |
| 032 | | | | | | |
| TP114- | yeaA | − | − | − | − | Core |
| 033 | | | | | | |
| TP114- | YdjA | − | − | − | − | Accessory |
| 034 | | | | | | |
| TP114- | ydiA | − | − | − | − | Accessory |
| 035 | | | | | | |
| TP114- | ydgA | − | − | − | − | Core |
| 036 | | | | | | |
| TP114- | hha | − | − | − | − | Core |
| 037 | | | | | | |
| TP114- | TP114- | − | − | − | − | Accessory |
| 038 | 038 | | | | | |
| TP114- | TP114- | − | − | − | − | Accessory |
| 039 | 039 | | | | | |
| TP114- | yddA | − | − | − | − | Core |
| 040 | | | | | | |
| TP114- | nikB | − | ++ | ++ | ++ | Core |
| 041 | | | | | | |
| TP114- | nikA | − | ++ | ++ | ++ | Core |
| 042 | | | | | | |
| TP114- | TP114- | − | + | − | ++ | Soft-core |
| 043 | 043 | | | | | |
| TP114- | traL | − | − | + | ++ | Soft-core |
| 044 | | | | | | |
| TP114- | TP114- | − | − | − | − | Soft-core |
| 045 | 045 | | | | | |
| TP114- | ycgB | − | − | − | − | Accessory |
| 046 | | | | | | |
| TP114- | TP114- | − | − | − | − | Accessory |
| 047 | 047 | | | | | |
| TP114- | TP114- | − | − | − | − | Accessory |
| 048 | 048 | | | | | |
| TP114- | TP114- | − | − | − | − | Core |
| 049 | 049 | | | | | |
| TP114- | ycfB | ++ | ++ | ++ | ++ | Core |
| 050 | | | | | | |
| TP114- | ycfA | ++ | ++ | ++ | ++ | Core |
| 051 | | | | | | |
| TP114- | yceB | − | − | − | − | Core |
| 052 | | | | | | |
| TP114- | hicB | − | − | − | − | Accessory |
| 053 | | | | | | |
| TP114- | TP114- | − | − | − | − | Accessory |
| 054 | 054 | | | | | |
| TP114- | TP114- | − | − | − | − | Core |
| 055 | 055 | | | | | |
| TP114- | yajC | − | − | − | − | Accessory |
| 056 | | | | | | |
| TP114- | TP114- | − | − | − | − | Core |
| 057 | 057 | | | | | |
| TP114- | yajA | − | ++ | ++ | ++ | Accessory |
| 058 | | | | | | |
| TP114- | yaiB | − | − | ++ | ++ | Accessory |
| 059 | | | | | | |
| TP114- | TP114- | − | ++ | ++ | ++ | Core |
| 060 | 060 | | | | | |
| TP114- | yaiA | − | − | − | − | Accessory |
| 061 | | | | | | |
| TP114- | yahA | − | − | − | − | Accessory |
| 062 | | | | | | |
| TP114- | TP114- | − | − | − | − | Soft-core |
| 063 | 063 | | | | | |
| TP114- | TP114- | − | − | − | − | Core |
| 064 | 064 | | | | | |
| TP114- | yagA | − | − | − | − | Core |
| 065 | | | | | | |
| TP114- | TP114- | − | − | − | − | Core |
| 066 | 066 | | | | | |
| TP114- | TP114- | − | − | − | − | Core |
| 067 | 067 | | | | | |
| TP114- | parA | ++ | ++ | ++ | ++ | Core |
| 068 | | | | | | |
| TP114- | yafA | − | ++ | ++ | ++ | Core |
| 069 | | | | | | |
| TP114- | yaeC | − | − | − | − | Core |
| 070 | | | | | | |
| TP114- | TP114- | − | − | − | − | Core |
| 071 | 071 | | | | | |
| TP114- | TP114- | − | − | − | − | Core |
| 072 | 072 | | | | | |
| TP114- | yadA | − | − | − | − | Accessory |
| 073 | | | | | | |
| TP114- | yadB | − | − | − | − | Accessory |
| 074 | | | | | | |
| TP114- | tnpA | − | − | − | − | Accessory |
| 075 | | | | | | |
| TP114- | aph-III | − | − | − | − | Accessory |
| 076 | | | | | | |
| TP114- | TP114- | − | − | − | − | Accessory |
| 077 | 077 | | | | | |
| TP114- | TP114- | − | − | − | − | Accessory |
| 078 | 078 | | | | | |
| TP114- | TP114- | − | − | − | − | Accessory |
| 079 | 079 | | | | | |
| TP114- | TP114- | − | − | − | − | Core |
| 080 | 080 | | | | | |
| TP114- | TP114- | − | − | − | − | Core |
| 081 | 081 | | | | | |
| TP114- | TP114- | ++ | ++ | ++ | ++ | Core |
| 082 | 082 | | | | | |
| TP114- | repA | ++ | ++ | ++ | ++ | Core |
| 083 | | | | | | |
| TP114- | TP114- | − | − | − | − | Core |
| 084 | 084 | | | | | |
| TP114- | yheC | − | − | + | − | Core |
| 085 | | | | | | |
| TP114- | yheB | − | − | ++ | ++ | Core |
| 086 | | | | | | |
| TP114- | ydhA | − | − | ++ | ++ | Core |
| 087 | | | | | | |
| TP114- | TP114- | − | − | + | − | Accessory |
| 088 | 088 | | | | | |
| TP114- | yhcA | − | − | + | − | Core |
| 089 | | | | | | |
| TP114- | TP114- | − | − | − | + | Core |
| 090 | 090 | | | | | |
| TP114- | yhbB | − | − | − | + | Soft-core |
| 091 | | | | | | |
| TP114- | TP114- | − | − | + | − | Soft-core |
| 092 | 092 | | | | | |

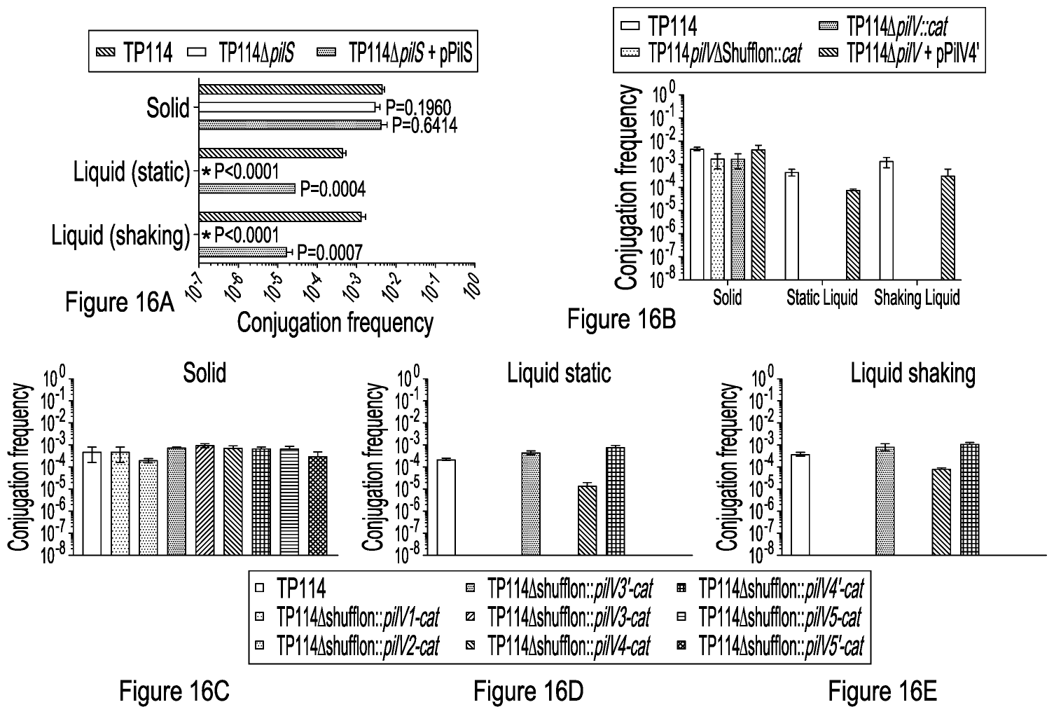

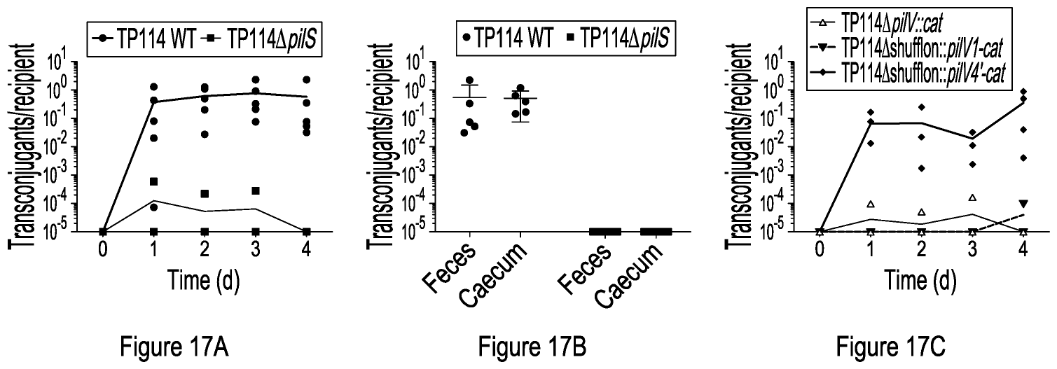

Confirmation of the TP114 mating pair stabilization module's essentiality for in vivo conjugation. The role of the T4P (pil genes) in mating pair stabilization in vitro was suspected for R721, another model plasmid of the IncI2 family. However, TP114 HDTM data suggested that the genes predicted to be involved in the formation of the T4P were essential for conjugation in vivo. This meant that the T4P was not required in vitro for solid mating and that the differences in environmental conditions made the T4P essential in vivo. To confirm this hypothesis, a complete abolition of the T4P functions would be desirable. The prepilin gene pilS was first selected for deletion as the HDTM data revealed a strong dependence on this gene for in vivo conjugation, and because of its crucial role in the structure of the T4P. The prepilin is a major subunit forming the T4P, it is first processed into pilin by specific peptidase and then secreted and assembled into a pilus. Deleting pilS is thus expected to abolish the formation of the pilus and prevent its assembly. The pilS gene was deleted from TP114 using a recombination approach. The resulting TP114ΔpilS::cat was then transferred by solid mating conjugation in KN01 for further testing. As such, TP114ΔpilS::cat was tested for its ability to conjugate from KN01 to KN03 in solid, liquid, and agitating liquid conditions (FIG. 16.A). Whereas the ability of TP114ΔpilS::cat to transfer during solid mating remained unchanged, a drastic drop in transfer efficiency was observed in the presence or absence of shaking in liquid. This suggests that conjugation efficiency of TP114ΔpilS::cat was impaired under unstable conditions compared to wild type TP114. Complementation using plasmid pPilS expressing pilS in trans resulted in significant recovery of conjugation rates. To test the involvement of the T4P for conjugation in the intestinal tract, a conjugation experiment was performed using a mouse model. A total of 5 Sm-treated mice were first fed with KN03 as a recipient strain and then with either KN01+TP114 or TP114ΔpilS::cat as donors. Conjugation efficiency was monitored through feces collection for four consecutive days (FIG. 17.A). On the fourth day, mice were sacrificed to compare the conjugation efficiency found in the feces and in the caecum (FIG. 17.B). While wild type TP114 was able to conjugate at its expected frequency (>10⁻¹), TP114ΔpilS::cat was unable to transfer in vivo. This reveals the crucial role of TP114's T4P for in vivo conjugation, and potentially explains the conservation of T4P across IncI2 conjugative plasmids.

Role of the pilV adhesin variants and the shufflon in TP114. Although the HDTM data indicated that only the N-terminus portion of pilV was essential for in vivo conjugation, it was suspected that this was in fact an artifact of the HDTM method. This anomaly is due to the presence of a shufflon at the C-terminus of pilV that re-organize the end of the gene to produce several variants. The pilV gene encodes an adhesin thought to be responsible for the recognition of the recipient cell by a donor bacterium. The adhesin is believed to be present on the tip of the T4P to establish contact and stabilize the interaction between the two cells. The shufflase was thus deleted to lock the shufflon in a stable conformation in TP114Δrci. Then, to assess the role of pilV and the shufflon for in vivo conjugation, the entire pilV gene was deleted in a first experiment, generating TP114ΔpilV-rci. In another variant, only the shufflon was replaced by a Flag-tag, generating TP114pilVΔshufflon-rci::cat. In yet another series of variants, the pilV gene variants were locked in a specific conformation (TP114pilVΔshufflon::pilV1-cat, TP114pilVΔshufflon::pilV2-cat, TP114pilVΔshufflon::pilV3-cat, TP114pilVΔshufflon::pilV3′-cat, TP114pilVΔshufflon::pilV4-cat, TP114pilVΔshufflon::pilV4′-cat, TP114pilVΔshufflon::pilV5-cat, TP114pilVΔshufflon::pilV5′-cat. This was accomplished to test if the C-terminus portion of pilV was essential for PilV-mediated adhesion, and to elucidate the importance of the different pilV variants. As such, TP114ΔpilV-rci, TP114pilVΔshufflon-rci::cat and all locked pilV variants were transferred to KN01 to test their role in mating pair stabilization. Each construct was transferred to KN03 recipient bacteria by conjugation under solid, liquid static and agitating liquid conditions. The pilV adhesin was found to be essential for conjugation in both liquid conditions, confirming the role of the adhesin in mating pair stabilization (FIG. 16.B). More precisely, the ability of TP114pilVΔshufflon-rci::cat to conjugate on a solid support was slightly decreased as compared to the control, but the ability to conjugate in liquid (shaking or not) was completely abolished (FIG. 16.B). This showed that the C-terminus portion of pilV was essential to generate functional adhesins, contrary to what was suggested by the HDTM data. Furthermore, transfer of TP114 pilV variants between two E. coli cells resulted in high conjugation efficiencies for all PilV configurations on solid medium (FIG. 16.C) while only some variants (namely TP114pilVΔshufflon::pilV3′-cat, TP114pilVΔshufflon::pilV4-cat, and TP114pilVΔshufflon::pilV4′-cat) were proficient for mating pair stabilization and conjugation understatic liquid (FIG. 16.D) and agitating liquid conditions (FIG. 16.E). This indicated that pilV variants recognized different structures at the surface of bacteria, and that these interactions were needed for plasmid transfer. As such, variantsTP114pilVΔshufflon::pilV3′-cat, TP114pilVΔshufflon::piV4-cat, and TP114pilVΔshufflon::pilV4′-cat recognized structures present at the surface of EcN, most likely lipopolysaccharides, while other variants recognized structures not found on EcN. To confirm that this interaction is required for conjugation in vivo, the pilV deletion mutant TP114ΔpilV-rci, one variant recognizing EcN (TP114pilVΔshufflon::pilV4′-cat) or not (TP114pilVΔshufflon::pilV1-cat) was used for an in vivo conjugation assay using KN03 as a recipient (FIG. 17.C). In this assay, only TP114pilVΔshufflon::pilV4′-cat was able to conjugate in vivo, confirming that the binding of a PilV adhesin to the surface of a recipient bacterium is needed for in vivo conjugation.

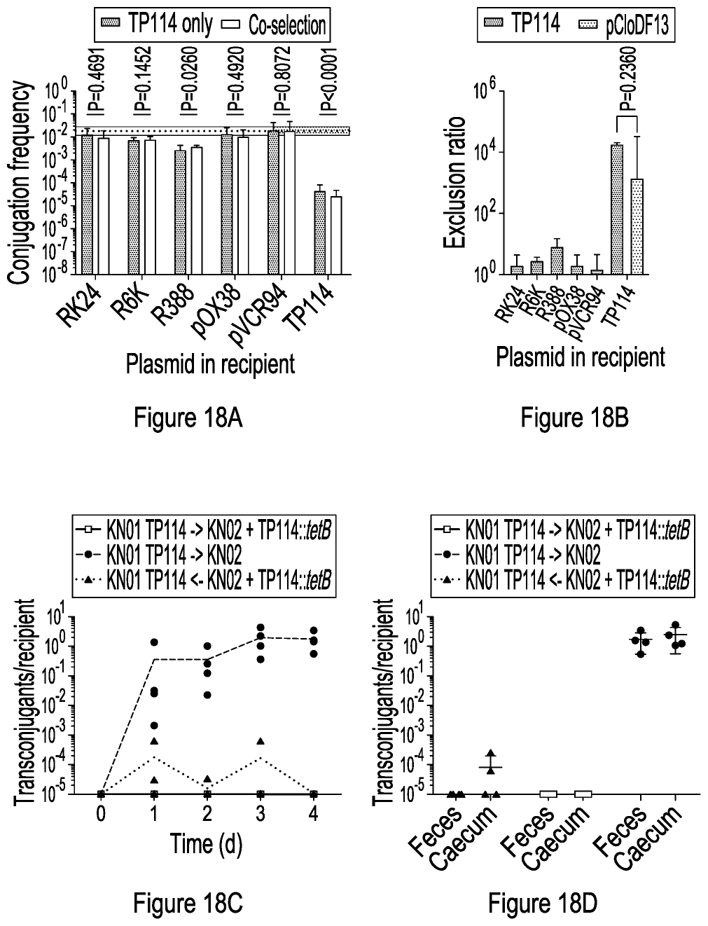

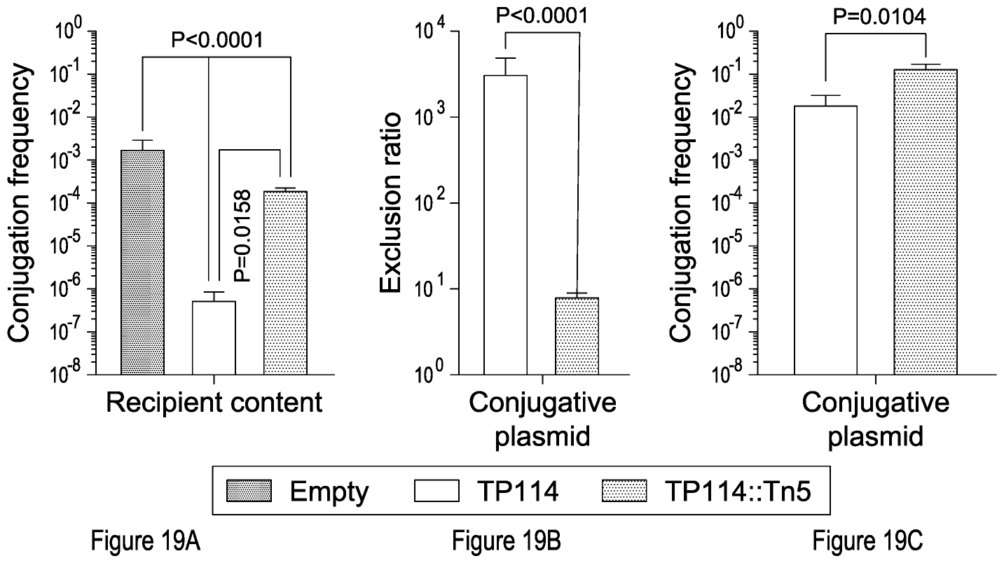

Identification of an exclusion protein of TP114. Exclusion and incompatibility are two mechanisms that prevent two plasmids to share a same cell. Whereas incompatibility is passive and occurs when two replication or maintenance system are too similar, exclusion is an active mechanism that prevent either cell-cell contact or DNA entry in the recipient. The existence and extent of exclusion in TP114 was first verified. To do so, a conjugation experiment from KN02 to KN01 bearing six different conjugative plasmids was carried. The selected plasmids spanned six incompatibility families and those families were first categorized based on incompatibility and exclusion, only TP114 should be able to mediate its own exclusion. As expected, the exclusion phenomenon was only observed with TP114 transferring to a TP114 bearing recipient (FIG. 18.A) (FIG. 18.B). However, in this experiment, it was impossible to discern incompatibility from maintenance and exclusion. This was addressed using a mobilizable plasmid pCloDF13 capable of hijacking TP114's T4SS to conjugate, but which uses a completely different replication mechanism. As shown in FIG. 18.B, the exclusion ratio of both TP114 and pCloDF13 were similar which proved that TP114 is subjected to exclusion. In a last effort to characterize this resistance mechanism, an exclusion experiment was carried in vivo where KN01+TP114 and KN02+TP114::tetB were used as conjugation pairs. Exclusion was found to be even more stringent in this environment, barely even producing transconjugants over a period of 4 days (FIG. 18.C). Of note, results were consistent between the feces and the caecum (FIG. 18.D). Using the HDTM library 2, the gene responsible for exclusion was investigated. In the final step of HDTM, TP114::tetB was transferred to the HDTM library 2, and transconjugants were sequenced. Most of the transconjugants had transposon insertions in TP114-005, a gene of previously unknown function (Table 12). To confirm that the exclusion protein was TP114-005, clones from the HDTM library 8 were transferred first to MG1655Nx^{R}, and then back into KN02. The use of two successive rounds of transfers should isolate the TP114::Tn5 clone since in vitro conjugation efficiencies allows TP114 to transfer to 1% of the cells, hereby reducing the transfer to a same recipient cell to a minimum. Also, even with deficient exclusion, incompatibility should prevent the persistence of two different clones of TP114 within a same cell through maintenance incompatibility mechanisms. As insertions in TP114-005 are more frequent, they should also be easily isolated. Exclusion capacity of the three clones of TP114::Tn5 was tested by transferring TP114::tetB from KN01 into KN02. Most of the clones were incapable of exclusion as shown by the high conjugative frequencies (FIG. 19.A) and exclusion ratio (FIG. 19.B). Surprisingly, while testing the ability of these clones to conjugate, exclusion deficient clones were nearly 10-fold more efficient than wild type TP114 (FIG. 19.C). Therefore, it is understood that the TP114-005 gene and the corresponding protein were involved in the exclusion process.

TABLE 12
Over-representation of some genes
in the exclusion HDTM library 8.

| | Replicate | | |
|---|---|---|---|
| Gene | 1 | 2 | 3 |
| TP114-005 | 70.6% | 77.6% | 76.9% |
| ydjA | 1.0% | 0.7% | 0.8% |
| yaeC | 7.7% | 3.8% | 8.9% |
| pilQ | 0.7% | 0.9% | 1.0% |
| ydgA | | 0.7% | 0.6% |
| yaiA | 1.3% | 1.0% | |
| yadA | | 0.7% | 0.5% |
| TP114-080 | 0.7% | 0.7% | |
| TP114-048 | 1.6% | 1.5% | |
| pilR | | 1.5% | 1.5% |
| yebA | | | 0.8% |
| TP114-045 | | | 1.2% |
| pilN | | | 0.9% |
| pilM | 1.2% | | |
| nikB | 0.8% | | |
| hicB | 0.7% | | |

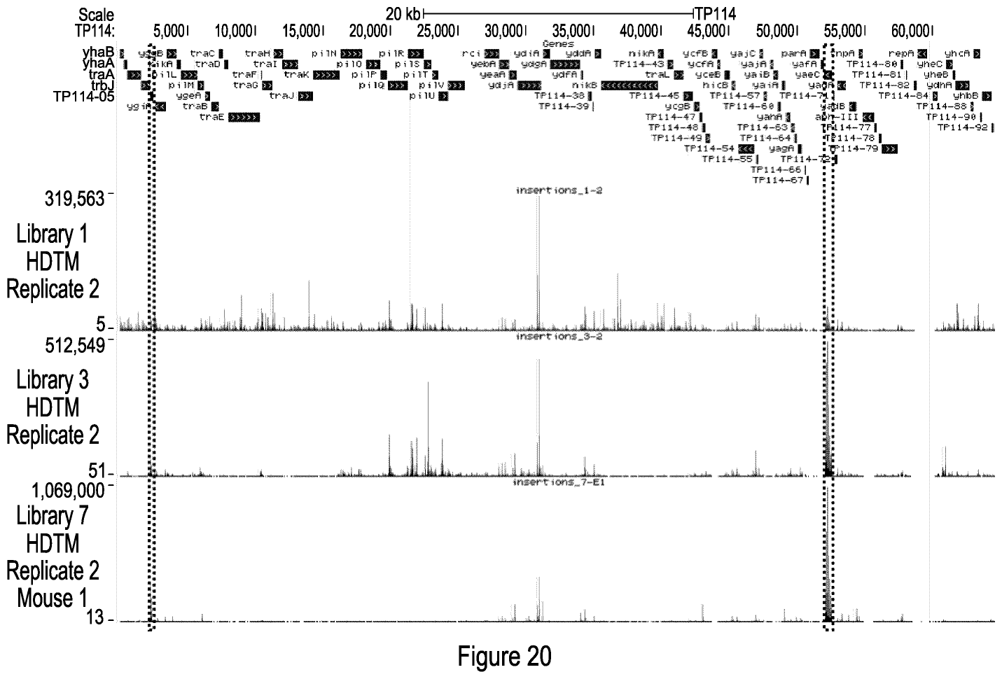

Identification of genes with detrimental effects on TP114 conjugation. The HDTM experiment helped to discover genes which limit TP114 conjugation transfer frequency. These genes can be determined by looking at genes having a count ratio that increased following each transfer steps. In our experiments, only two genes were found to be detrimental to TP114's transfer both in vitro and in vivo. These genes were TP114-005 (which was previously identified as the exclusion protein) and yaeC (TP114-070) (FIG. 20). Interestingly, gene function analysis revealed that yaeC is a homolog to finO, a transcription factor that represses genes in the IncF family of conjugative plasmid. The yaeC gene is therefore most likely a transcription repressor that limits TP114's transfer, both in vitro and in vivo. The deletion of this gene could be desirable to enhance TP114 transfer both in vitro and in vivo.

### EXAMPLE III—CONSTRUCTION OF CONJUGATIVE DELIVERY SYSTEMS DERIVED FROM TP114

Strains, plasmids and growth conditions. All strains and plasmids used in this example are described in Table 1. All plasmid sequences are provided in the sequence appendix. Oligonucleotides used in this example are listed in Table 2. Details on strain growth conditions, DNA manipulation, plasmid construction, recombineering and routine transformation can be found in the Material and Method section of the Example I.

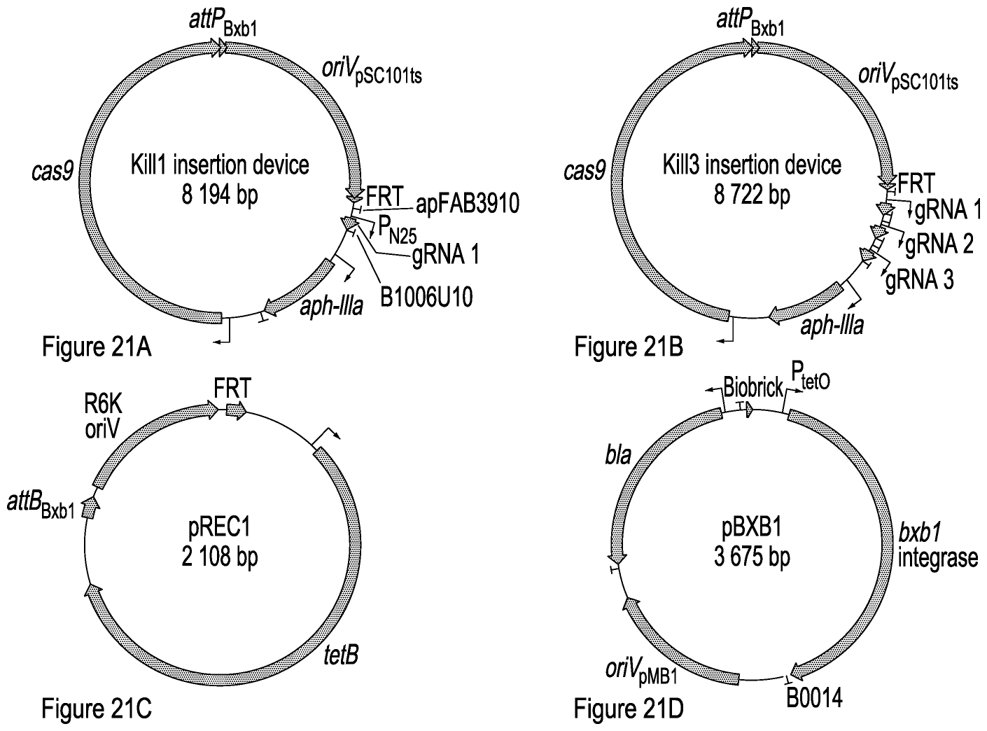

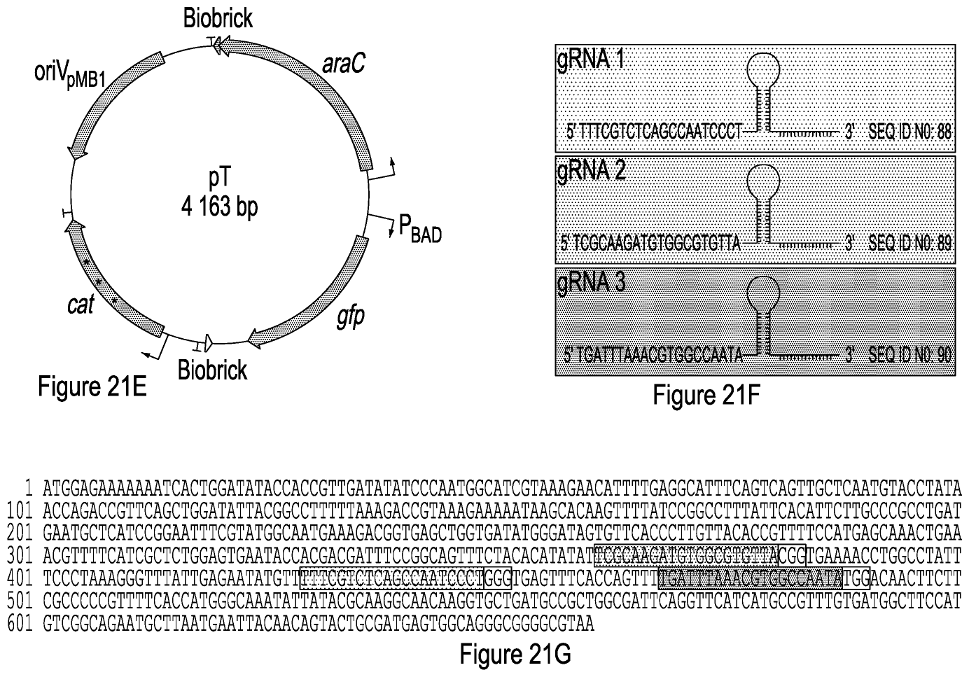

Construction of the loading dock pREC1. pREC1 is a plasmid used as a template to amplify a loading dock cassette and to insert it into the transfer machinery in one simple step. In this example, pREC1 was used to insert a loading dock into the TP114 transfer machinery. To do so, pREC1 was first assembled into a plasmid by Gibson assembly (amplifying the tetB resistance gene from pFG018 and the oriV_{R6K} from pKD4). An FRT and attP_{BXb1} sites were provided by the primer assembly tails. The resulting plasmid pREC1 (FIG. 21.C) was then used as a template for subsequent PCR amplification. The FRT-tetB-attP_{Bxb1} cassette was inserted in TP114 by recombineering, creating TP114::tetB plasmid (Datsenko et al., 2000). This version of TP114 can then be used for the insertion of the genetic cargo by Double Recombinase Operated Insertion of DNA (DROID).

Construction of pBXB1. The plasmid pBXB1 contains the integrase Bxb1 and was assembled by amplifying the oriV_{pMB1} and the ampicillin resistance gene (bla) from pSB1A3, and the Bxb1 integrase gene from gBlock-Bxbl (FIG. 21.D). The promoter of the Bxb1 integrase allows constitutive expression of the Bxb1 integrase. The two PCR products were purified by SPRI and pBXB1 was constructed by Gibson assembly before transformation in chemically competent EC100Dpir+.

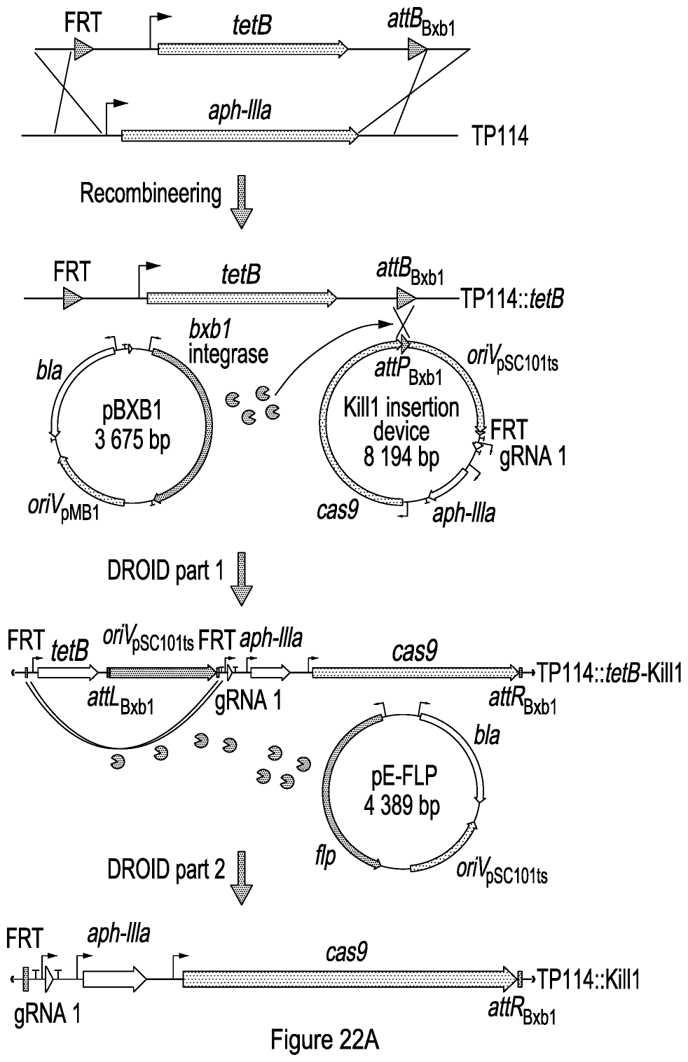

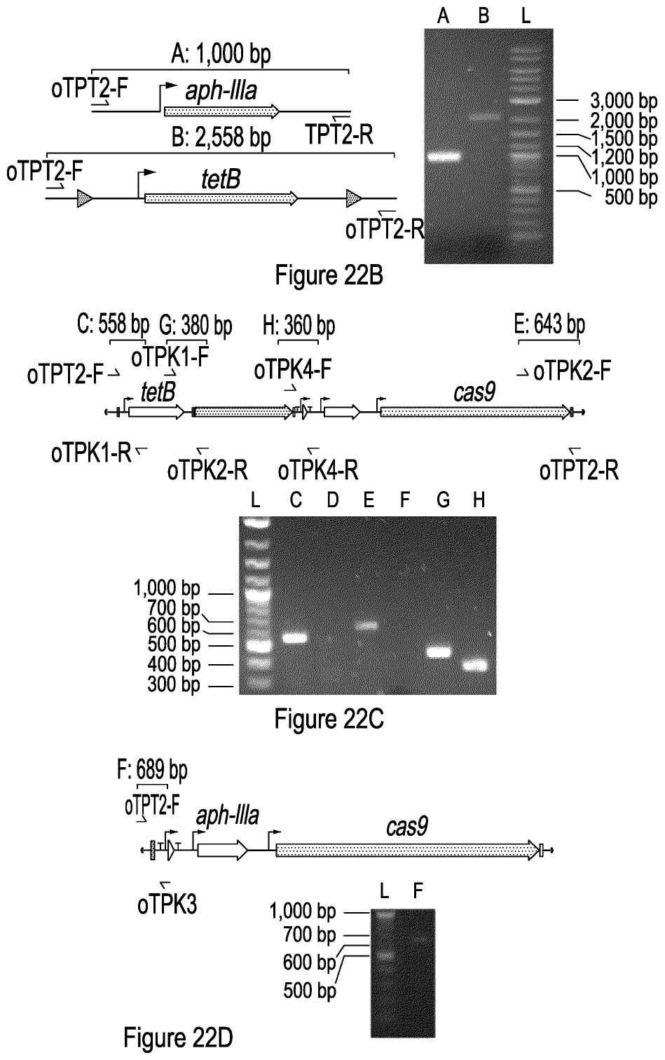

Double Recombinase Operated Insertion DNA (DROID). To combine on a same vector the genetic cargo and the transfer machinery, a new process hereby termed DROID, was developed (FIG. 22.A). In the present example, the DROID method was used to combine two DNA molecules together without destabilizing the plasmid replication. The DROID method requires both DNA molecules to contain specific integration and recombination sites. For the transfer machinery, the recombination sites are provided by the loading dock present in the tetB insert of TP114::tetB (FIG. 22.B). For the genetic cargo, the recombination sites are provided during its assembly in a temporary backbone termed «insertion device» (FIG. 21.AB), which is removed during the DROID procedure. Both pBXB1 and one of the genetic cargo insertion devices were transformed in the same EC100Dpir+ strain by electroporation. The modified TP114::tetB was then transferred by conjugation at 30° C. into EC100Dpir+ containing pBXB1 and one of the Kill1 or 3 insertion devices. Since the integrase is constitutively expressed, a single subculturing step is enough to promote integration. The resulting plasmid TP114::tetB-Kill1 or 3 was then transferred by conjugation to E. coli MG1655Nx^{R}. Transconjugants bearing both the selection module from the genetic cargo and from TP114::tetB were selected. From this point forward, the strains were cultivated at 37° C. to avoid activation of the thermosensitive origin of replication (oriV_{pSC101ts}) from the integrated genetic cargo that could destabilize TP114. E. coli MG1655Nx^{R}+TP114::tetB-Kill1 or 3 were then transformed with pCP20 by electroporation. pCP20 expresses the FLP recombinase which recognizes both the FRT site in the recombineering cassette and the FRT site in the genetic cargo, and recombines them together to knock out tetB, the attL_{bxb1} site, and the oriV_{pSC101ts} which constitutes the insertion device (FIG. 22.A). Integrity of the inserts was assessed by PCR for TP114, TP114::tetB (FIG. 22.B), TP114::tetB-Kill1 (FIG. 22.C) and TP114::Kill1 (FIG. 22.D) (data not shown for TP114::Kill3). The DROID technique could also rapidly be adapted for several applications such as chromosomal insertion of large constructs and assembly of very large plasmids or exogenous chromosomes.

Construction of the Cas9 test genetic cargo insertion devices. Two genetic cargos coding for cas9 and gRNA(s) were inserted in the transfer machinery using the DROID method. While assembly of such a large cluster of genes is usually complex and requires multiple steps, the DROID approach considerably simplifies the process. One of the most important aspects of the cas9-gRNA gene cluster is to design highly specific gRNAs. To design such gRNAs, DNA 2.0 (ATUM) web-based software was run with the chloramphenicol acetyl-transferase gene (cat) as the target sequence and E. coli K-12's genome as the off-target sequence. The most potent gRNA spacers (highest AG, lowest off-targets) were then run into BLASTn (Altschul et al., 1990) against Enterobacteriaceae and EcN genomes to eliminate any candidate gRNAs with high off-targeting. This bioinformatic strategy identified three gRNAs (namely gRNA 1, 2 and 3) (FIG. 21.FG). The genetic cargo was introduced into the transfer machinery vector, in a two-step approach. To do so, a first plasmid was created by combining the cas9 gene from pKN02, the kanamycin resistance cassette from pKD4, and the oriV_{pSC101ts} from pGRG36 using corresponding primers (Table 2). gRNA 1 was amplified entirely from a previous construct (pKN02), and the gRNA 2 and 3 spacers were added directly in PCR primers. A first genetic cargo containing the cas9 gene with gRNA 1 (Kill1) was generated. Another genetic cargo encoding the cas9 gene with gRNA 1, 2 and 3 (Kill3) was also obtained. For genetic cargo Kill3, assembly tags were placed between each gRNAs to prevent miss-assembly due to the repetitive nature of the gene locus. Other fragment junctions facilitated the addition of short sequences in the primer tail such as an FRT site, between gRNA 1 and the oriV_{pSC101ts}, and the attP_{bxb1} site, between oriV_{pSC101ts} and cas9. Both genetic cargo insertion devices were constructed by Gibson assembly. All gRNA sequences were confirmed by Sanger sequencing (data not shown). Complete plasmid maps of Kill1 and Kill3 are shown in FIGS. 21.A and 21.B respectively.

In vitro conjugation assay. In vitro conjugation experiments were performed as described in the example I Material and method section.

Generation of chloramphenicol resistant Citrobacter rodentium. Details about the use of pGRG36 and Tn7 mediated insertion of DNA is provided in the material and method section of Example I and in FIG. 1. Specifically, integration plasmid pGRG36-SmCm was transformed in C. rodentium DBS100 by electroporation. To mediate cassette insertion into glmS gene's terminator, C. rodentium DBS100 was first cultivated at 30° C. in LB with arabinose until OD₆₀₀ₙₘ 0.6. Then, cells were heat-shocked at 42° C. for 1 hour and incubated at 37° C. overnight to allow for plasmid clearance. Cells were then streaked onto a LB agar plate selecting only the insert. Plasmid curation and insertion of the cassette in the genome was confirmed using the primers oGSC6-F, oGSC6-R, oGSC5-F, opir3-F and opir3-R from Table 2.

Construction and test of pNA22, pNA23 and pNA24. The pNA22 to 24 plasmid suite was designed to delimit the minimal DNA sequence responsible for the replication of TP114. Replication initiation sequences are usually located near the repA gene previously identified in TP114 as TP114-083 (see Example II) (Praskier et al., 2005). In order to isolate the minimal DNA sequence for replication, repA and a 1,000-bp region located either upstream or downstream of repA, or both, were cloned into pKD3 by Gibson assembly to yield pNA22 to pNA24. Since pKD3's replication is pir-dependent, the constructions were transformed into chemically competent E. coli EC100Dpir+(Metcalf et al., 1994). Then, functionality of the replication origin from TP114 was tested by transformation in E. coli BW25113.

Generation of pir+EcN (KN05) strain. The pir gene was amplified from EC100Dpir+ and assembled with rrnB terminator from pFG018 and a SmaI+XhoI digested pGRG36 backbone. The resulting plasmid pGRG-pir+ (Kvitko et al., 2012) was then transformed in MFDpir+ (Ferrières et al., 2010) and mobilized towards EcN by RP4 mediated conjugation. To induce pir insertion into glmS gene's terminator, EcN was first cultivated at 30° C. in LB with arabinose until 0.6 OD₆₀₀ₙₘ. Then, cells were heat-shocked at 42° C. for 1 hour and incubated at 37° C. overnight to allow for plasmid clearance. Plasmid curation was tested by streaking 20 colonies on plates with or without ampicillin to select for pGRG36's backbone. The clones for which plasmid curation was confirmed were then screened by PCR for pir insertion using the respective primers presented in Table 2.

Host constrained replication of the transfer machinery. A cassette coding for cat and oriV_{R6K} was amplified from pKD3 and used to replace TP114-083's CDS (repA) in an E. coli EC100Dpir+ strain. Recombinant clones were screened by PCR using the corresponding primers from Table 2. Replication of the resulting plasmid TP114ΔrepA::cat-oriV_{R6K} should be dependent on a pir gene located in E. coli EC100Dpir+'s chromosome. pir-dependent replication of TP114ΔrepA::cat-oriV_{R6K} was verified by conjugative transfer in pir+ (KN05 and EC100Dpir+) or pir− hosts (KN01).

In silico oriT_{TP114} prediction. The origin of transfer (ori7) is usually located near the promoter of the nickase in conjugative and mobilizable plasmids. In TP114, a large intergenic region is located near the promoter of nikAB (TP114-041 and TP114-042) that encodes the predicted nickase proteins. In silico analysis of the potential oriT of TP114 (oriT_{TP114}) sequence was first compared to other IncI2 plasmids listed in Table 5 using BLASTn to find highly conserved regions in the potential oriT_{TP114}. This allowed to narrow down oriT_{TP114} to a 138-bp core sequence. This core oriT_{TP114} was then compared to the minimal oriT_{R64} (Furuya and Komano, 2000) by Pairwise Sequence Alignment using EMBOSS Needle web-based software (Rice et al., 2000). The alignment was performed using default settings (a cost of 10 for gaps creation, 0.5 for extension). Results were then manually annotated to indicate the positions of important repeats, and putative binding and nicking sites.

Construction of pNA01 and derivatives. The whole intergenic region comprising the predicted oriT_{TR114} was amplified, cloned, and assembled with the broad host-range oriV_{pBBR1} from pSIM7, and tetB from pFG018, using Gibson assembly hereby creating pNA01. An alternative plasmid (pNA02) contained a 7-bp deletion centered on the predicted nicking site of oriT_{TP114} sequence and was assembled with the same backbone as pNA01. A kanamycin resistant variant of pNA01 and pNA02 (pKN30 and pKN31 respectively) was generated by amplifying the backbone of pNA01, or pNA02, and by amplifying the kanamycin resistance gene from pKD4. PCR fragments were then purified and assembled together using Gibson assembly.

Deletion of oriT_{TP114}. An FRT-flanked cat cassette was amplified from pKD3 to delete, by recombineering, a portion of the predicted oriT_{TP114} comprising the nicking site. Deletion clones were verified by PCR and were then used in conjugation experiments to assess the impact of the deletion on transfer frequency.

Statistical analysis. Statistical significance was performed as described in the material and method section of the Example I.

3.0—Conformations of the Conjugative Delivery System

Variations in the genetic cargo's delivery mode. The genetic cargo nucleic acid molecule can be delivered using different approaches, each with their advantages and potential inconvenients. In this section, COP delivery modes will be explored. First, the COP can be decomposed in several component as shown in FIG. 23.A. The COP is first composed of a probiotic bacterium as well as a conjugative delivery system. The conjugative delivery system is itself composed of the transfer machinery and the genetic cargo which themselves contains functional gene modules. As such, the conjugative delivery system can be decomposed into several vectors and can be partially integrated in the chromosome of the probiotic donor. A first strategy to mobilize the genetic cargo is to insert it into the transfer machinery to form a single vector which can autonomously propagate in a population of bacterial cells. While this method can be desirable for applications where maximal conjugal transfer is required, it can also lead to persistence of the conjugative delivery system in the microbiota. Therefore, alternative methods of propagation that provide a transient expression of the genetic cargo should also be considered. For example, it can be necessary for the genetic cargo to be expressed at different levels through time or for a short period of time (e.g. expression of insulin stimulating peptides like GLP-1). Alternatively, if the genetic cargo mediates a negative effect on a bacterial population, its persistence in the environment could lead to the emergence of resistant bacteria. Examples of such alternative method includes constrained cis mobilization and in trans mobilization which are presented in FIG. 23.B and are discussed in the next sub-sections.

3.1—Genetic Cargo Delivery by Cis Mobilization

Cis mobilization as a potent conjugative delivery system mode. Cis mobilization is a delivery mode in which both the transfer machinery and the genetic cargo are found on the same vector. In this setup, the Conjugative Delivery System is transferred to the recipient bacterium, which in turn becomes a new donor. Through this process, the conjugative delivery system and the genetic cargo are transferred at an exponential rate, since one donor bacterium can create multiple donor cells and trigger a chain reaction. Due to this exponential diffusion, cis mobilization is theoretically the most efficient delivery mode to propagate the genetic cargo within a bacteria population, but it also leads to conjugative delivery system persistence in the environment. One of the main challenges of the cis-mobilization strategy is to link the Transfer machinery and the genetic cargo on a same vector.

DROID is a potent method for the fusion of DNA molecules. Several methods can be employed to mediate the association of two DNA molecules, for example Gibson assembly, digestion-ligation, Golden Gate, USER-cloning and other derivatives. One alternative is to use recombination-based techniques such as Recombineering, Gene doctoring, or NO-SCAR. However, although those techniques can easily delete large DNA fragments, they are limited by the length of the DNA molecule that can be inserted into a specific location. The DROID method presents several advantages over existing methods as it can be done very quickly and its use of the serine-integrases makes it highly specific to orthogonal insertion sites. DROID allows easy insertion of large DNA molecule that carry complex gene clusters since the fusion of both DNA molecules together is independent of their size. After the fusion of the DNA molecules, undesirable DNA sequences can be easily removed through the action of a second recombinase that excises specific DNA fragments. The DROID method leaves signature scars at either end of the insertion site (one FRT site and one attR_{Bxb1} site) which are not homologous to one another. This prevents recombination between scars that could knock-out the inserted DNA molecule.

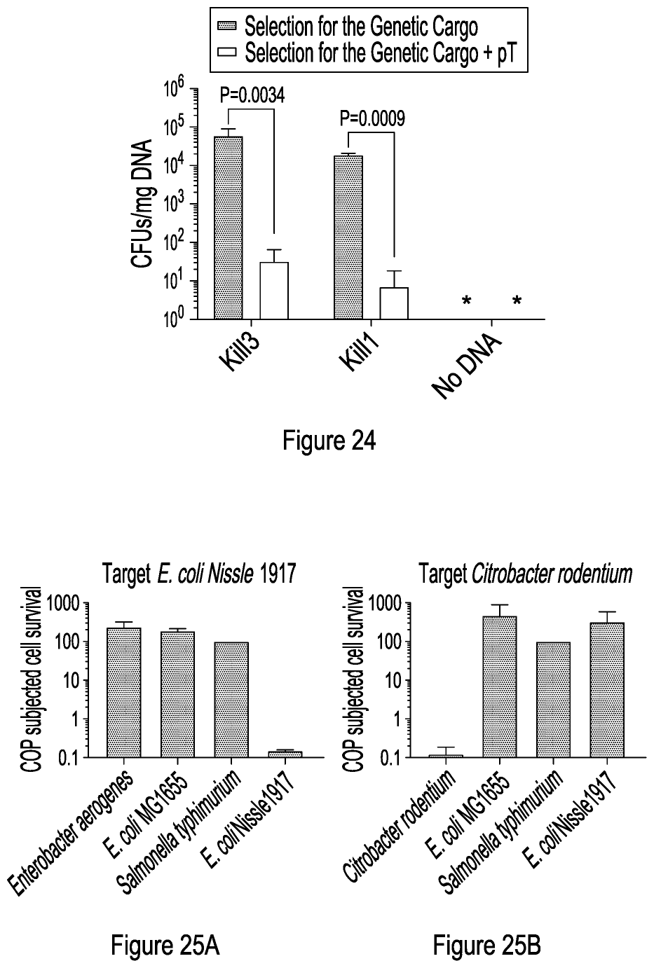

Functional test of the genetic cargo insertion devices. Two genetic cargo insertion devices were constructed to test the DROID method. Since in this example the genetic cargo is composed of a cas9-gRNA complex targeting the cat gene, a regular transformation of Kill1 or Kill3 insertion devices, in a cell containing a plasmid bearing the cat gene (pT) should lead to plasmid loss. Therefore, as a control to validate that the genetic cargos were functional, the genetic cargo insertion devices were first directly transformed in a cell containing the pT plasmid. Transformation efficiency was found to be significantly higher when selecting only for the genetic cargo insertion devices instead of both the insertion device and the pT plasmid simultaneously (FIG. 24). Furthermore, since pT codes for GFP (FIG. 21.F), its presence in the cell produces a fluorescent signal that can easily be visualized under blue light. The CFUs from the transformation of both Kill1 and Kill3 insertion devices were all GFP negative, showing clearance of pT within the transformants. This indicated that both genetic cargos were functional in their insertion devices.

Insertion of two test genetic cargos in the transfer machinery by DROID. The DROID method can be divided in three simple steps (FIG. 22.A). The first step is to insert the loading dock into the target DNA molecule by recombineering (FIG. 22.B). This step is only required once. The resulting plasmid can then be used to insert several different genetic cargos using the same approach. In the present example, the loading dock is a fragment of pREC1 that encodes for FRT-tetB-attB_{Bxb1} and it was inserted in TP114, replacing the aph-Ill gene and generating TP114::tetB. The second step requires both the genetic cargo insertion device and the plasmid pBXB1 to be cloned in the same cell. Two genetic cargo insertion devices were used to test the DROID method: Kill1 (FIG. 21.A) and Kill3 (FIG. 21.B). TP114::tetB was transferred by conjugation towards E. coli EC100Dpir+ containing pBXB1 and Kill1 or 3. Then, TP114::tetB-Kill1 or 3 (FIG. 22.C) were transferred towards E. coli MG1655Nx^{R}. The last step of the method is to transform E. coli MG1655Nx^{R}+TP114::tetB-Kill1 or 3 with pCP20. This step enables the removal of tetB, attL_{BXb1} and oriV_{pSC101s} (FIG. 22.D). The final conjugative delivery systems, TP114::Kill1 and TP114::Kill3, were transferred into KN01 and KN01ΔdapA. The whole process from step 1 to 3 requires 11 days to complete, but step 1 is not required each time. This thus reduces the time to perform a clean insertion of the genetic cargo to 7 days.

Cis mobilization of genetic cargos in different cell types. To test whether the genetic cargos were functional after DROID into the transfer machinery (TP114), TP114::Kill1 was used as a model. The conjugative delivery system TP114::Kill1 was transferred into a 1:1 mix of four different recipient cells: KN02, E. coli MG1655Nx^{R}, Enterobacter aerogenes, and Salmonella typhimurium. In this mix, only KN02 is resistant to chloramphenicol and therefore, only KN02 should be targeted by the cas9-gRNA genetic cargo. The efficiency of TP114::Kill1 was evaluated through COP subjected cell survival ratios. These COP-subjected cell survival ratios represent the proportion of cells that received the conjugation delivery system and survived. The COP-subjected cell survival ratios were calculated by dividing the conjugation efficiency of the TP114::Kill1 (cells that survived the conjugative delivery system) by the conjugation efficiency of TP114 (total cells that should have received a conjugative delivery system). As expected, only KN02 was eliminated by the conjugative delivery system (FIG. 25.A). To confirm that the killing was not due to off-targeting in KN02's genome, a second mix of four recipients was prepared (Citrobacter rodentium KN04, E. coli MG1655Nx^{R}, KN03, Salmonella typhimurium). In this mix, only C. rodentium KN04 is resistant to chloramphenicol due to an insertion of the cat gene in its chromosome. The COP subjected cell survival ratios was again used to assess TP114::Kill1's efficiency. As predicted, only C. rodentium KN04 was affected by the COP system, thereby showing the cas9-gRNA genetic cargo could be expressed in different cells and was highly specific to the cat gene (FIG. 25.B).

The DROID method successfully fused the transfer machinery and the genetic cargo in a single vector. In the present example, it was shown that the DROID method could be used to join the transfer machinery with the genetic cargo in three steps. These steps were designed to avoid functional redundancy between the genetic cargo and the transfer machinery. The FRT recombination step was needed to eliminate the selection module from the transfer machinery and the maintenance module from the genetic cargo thereby creating a single DNA molecule with a single selection and vegetative replication module. The present conjugative delivery system was then used in a proof of concept of genetic cargo delivery towards several representatives of Enterobacteriaceae, a taxonomic group of bacteria that often are the cause of antibiotics resistant enteric and urinary infections.

3.2—Genetic Cargo Delivery by Constrained Cis-Mobilization

Constrained cis-mobilization as a biosafety measure. A constrained cis-mobilization system consists of a conjugative delivery system in which the genetic cargo and transfer machinery are located on the same DNA molecule. However, in such a system, the essential replication initiation gene from the maintenance module is inserted in the chromosome of the donor strain (FIG. 23.B). This makes replication of the conjugative delivery system dependent on the COP chromosome. Such a system would still be able to propagate at an exponential rate since the recipient strain would be able to express the genes from the conjugative delivery system. However, the conjugative delivery system will eventually be lost since the system cannot replicate in the recipient bacteria. Therefore, while the system exhibits high conjugative efficiency, it would not persist in the environment, thereby allowing for better control over administration dynamics and better biosafety level.

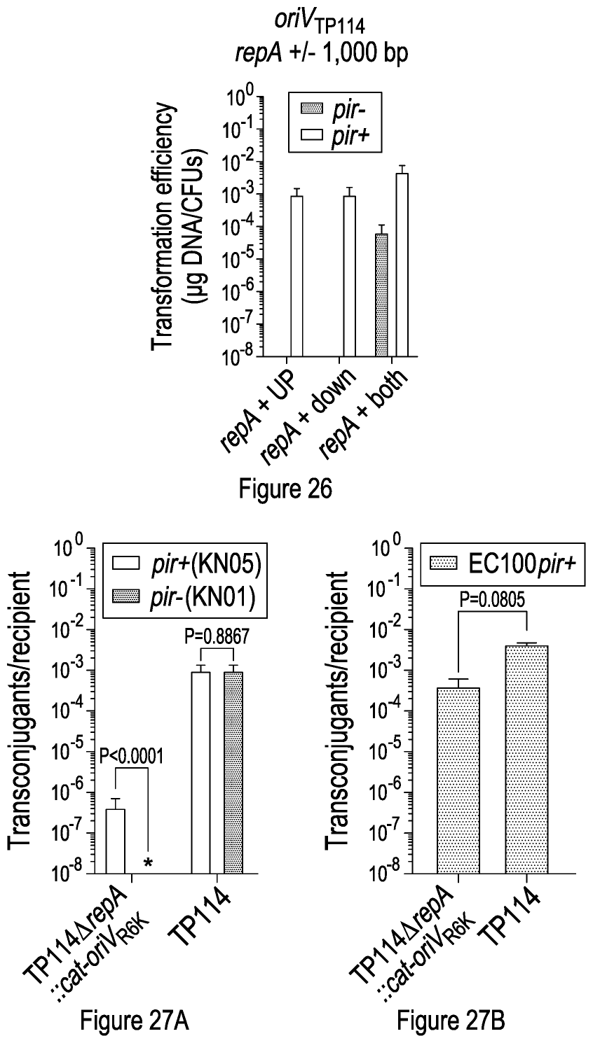

Localization of oriV_{TP114}. The first step toward constrained replication is to confirm the localization of the origin of replication of TP114 (oriV_{TP114}) since constrained cis-mobilization requires to relocate repA to the donor cell chromosome. Conjugative plasmid replication is usually initiated by a plasmid-encoded protein called RepA. This protein recruits the DNA replication machinery from the host cell and allows for the replication of plasmid DNA. The replication initiation protein encoded by TP114 was predicted by in silico analysis to be encoded by the repA gene TP114-083 (see example II). In most conjugative plasmids, the oriV can be found in an intergenic region near the repA gene. However, in TP114, repA is flanked by two intergenic regions. It was unclear as to which one is oriV_{TP114}. Consequently, repA was cloned with either 1,000-bp upstream, downstream or 1,000-bp on both sides of the gene into a plasmid backbone. The plasmid backbone contains oriV_{R6K}, an origin of replication that is dependent on a chromosomally integrated pir gene. Assemblies were first verified in a pir+ strain E. coli EC100Dpir+ and then cloned in a pir− strain BW25113 for oriV_{TP114} functionality analysis (FIG. 26). From the three plasmids tested, only one could replicate in a pir-host. This plasmid contained repA and 1,000 bp on both sides of the gene. This result indicated that TP114-083 encoded the actual RepA protein and that oriV_{TP114} is located near the repA gene.

Constrained cis mobilization of TP114. Constrained cis-mobilization refers to a conjugative delivery system in which the transfer machinery and the genetic cargo are located on a same vector that replicates under the control of a chromosomally encoded replication protein. In order to develop constrained cis-mobilization, a characterization of the replication protein and its associated oriV is required. While oriV_{TP114} and its repA gene were localized, several other factors have to be taken in consideration for the selection of the replication protein and oriV pair. For instance, the replication protein must be rarely found in the chromosome of potential recipient cells to avoid maintenance of the conjugative delivery system in the environment. Also, to reduce incompatibility based rejection of the conjugative delivery system, it can be desirable to use a replication system heterologous to the one of TP114. One of the most studied and understood replication system is R6K's pir-dependent oriV_{R6K}. The Pi protein, encoded by the pir gene, is naturally found in conjugative plasmids from the IncX family and present the advantage of not being found frequently in bacterial chromosomes. A pir integration system based on the Tn7 integration plasmid pGRG36 was developed and a pir+ EcN strain KN05 was generated. The TP114-083 (repA) was replaced by the oriV_{R6K} and a chloramphenicol resistance cassette (cat) using the commercially available E. coli EC100Dpir+ cloning strain generating TP114ΔrepA::cat-oriV_{R6K}. This plasmid was then transferred into KN05 and KN01 to evaluate the capacity of TP114ΔrepA::cat-oriV_{R6K} to replicate in a pir+ and pir− strain respectively. The conjugation efficiency was compared to wild-type TP114 under the same condition (FIG. 27.A). As expected, no transconjugants could be retrieved from the transfer of TP114ΔrepA::cat-oriV_{R6K} towards KN01 (pir− strain), but transconjugants were obtained for the conjugation towards KN05 (pir+strain). However, the conjugation efficiency of TP114ΔrepA::cat-oriV_{R6K} was lower than that of wild type TP114. A second conjugation experiment was attempted, this time using KN05 as the donor strain and E. coli EC100Dpir+ as the recipient (FIG. 27.B). The conjugation efficiency of both plasmids was then equivalent, thereby showing in this case that constrained cis-mobilization had very little impact on conjugation efficiency. Using such maintenance module, plasmid transfer towards pir− strain should remain unaffected, but replication should be impossible in the recipient cell. This means that expression of the genetic cargo's payload and the transfer machinery can still be achieved in pir− strains, but this expression is transient. Such transitory expression is desirable in several situations where constant expression can be detrimental to the COP effect, to the environment, or to the subject. Constrained cis-mobilization is therefore an efficient way to prevent plasmid persistence while still maintaining high conjugation efficiency.

3.3—Genetic Cargo Delivery by in Trans Mobilization

In trans mobilization as a delivery mode for the Genetic Cargo. In trans mobilization is achieved by using a vector system in which the transfer machinery and the genetic cargo are present on two different DNA molecules (FIG. 23.B). In this conformation, the genetic cargo must contain a transport module that is compatible with the transfer machinery's vegetative replication module. The transfer machinery can be immobilized either by insertion in the chromosome or disruption of its own transport module (oriT). This mode of delivery therefore allows the genetic cargo to be mobilized in trans (on a different DNA molecule independent from the transfer machinery) towards a recipient bacterium. The choice of vegetative replication module for the genetic cargo can have great importance over its persistence in the environment. This mode of delivery allows for maximal biocontainment, but more modest transfer efficiency as recipient bacteria cannot propagate the genetic cargo.

Localization of TP114's origin of transfer (ori7). All mobilizable plasmids are dependent over the recognition of their oriT by a relaxosome to enable the conjugation process to occur. The relaxosome is a multi-heteromeric protein complex that recognizes and binds to specific sequences on the oriT and cleaves a single strand of DNA at a specific location named the nicking site. The single strand of DNA is then guided to the T4SS, and transferred through the T4SS to the recipient cell. Mobilizable plasmids that do not encode a T4SS can use the T4SS encoded by another conjugative plasmid. However, mobilizable plasmids often encode their own relaxosome which are specific to their own oriT sequence. The oriT is therefore one of the most important sequence for genetic cargo transfer by the transfer machinery. Previous examples presented here exploited a physical link between the genetic cargo and the transfer machinery to use the oriT sequence from the transfer machinery in cis for DNA transfer. However, as observed with mobilizable plasmids, it is possible to relocate the oriT sequence from the transfer machinery to the genetic cargo nucleic acid molecule to only mobilize, in trans, the genetic cargo to the recipient cell. Importantly, oriT_{TP114} first needed to be identified. Based on other conjugative plasmids topology, the oriT sequence is often located near the nickase gene, a subunit of the relaxosome. On TP114, the nickase is predicted to be TP114-041 (nikB), a core gene that shares the same protein family pfam03432 domain as previously identified nickases. In TP114, a 368-bp intergenic region with two diverging genes is located near the promoter region of nikB and represented a potential oriT.

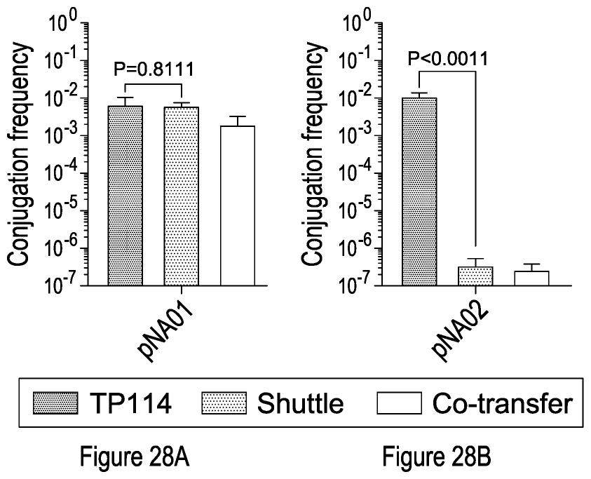

Construction of in trans-mobilizable vectors pNA01. The 368 bp predicted oriT_{TP114} was cloned into a broad-host-range plasmid backbone containing a vegetative replication module (oriV_{pBBR1}) and a payload module (tetM). Genetic cargo pNA01 was transformed in KN01ΔdapA containing TP114 and then mobilized towards KN02 (FIG. 28.A). Conjugation efficiency of pNA01 was comparable to that of TP114, confirming that the 368 bp DNA sequence cloned into pNA01 is oriT_{TP114}.

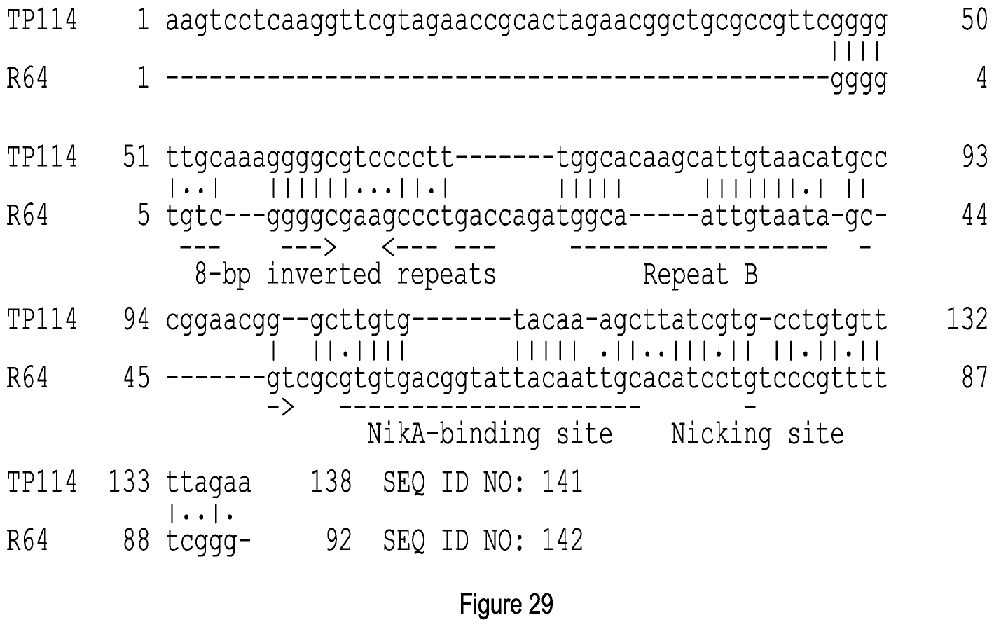

Identification of TP114's nicking site. In trans mobilization efficiency could be slightly perfected by immobilization of TP114, either by the deletion of the oriT or by integration in the donor's chromosome. Immobilization of the transfer machinery would prevent its spreading in target bacteria allowing for better biosafety by limiting the persistence of the transfer machinery in the environment. Also, an immobilized transfer machinery can still mobilize the genetic cargo in trans which allows the transfer of only the genes that need to be expressed in the recipients. However, oriT_{TP114} is located in an intergenic region between two diverging genes. This means that this region contains two unannotated promoters, one of which is responsible for the expression of the essential nickase gene nikB. Precise deletion is therefore required to avoid possible impairment of nikB expression of. Sequence comparison of oriT_{TP114} with other plasmids from the IncI2 family (Table 5) provided further information over sequence conservation. Among the tested plasmids, pChi7122-3 only showed homology for the first 138 bp of oriT_{TP114} sequence. It is therefore most likely that both the nikB promoter, and the nicking site, could be located in this portion of oriT_{TP114}. Bacterial promoters are usually composed of a −10 and −35 box and can optionally contain operator sequences. As nikB is located upstream of oriT_{TP114} and since a −10 box motif was found at position 13, the promoter of nikB is most likely located in the first 100 bp of oriT_{TP114}. In the IncI1 model plasmid R64, the core oriT_{R64} sequence was determined to be 92-bp long and the nicking site is located at position 77 at a highly conserved guanine. A Pairwise Sequence Alignment performed using EMBOSS Needle software (Rice et al., 2000) revealed 36% homology between the 138 first base pair of oriT_{TP114} and the minimal oriT_{R64} (FIG. 29). This alignment shows that important repeats, and NikA's binding site, were relatively well conserved between R64 and TP114. Using the position of these motifs in TP114, the nicking site was predicted to be G at position 124. To validate that the nicking site really was located at position 124, a 7-bp deletion from position 121 to 127 was performed, then cloned into a second vector, namely pNA02. Genetic cargo pNA02 is identical to pNA01 except for the 7-bp deletion and should therefore not be mobilized by TP114. In trans-mobilization of pNA02 was tested from KN01 containing TP114 to KN02 exactly as performed for pNA01. However, in trans mobilization was virtually abolished by the 7-bp deletion in pNA02's oriT_{TP114} (FIG. 28.B). This indicates that TP114's nicking site is located in this region.

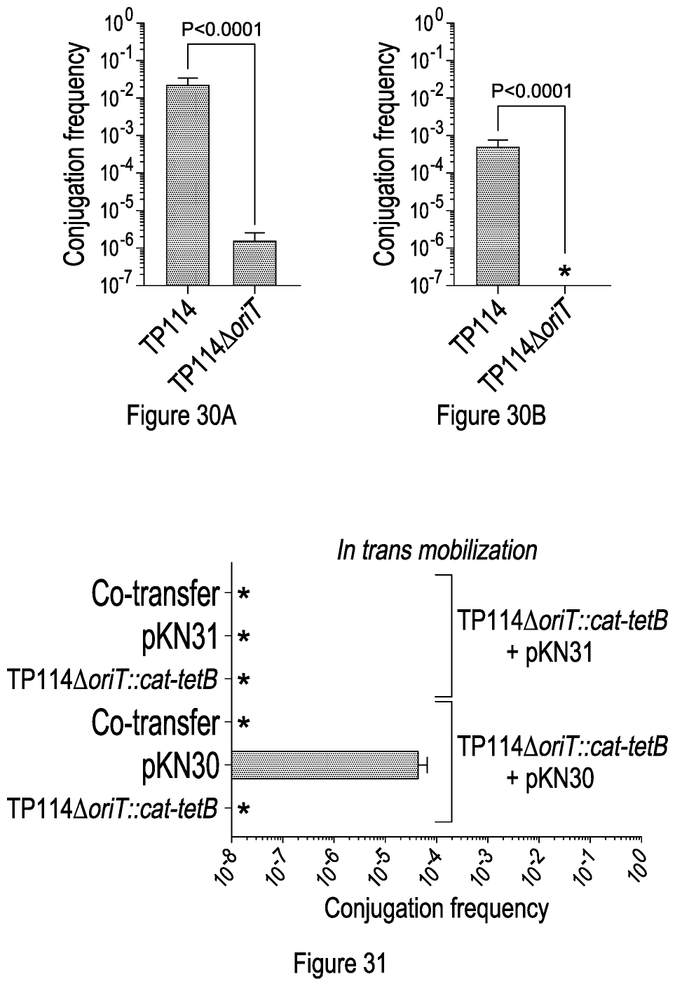

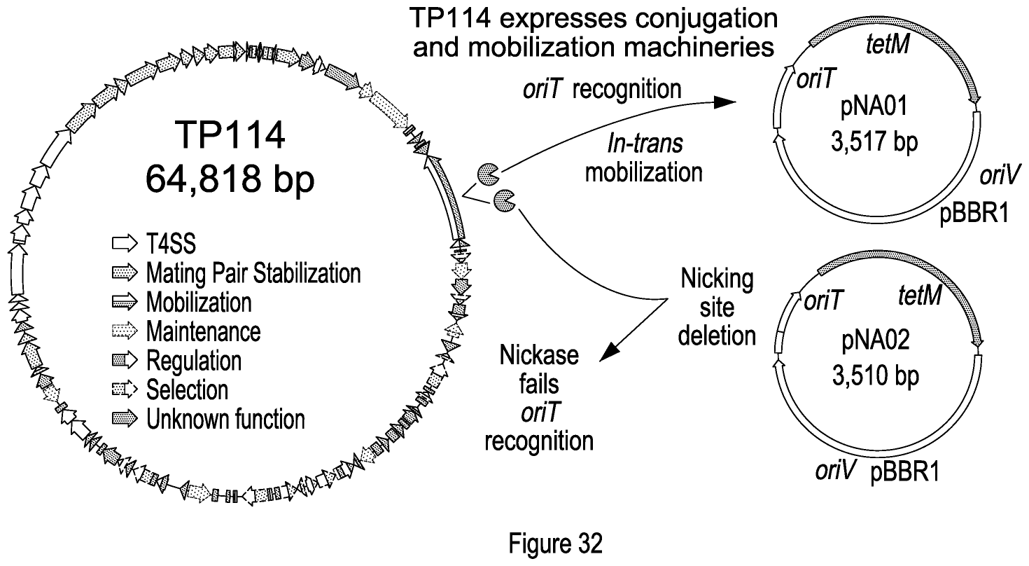

Deletion of oriT_{TP114} from the transfer machinery of TP114. As discussed above, the mutation of the oriT can be performed to immobilize a conjugative plasmid in a donor strain. In trans mobilization can benefit from this immobilization, otherwise, the transfer machinery and the genetic cargo will compete for transfer through the T4SS. The immobilization of TP114 was performed by recombineering-mediated insertion of a FRT flanked cat cassette creating a 138-bp deletion in oriT_{TP114} spanning position 116 to 254. This deletion covers the nicking site and should prevent the recognition of oriT_{TP114}, and hence the transfer machinery conjugation, while at the same time leaving the expression of nikB unaffected. TP114ΔoriT::cat-tetB was generated in MG1655Nx^{R} and transferred in KN01 by conjugation. Conjugation efficiency of TP114ΔoriT::cat-tetB was greatly impaired, but still yielded a few transconjugants (FIG. 30.A). Such transfer allowed exclusion mechanisms to isolate the TP114ΔoriT::cat-tetB from TP114 which could potentially still be present in the MG1655Nx^{R} strain. Therefore, a second test of conjugation efficiency was performed from KN01 towards MG1655Rf^{R}. This time, no transconjugants could be retrieved thereby showing the complete abolition of TP114's transfer capabilities (FIG. 30.B). Then, a kanamycin resistant derivative of pNA01 and pNA02, pKN30 and pKN31 respectively, were transformed into KN01+TP114ΔoriT::cat-tetB. Using the biocontained transfer machinery, pKN30, and pKN31 were mobilized in trans towards MG1655Nx^{R} (FIG. 31). Only pKN30 was able to transfer to MG1655Nx^{R}, thereby confirming that this confinement method is highly stringent. However, its conjugation efficiency was about 10-fold lower than TP114's. This could be further improved by relocating the relaxosome-associated genes from the transfer machinery to the genetic cargo vector. In summary, in trans mobilization, as demonstrated in the present example, required the relaxosome to be expressed from the transfer machinery (TP114). The relaxosome then recognized oriT_{TP114} on pNA01 and mediated its mobilization through the T4SS. However, the 7-bp deletion of the nicking site in pNA02 prevented the recognition and processing of oriT_{TP114} by the relaxosome thus impairing pNA02 transfer (FIG. 32).

### EXAMPLE IV—GENETIC CARGO DELIVERY AND APPLICATIONS

Strains, plasmids and growth conditions. All strains and plasmid used in this study are described in Table 1. All plasmid sequences are provided in the sequence appendix. Details about strains, plasmids, growth conditions, in vitro conjugation, feces and tissue processing, in vivo conjugation and statistical analysis are provided in the Material and Method section of the Example I.

Fluorescence measurements. To assess the efficacy of the gRNA-cas9 payloads at cutting a specific chromosomal DNA sequence, or a plasmidic DNA sequence, two types of bacterial targets were used. The first one, KN02, possesses a gene coding for a green fluorescent protein (Shaner et al., 2013) (NeonGreen™) integrated within its genome. The second one, KN03, carries a pT plasmid bearing a chloramphenicol resistance gene and a gfp gene (Ormö et al., 1996). Fluorescence was used as a proxy to measure the integrity of the bacterial genome of KN02 or of plasmid pT in KN03. Briefly, as long as the pT plasmid carrying the chloramphenicol resistance gene is devoid of any cut, it can be maintained in the bacterial host and GFP is expressed. As soon as the CRISPR-Cas9 system cuts the chloramphenicol resistance gene, pT is lost, thereby leading to loss of GFP fluorescence. To determine the efficiency of the COP system to cure pT, the number of green fluorescent and non-fluorescent colonies were counted on recipient and transconjugants selecting plates. Fluorescence was measured using a Typhoon FLA 9500 and images were analyzed using ImageFiji software.

Cell fluorescence induction. To limit possible negative effects of high levels fluorescent proteins on the fitness of the target bacteria, the genes coding for the fluorescent proteins were under the control of inducible promoters. In both genomic and plasmidic targets the fluorescent signal was inducible. The gfp gene from pT is under the repression of AraC, a protein which action is inhibited by arabinose (Guzman et al., 1995). gfp on pT is therefore inducible in the presence of 1% arabinose. In the case of the KN02 cells (chromosomal target), fluorescence is mediated by NeonGreen. E. coli KN02's NeonGreen gene is inducible with 1 mM IPTG (Lutz et al., 1997). NeonGreen emits green fluorescence with similar absorption and emission spectrum to GFP, but with brighter fluorescence (Shaner et al., 2013). Both plasmidic and genomic targets were confirmed for fluorescence emission under a transilluminator using blue light and in a cell sorter (FACSJazz) (data not shown).

Colony photography. To follow pT loss during both in vitro and in vivo experiments, colony fluorescence was detected using a Typhoon FLA 9500 on LB agar plates supplemented with 1% arabinose. To do so, two images were taken; one for the detection of GFP used a Low Pass Blue filter and a 473 nm laser, the other one for the BrightField image used a Low Pass Red filter and a 635 nm laser. Those two images were merged using ImageFiji software (Schindelin et al., 2012). Then, the green fluorescent and non-fluorescent colonies were manually counted.

Mortality rate evaluation by FACS. For in vitro experiments where the target was genomic, the mortality rate was investigated using a live/dead approach. Live and dead bacteria were discriminated using propidium iodide (PI) staining. Typically, PI is used to detect dead cells since it can only penetrate through compromised membranes (Davey, 2011). KN02's fluorescence was induced throughout the in vitro COP treatment. To detect dead cells, cells were stained with 30 μM PI in NaCl 0.85% for 15 minutes in the dark. Then, cells were washed twice and resuspended in NaCl 0.85% at a density allowing for rapid and accurate cell detection. Green and red fluorescence was immediately evaluated on 100 000 cells per sample using a FACSJazz cytometer.

4.1—In Vivo Use of the COP System to Transfer a Payload with Beneficial Effects on the Recipient Bacterium

The COP system can be used to deliver a genetic cargo beneficial to a target bacterium. Such a genetic cargo could encode genes that provide an advantageous phenotype to the recipient bacteria. Providing a phenotypic advantage to certain bacterial species could help rebalance a disturbed microbiota by helping under-represented species to proliferate. For example, this could be achieved by transferring genes allowing the use of a new carbon source, such as lactose. Providing lactose degradation enzymes would benefit the bacteria, but also the subject if lactose intolerant. Another example of beneficial genes that could be contained in the payload would be antibiotics resistance genes. Providing that certain bacteria needs to be enriched in a certain microbiota, transferring a resistance gene to these bacteria before antibiotics treatment would greatly enrich their population.

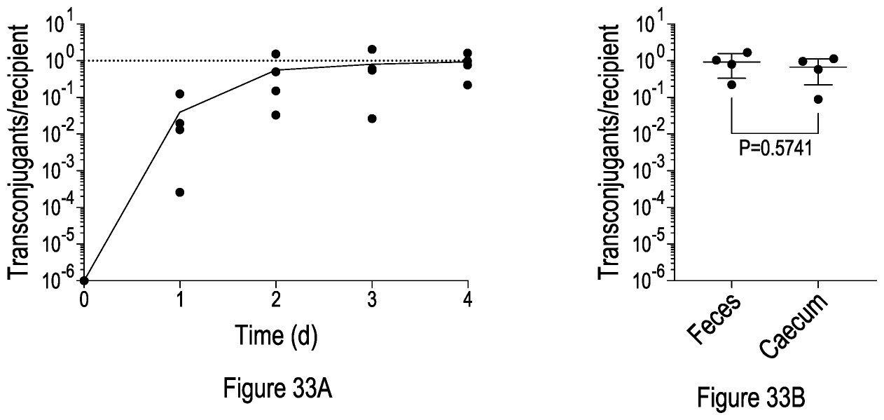

The COP system can transfer a payload providing beneficial phenotypic traits to target bacteria in vivo. The COP was tested for its ability to transfer a genetic cargo providing a beneficial phenotypic trait to target bacteria, and this, in the gut environment. To do so, the in vivo conjugation mouse model was used with KN01+TP114 as a donor to provide the kanamycin resistance gene to the KN02 recipient bacteria. The COP achieved high level of genetic cargo transfer within the first two days of the experiment as KN02 bacteria became resistant to kanamycin (FIG. 33.A). The results obtained were consistent between the caecum and feces (FIG. 33.B). Most of the recipient population gained the ability to resist kanamycin throughout the experiment, hereby showing that the COP could be used to transfer and deliver beneficial phenotypic traits to bacteria in vivo.

4.2—In Vivo Use of the COP to Transfer a Payload Detrimental for the Recipient Bacterium

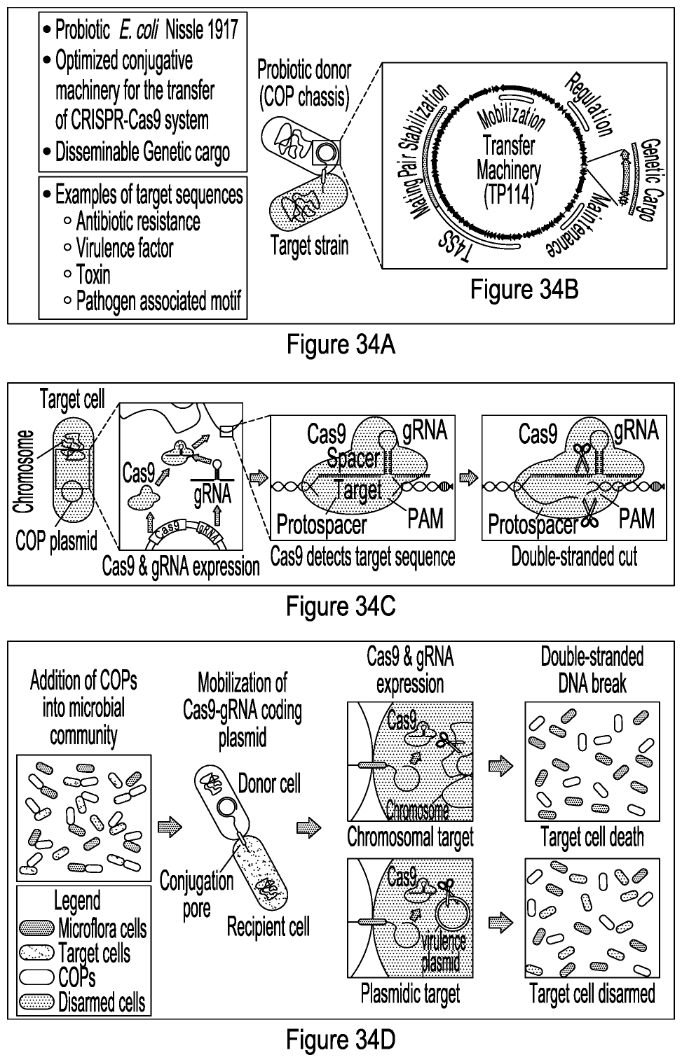

The COP system can be used to deliver the CRISPR-Cas9 system as a payload in vivo. In this example, the COP is used in the gut environment to deliver the CRISPR-cas9 system to inactivate specific genes into target bacteria. For this demonstration, the COP was composed of the probiotic EcN, (FIG. 34.A) with a conjugative delivery system having the transfer machinery and the genetic cargo located on the same vector (see cis mobilization in example III) (FIG. 34.B). In this configuration, because the transfer machinery and the genetic cargo are linked together, they are both transferred into the target bacterium which becomes a donor contributing to the exponential spreading of the cargo within the local microbiota. Once in a target recipient cell, the Cas9-gRNA payload is expressed and scans the genome for the specific target sequence determined by the tunable gRNA spacer's sequence. When a target sequence is detected, Cas9 catalyzes a double-stranded break into the cell's DNA (FIG. 34.C). The construction of the conjugative delivery system is detailed in example 3.1. Briefly, the genetic cargo and transfer machinery were linked together using the DROID method. The genetic cargo's payload was composed of the cas9 gene and one (Kill1) or three (Kill3) gRNAs. For this proof of concept, the gRNAs from the genetic cargos were designed to bind specifically to the chloramphenicol resistance gene (cat). The cat target sequence can be naturally found on a plasmid or on the chromosome of target bacteria. When cat is located on the chromosome of the target strain, the target cells were killed as a consequence of the double-strand breaks caused by CRISPR-cas9. On the other hand, when cat is located on a plasmid, the double-strand breaks induced by CRISPR-cas9 promoted the degradation of the plasmid and the cells were “disarmed” (FIG. 34.D).

4.2.1—In Vivo Use of the COP to Deliver a Payload that Sensitizes Bacteria to Antibiotics.

The COP can be used to deliver a payload that eliminates a phenotypic trait in target bacteria. One way the COPs can have a detrimental effect mediated by the transfer of its genetic cargo is by hindering the capacity of the target bacteria to express a given phenotype. One of the most infamous phenotype is the resistance to antibiotics. It could therefore be desirable to design a COP that induces the loss of antibiotic resistance genes in a bacterial population. The use of programmable endonuclease (e. g. cas9) holds great promise for the targeting and elimination of antibiotic resistance genes responsible for the emergence of extremely resistant pathogenic bacteria. Antibiotic resistance genes can be found on both bacterial chromosomes and plasmids. When those genes are located on a horizontally transferable genetic element, like conjugative and mobilizable plasmids, they are more problematic. In this particular scenario, the resistance phenotype is transferable to other species of bacteria. The use of the COP bearing CRISPR-Cas9 allows the targeting of these resistance genes and the disarmament of bacteria bearing such mobile genetic elements in vivo.

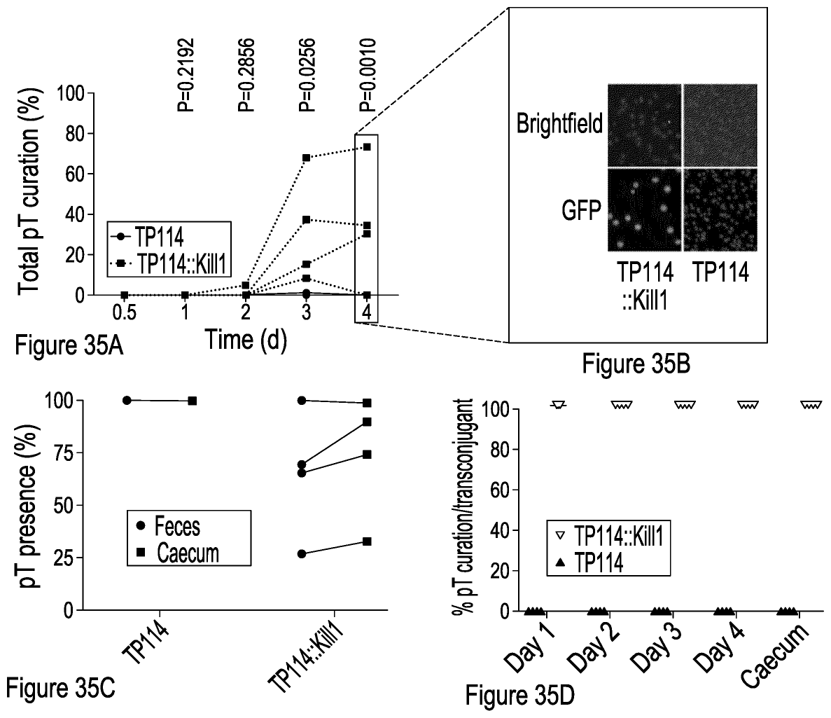

The COP can deliver a payload that cures antibiotic resistance plasmids in vivo. The COP was used during an in vivo conjugation experiment to test whether it could deliver a payload in order to eliminate antibiotic resistance plasmids from target bacteria. To do so, the COP was tested for its ability to deliver a payload capable of eliminating pT from a commensal E. coli strain in the murine intestinal tract. KN03 bearing pT was introduced in mice 12 hours prior to KN01 bearing TP114 or KN01 bearing TP114::Kill1. The presence of pT in the recipient cells was monitored by fluorescence on selective LB plates. A single administration of the COP system could clear as much as 73% of the target plasmid throughout the four days of the experiment (FIGS. 35.A and B). No plasmid loss was observed in the control group (KN01+TP114) and all transconjugants of the COP system were devoid of the target plasmid (FIG. 35.C). Results from feces samples were consistent with frequencies found in the caecum of the mice at the end of the experiment (FIG. 35.D). These results indicated that it was possible to specifically cure a resistance plasmid from a cell in vivo in the gut microbiota using the COP system to deliver a payload containing the CRISPR-cas9, hereby causing the loss of antibiotic resistance phenotype.

42.2—In Vivo Use of the COP System to Deliver a Payload in Order to Selectively Kill Bacteria.

The COP system can be used as an alternative to conventional antibiotic drugs. The use of CRISPR-Cas9 to target and eliminate specific bacteria in a mixed community is an application of great interest since the emergence of antibiotic resistance threatens our ability to treat bacterial infections. Using TP114::Kill1, the COP can target chromosomal sequences into pathogenic bacteria's genomes to eliminate them. Since most bacteria are unable to perform Non-Homologous End Joining (NHEJ), a Cas9 mediated blunt double-stranded cut in their chromosome can result in death. In this in vivo example, a specific strain of bacteria is successfully targeted and eliminated from the community without affecting other closely related strains. To test the ability of the COP to deliver a payload to eliminate specific bacteria from a complex bacterial community, a set of closely related target and non-target strains was required. For these experiments, KN01ΔdapA was used as the donor strain, KN02, which carries a targeted chromosomal cat gene was used as the target strain, whereas KN03, which carries a chromosomal tetB gene instead of cat was used as the non-target strain.

4.2.2.1—Prophylactic Use of the COP System to Selectively Kill Bacteria In Vivo.

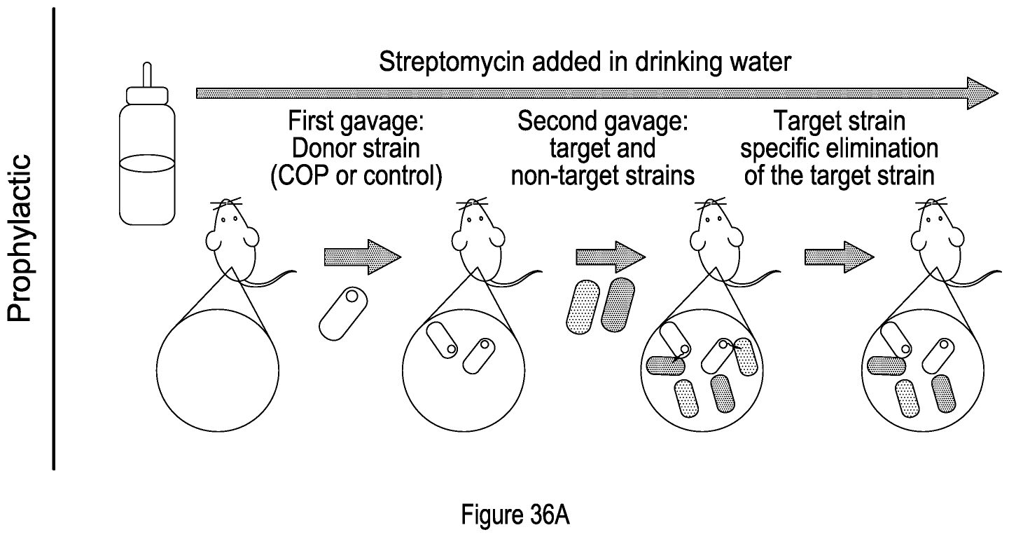

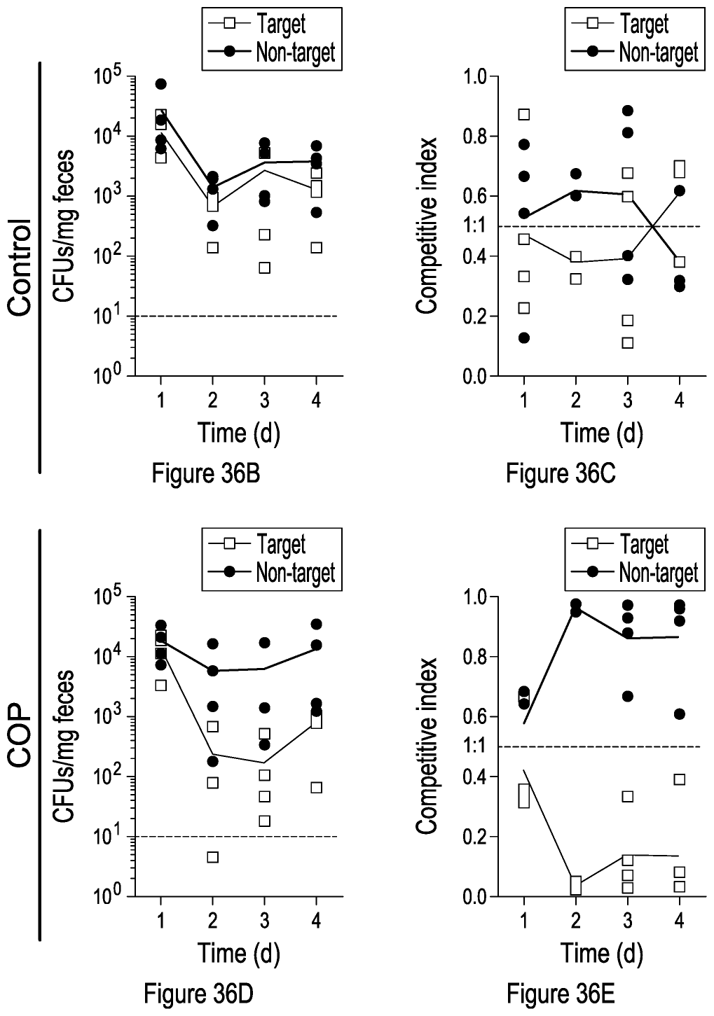

Applications of prophylactic use for the COP TP114::Kill1 system. The prophylactic treatment implies a daily uptake of the COP to avoid infections or the spreading of unwanted bacterial strain. This type of treatment could be useful in situations where a subject is susceptible to be exposed to a certain type of bacterial infection, e.g. when traveling, or while recovering from an antibiotic treatment. Prophylactic use of COPs could also improve health by targeting antibiotic resistant bacteria thereby lowering the chances of antibiotic resistant infections. A mouse model for the investigation of such prophylactic treatment was designed. Mice were first fed with the COP system bearing TP114::Kill1 or TP114 (as a control), and then were subjected 12 hours later to a 1:1 mix of target and non-target bacteria (respectively KN02 and KN03). The respective abundance of the target and the non-target bacteria was then followed over four days in feces (FIG. 36.A).

The COP system can be administered prophylactically to deliver a payload that specifically eliminates an invading target bacterium in vivo. The murine prophylactic treatment was followed strictly using COP KN01+TP114::Kill1 or KN01+TP114 (control placebo treatment). The COP prophylactic treatment resulted in as much as 13-fold specific decrease of target strain's abundance as compared with the non-target strain, and this, in a single day. A clear drop in raw CFUs count of the target strain was observed with only a single dose of COP KN01 bearing TP114::Kill1 prophylactic treatment (compare FIGS. 36.B and 36.D). A competitive ratio was calculated between the target strain and the non-target strain by comparing the CFU count between the two strains. For example, the target strain competitive ratio was calculated by dividing the CFU count of the target strain by that of the total recipient strains CFU. The equivalent was done to calculate the non-target strain competitive ratio. Competitive ratio compares the fitness of the two strains to highlight the impact of the COP system. The strain competitive ratios were consistent with raw CFUs with a clear difference between the recipient strains at days 2, 3 and 4 for the COP KN01 bearing TP114::Kill1 treatment, but not the control (compare FIGS. 36.C and 36.E). The difference in strain colonization suggests that the COP technology can efficiently and specifically eliminate the target strain in vivo, allowing the non-target strain to thrive. This showed that the COP system could be used prophylactically to deliver a payload in vivo in order to specifically prevent pathogenic bacteria to invade the gut microbiome.

4.2.2.2—Therapeutic Use of the COP System to Selectively Kill Bacteria In Vivo.

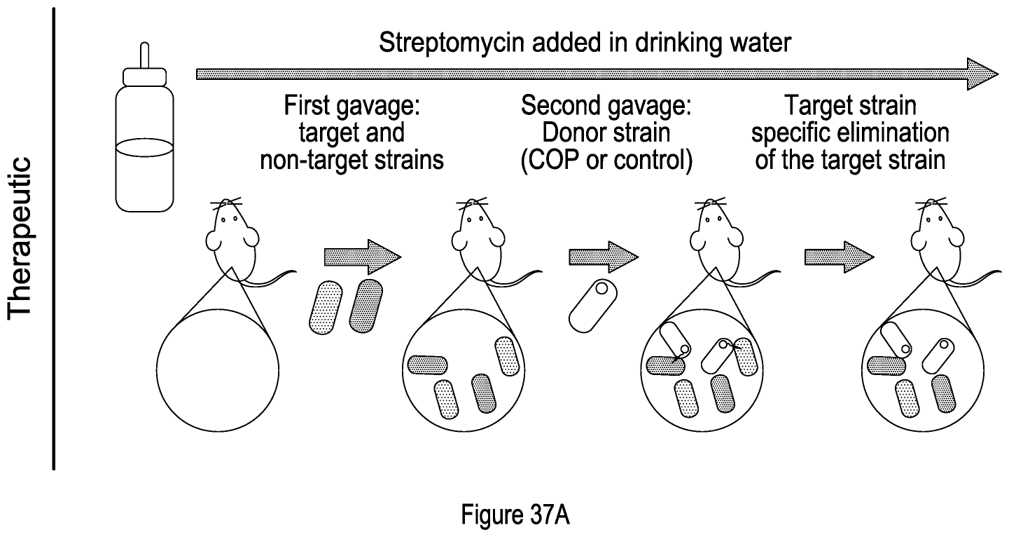

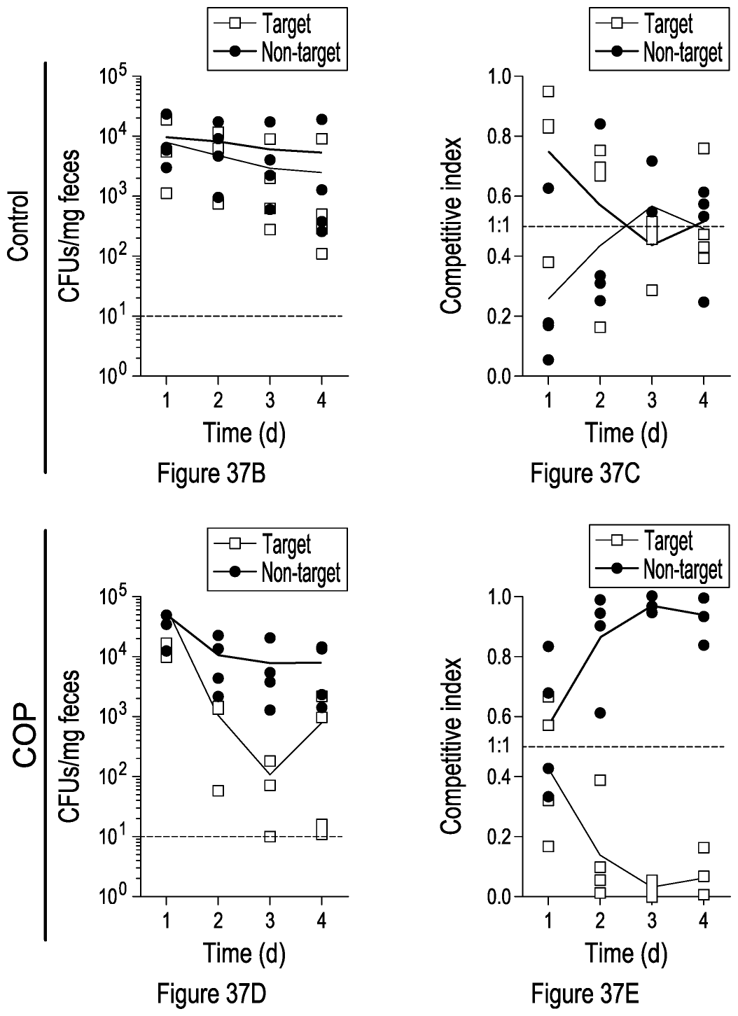

Examples of therapeutic uses for the COP in vivo. The therapeutic use of the COP implies that the administration of COP occurs after the detection of a given pathogenic bacteria. The COP is administered to eliminate or inactivate the pathogenic bacteria. This type of treatment could be useful in situations where it is preferable to induce mortality in specific pathogen cells. In fact, the high specificity of the COP system would prevent the establishment of opportunist pathogens and would limit the apparition of pathologies such as antibiotic-associated diarrhea. Furthermore, treatment of a subject could be preferable in situations where the target bacteria are resistant to all known antibiotics. Nonetheless, high specificity of the COP could allow precise engineering of the gut microbiota in dysbiosis-affected subjects by efficiently targeting over-represented species in an individual. A mouse model for the investigation of the efficacy of such therapeutic treatment was devised. The mice were first fed with a 1:1 mix of target and non-target bacteria (respectively KN02 and KN03) and 12 hours later, were treated with the COP containing either the TP114::Kill1 system or TP114 (control). The abundance of target and non-target bacteria was followed in feces over four days (FIG. 37.A).

The COPs can deliver a payload to eliminate a target bacterium in vivo. The murine therapeutic treatment was followed strictly using COP KN01 bearing TP114::Kill1 or KN01 bearing TP114 (control placebo treatment). A single dose of COP therapeutic treatment yielded outstanding results with as much as 2,116-fold diminution of target strain as compared with the non-target strain within just two days. Significant diminution in raw CFUs was observed on days 2, 3 and 4 with COP KN01 bearing TP114::Kill1-treated mice but not with placebo-treated mice (compare FIGS. 37.B and 37.D). Target and non-target competitive indexes (as calculated in the prophylactic treatment section) showed clear dominance of the non-target strain for COP KN01 bearing TP114::Kill1 treated mice, but not for the control group (compare FIGS. 37.C and 37.E). This showed that the COP technology could be used therapeutically to deliver a payload specifically designed to eliminate a bacterium in vivo.

4.3—In Vivo Use of the COP System for the Transfer of a Payload with Mixed Effects on Bacterial Populations

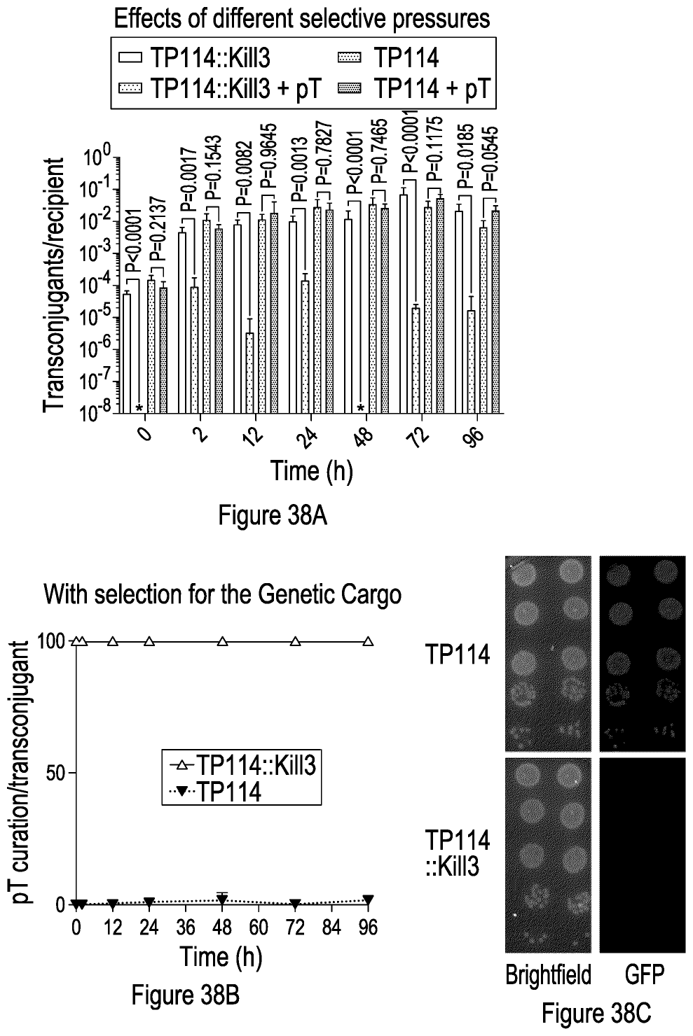

Utilization of a payload with beneficial and detrimental effects on the recipient bacteria. Using TP114::Kill3 delivery system, it is possible to exploit the transfer of both beneficial gene (kanamycin resistance) and detrimental gene (Cas9-gRNA) in a same genetic cargo to manipulate a population of bacteria. For instance, by transferring the kanamycin resistance gene of TP114::Kill3 into target bacteria and exposing those cells to kanamycin, a selective pressure towards the acquisition of the genetic cargo occurs, hereby forcing the recipient bacteria to be subjected to the effects of CRISPR-Cas9. The plasmid pT that contains the target gene cat and expresses GFP was used to demonstrate this approach (FIG. 21.E). Since TP114 transfers at frequencies around 1% on solid medium in vitro, CRISPR-Cas9 can potentially mediate plasmid loss in about 1% of the population without antibiotic selection. TP114::Kill3 transfer was measured towards KN03 containing pT by selecting either TP114:Kill3 only or both TP114::Kill3 and pT. As a control, TP114 was transferred to KN03 bearing pT to verify that any plasmid curation was not resulting from plasmid incompatibility (FIG. 38.A). The transfer frequency was found to be significantly lower when selecting for both plasmids only with TP114::Kill3 but not with TP114, thereby suggesting that plasmid curation is due to the gRNA-Cas9 system present on TP114::Kill3. Using GFP signals, it was possible to determine the number of recipient bacteria cured of pT following the COP treatment with TP114::Kill3. This number varied between 1 and 7% in the absence of selection. However, in the presence of kanamycin to select for TP114::Kill3 in the recipient cells, plasmid curation reached 100% (FIGS. 38.B and 38.C). pT loss was also confirmed by subculturing 63 transconjugant colonies on selective antibiotics media (21 for TP114 and 42 for TP114::Kill3). Only transconjugants from TP114::Kill3 were successfully confirmed for pT loss (42/42). Conjugation of TP114 WT plasmid into a KN03 bearing pT strain did not lead to pT loss in transconjugants, showing that plasmid loss was due to the expression of the genetic cargo and not plasmid incompatibility. Using this strategy, the efficiency of the system could be enhanced from ˜1% to 100% in vitro. This type of strategy could be scaled up to use several gRNA that targets several resistance genes at a time. This would allow the system to eliminate several antibiotics resistance genes while spreading only one resistance.

It is therefore expected that in some embodiments, it could be desirable to spread a resistance gene and to couple the COP with an antibiotic treatment.

4.4—COP can Deliver a Payload Across Species

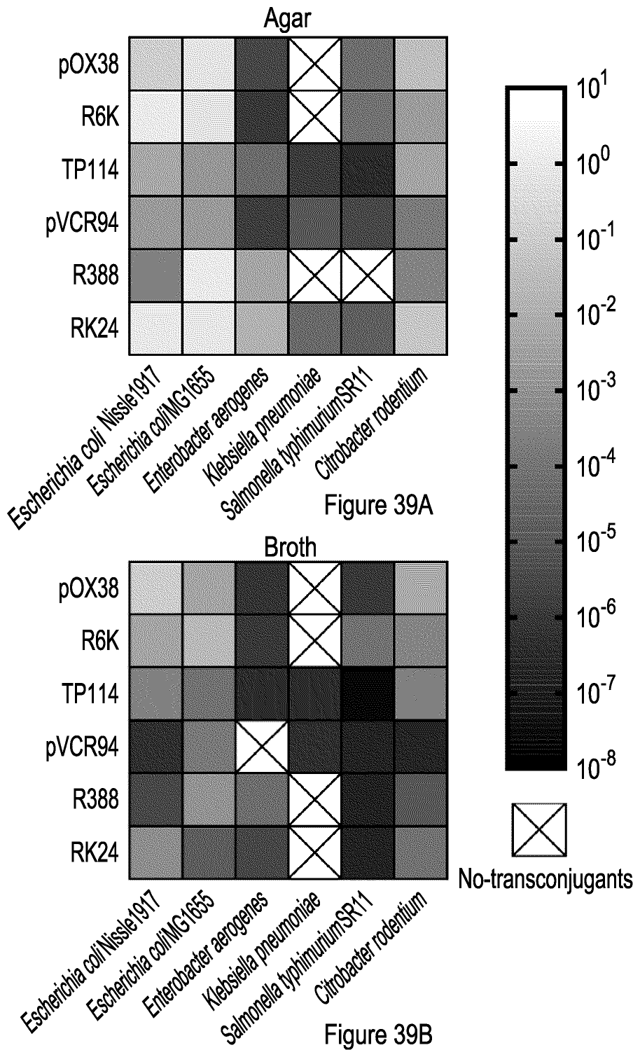

COP can deliver a phenotypic trait as a payload across species. The ability of the COP to transfer a beneficial trait to other species of bacteria was first tested by conjugation between E. coli KN01ΔdapA bearing TP114 as the COP and diverse bacterial species as individual recipients. The recipient bacteria (Salmonella enterica substr. Typhimurium SR-11, E. coli MG1655Nx^{R}, Citrobacter rodentium DBS100, Enterobacter aerogenes ATCC 35029, Klebsiella pneumoniae ATCC 13883, E. coli KN02) were chosen as part of the Enterobacteriaceae family, a family of bacteria most frequently found in the gut microbiome. The process was repeated with alternative transfer machinery (conjugative plasmids pVCR94, RK24, pOX38, R6K, R388) under solid and liquid mating conditions (FIGS. 39.A and 39.B). Most of the transfer machineries were able to transfer the antibiotic resistance across species, highlighting the broad host range of these genetic constructs. The transfer frequency of the payload varied between recipients, but all recipient bacteria could successfully acquire the phenotypic trait.

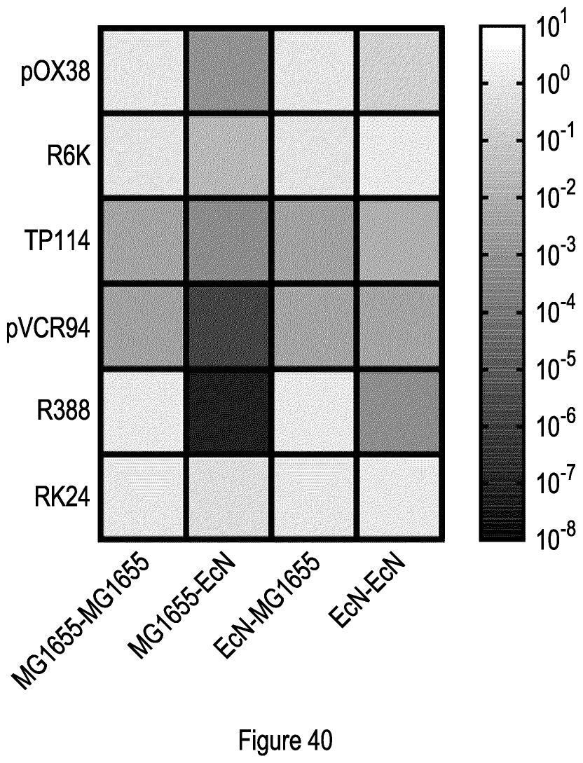

Compatibility in Restriction-Modification (RM) systems benefits Horizontal Gene Transfer (HGT). The discrepancy between transfer frequencies was next investigated. One of the major barriers to HGT is the compatibility of restriction-modification systems. As such, the stability of the transferred genetic cargo depends on the ability of the recipient bacteria to target and eliminate the genetic construct. The targeting of a new DNA molecule often relies on RM system, in which DNA molecules are specifically modified to avoid recognition by specific nucleases. As such, compatibility between the probiotic donor's modification system and the recipient's restriction system can influence the recognition rate of the genetic cargo by specific nucleases. It was noted that the transfer of DNA by conjugation seemed limited from E. coli MG1655Nx^{R} to EcN and from EcN to MG1655Nx^{R}, but not between two MG1655 or EcN strains for most tested conjugative systems (FIG. 40). This result suggested that MG1655's modification system was not compatible with EcN's restriction system and vice versa. Therefore, choosing a donor bacterium that has compatible modification systems with the recipient bacteria or engineering one can enhance the payload's stability in the recipient bacteria. Alternatively, a gene encoding an anti-restriction protein can be added to the genetic cargo to enhance its transmissibility between incompatible bacteria. One of such anti-restriction gene is found on RK24 (kclA), thereby explaining the absence of drop for conjugation from MG1655 to EcN. The anti-restriction system of RK24 allowed generally better transfer frequency of its antibiotic resistance gene across several species (FIG. 39.A). Therefore, adding an anti-restriction protein to the COP system could be desirable to improve the delivery of cargos across species.

Conjugative plasmids are isolated genetic constructs that functions mostly independently from the donor strain. Since conjugative plasmids can spread across several genera of bacteria, their independence from the host chromosome to achieve DNA transfer is important for their persistence through time. While the cell provides resources to a conjugative plasmid, such a plasmid is often auto-regulated and expresses most of the proteins necessary for its adequate functioning. As such, conjugative plasmid's efficiency to transfer should not be affected significantly by the host bacterium. To test this hypothesis, two distantly related E. coli strains MG1655Nx^{R} and E. coli KN01 were chosen to compare conjugation efficiency within the strains. The results from FIG. 40 suggested that the conjugation efficiency was not dependent on the donor strain but is rather the same in both tested strains. The transfer efficiency of all tested conjugative plasmids was the same in both E. coli donor strain, even though those two strains are distantly related. Therefore, the COP system could be easily adapted to operate in a plethora of probiotic hosts.

### REFERENCES

Alikhan N F, Petty N K, Ben Zakour N L, Beatson S A. BLAST Ring Image Generator (BRIG): simple prokaryote genome comparisons. BMC Genomics. 2011 Aug. 8; 12:402. doi: 10.1186/1471-2164-12-402. PubMed PMID: 21824423.
Allard N, Garneau D, Poulin-Laprade D, Burrus V, Brzezinski R, Roy S. A diaminopimelic acid auxotrophic Escherichia coli donor provides improved counterselection following intergeneric conjugation with actinomycetes. Can J Microbiol. 2015 August; 61(8):565-74. doi: 10.1139/cjm-2015-0041. Epub 2015 Jun. 3. PubMed PMID: 26166710.
Altschul S F, Gish W, Miller W, Myers E W, Lipman D J. Basic local alignment search tool. J Mol Biol. 1990 Oct. 5; 215(3):403-10. PubMed PMID: 2231712.
Andrew S. (2010). FastQC: a quality control tool for high throughput sequence data. Available online at: http://www.bioinformatics.babraham.ac.uk/projects/fastqc/
Aziz R K, Bartels D, Best A A, DeJongh M, Disz T, Edwards R A, Formsma K, Gerdes S, Glass E M, Kubal M, Meyer F, Olsen G J, Olson R, Osterman A L, Overbeek R A, McNeil L K, Paarmann D, Paczian T, Parrello B, Pusch G D, Reich C, Stevens R, Vassieva O, Vonstein V, Wilke A, Zagnitko O. The RAST Server: rapid annotation using subsystems technology. BMC Genomics. 2008 Feb. 8; 9:75. doi: 10.1186/1471-2164-9-75. PubMed PMID: 18261238.
Born T L, Blanchard J S. Structure/function studies on enzymes in the diaminopimelate pathway of bacterial cell wall biosynthesis. Curr Opin Chem Biol. 1999 October; 3(5):607-13. Review. PubMed PMID: 10508663.
Benjamin W H Jr, Turnbough C L Jr, Posey B S, Briles D E. Salmonella typhimurium virulence genes necessary to exploit the Itys/s genotype of the mouse. Infect Immun. 1986 March; 51(3):872-8. PubMed PMID: 3512437.
Bolger A M, Lohse M, Usadel B. Trimmomatic: a flexible trimmer for Illumina sequence data. Bioinformatics. 2014 Aug. 1; 30(15):2114-20. doi: 10.1093/bioinformatics/btu170. Epub 2014 Apr. 1. PubMed PMID: 24695404.
Bradley D E. Characteristics and function of thick and thin conjugative pili determined by transfer-derepressed plasmids of incompatibility groups 11, 12, 15, B, K and Z. J Gen Microbiol. 1984 June; 130(6):1489-502.
Bradley D E, Taylor D E, Cohen D R. Specification of surface mating systems among conjugative drug resistance plasmids in Escherichia coli K-12. J Bacteriol. 1980 September; 143(3):1466-70. PubMed PMID: 6106013.
Carraro N, Matteau D, Luo P, Rodrigue S, Burrus V. The master activator of IncA/C conjugative plasmids stimulates genomic islands and multidrug resistance dissemination. PLoS Genet. 2014 Oct. 23; 10(10):e1004714. doi: 10.1371/journal.pgen.1004714. eCollection 2014 October PubMed PMID: 25340549.
Ceccarelli D, Daccord A, René M, Burrus V. Identification of the origin of transfer (oriT) and a new gene required for mobilization of the SXT/R391 family of integrating conjugative elements. J Bacteriol. 2008 August; 190(15):5328-38. doi: 10.1128/JB.00150-08. Epub 2008 Jun. 6. PubMed PMID: 18539733.
Datsenko K A, Wanner B L. One-step inactivation of chromosomal genes in Escherichia coli K-12 using PCR products. Proc Natl Acad Sci USA. 2000 Jun. 6; 97(12):6640-5. PubMed PMID: 10829079.
Datta S, Costantino N, Court D L. A set of recombineering plasmids for gram-negative bacteria. Gene. 2006 Sep. 1; 379:109-15. Epub 2006 May 4. PubMed PMID: 16750601.
Davey H M. Life, death, and in-between: meanings and methods in microbiology. Appl Environ Microbiol. 2011 Aug. 15; 77(16):5571-6. doi: 10.1128/AEM.00744-11. Epub 2011 Jun. 24. Review. PubMed PMID: 21705550.
Ferrières L, Hémery G, Nham T, Guérout A M, Mazel D, Beloin C, Ghigo J M. Silent mischief: bacteriophage Mu insertions contaminate products of Escherichia coli random mutagenesis performed using suicidal transposon delivery plasmids mobilized by broad-host-range RP4 conjugative machinery. J Bacteriol. 2010 December; 192(24):6418-27. doi: 10.1128/JB.00621-10. Epub 2010 Oct. 8. PubMed PMID: 20935093.
Furuya N, Komano T. Initiation and termination of DNA transfer during conjugation of Incl1 plasmid R64: roles of two sets of inverted repeat sequences within oriT in termination of R64 transfer. J Bacteriol. 2000 June; 182(11):3191-6. PubMed PMID: 10809699.
Goryshin I Y, Miller J A, Kil Y V, Lanzov V A, Reznikoff W S. Tn5/IS50 target recognition. Proc Natl Acad Sci USA. 1998 Sep. 1; 95(18):10716-21. PubMed PMID: 9724770.
Green R, Rogers E J. Transformation of chemically competent E. coli. Methods Enzymol. 2013; 529:329-36. doi: 10.1016/6978-0-12-418687-3.00028-8. PubMed PMID:24011059.
Guzman L M, Belin D, Carson M J, Beckwith J. Tight regulation, modulation, and high-level expression by vectors containing the arabinose PBAD promoter. J Bacteriol. 1995 July; 177(14):4121-30. PubMed PMID: 7608087.
Haeussler M, Raney B J, Hinrichs A S, Clawson H, Zweig A S, Karolchik D, Casper J, Speir M L, Haussler D, Kent W J. Navigating protected genomics data with UCSC Genome Browser in a Box. Bioinformatics. 2015 Mar. 1; 31(5):764-6. doi: 10.1093/bioinformatics/btu712. Epub 2014 Oct. 27. PubMed PMID: 25348212.
Ishiwa A, Komano T. Thin pilus PilV adhesins of plasmid R64 recognize specific structures of the lipopolysaccharide molecules of recipient cells. J Bacteriol. 2003 September; 185(17):5192-9. PubMed PMID: 12923092.
Kotula J W, Kerns S J, Shaket L A, Siraj L, Collins J J, Way J C, Silver P A. Programmable bacteria detect and record an environmental signal in the mammalian gut. Proc Natl Acad Sci USA. 2014 Apr. 1; 111(13):4838-43. doi: 10.1073/pnas.1321321111. Epub 2014 Mar. 17. PubMed PMID: 24639514.
Kvitko B H, Bruckbauer S, Prucha J, McMillan I, Breland E J, Lehman S, Mladinich K, Choi K H, Karkhoff-Schweizer R, Schweizer H P. A simple method for construction of pir+ Enterobacterial hosts for maintenance of R6K replicon plasmids. BMC Res Notes. 2012 Mar. 20; 5:157. doi: 10.1186/1756-0500-5-157. PubMed PMID: 22433797.
Li, H (2013) Aligning sequence reads, clone sequences and assembly contigs with BWA-MEM. arXiv:1303.3997v1 [q-bio.GN].
Lutz R, Bujard H. Independent and tight regulation of transcriptional units in Escherichia coli via the LacR/O, the TetR/O and AraC/11-12 regulatory elements. Nucleic Acids Res. 1997 Mar. 15; 25(6):1203-10. PubMed PMID: 9092630.
Marchler-Bauer A, Bo Y, Han L, He J, Lanczycki C J, Lu S, Chitsaz F, Derbyshire M K, Geer R C, Gonzales N R, Gwadz M, Hurwitz D I, Lu F, Marchler G H, Song J S, Thanki N, Wang Z, Yamashita R A, Zhang D, Zheng C, Geer L Y, Bryant S H. CDD/SPARCLE: functional classification of proteins via subfamily domain architectures. Nucleic Acids Res. 2017 Jan. 4; 45(D1):D200-D203. doi: 10.1093/nar/gkw1129. Epub 2016 Nov. 29. PubMed PMID: 27899674.
McKenzie G J, Craig N L. Fast, easy and efficient: site-specific insertion of transgenes into enterobacterial chromosomes using Tn7 without need for selection of the insertion event. BMC Microbiol. 2006 Apr. 28; 6:39. PubMed PMID: 16646962.
Metcalf W W, Jiang W, Wanner B L. Use of the rep technique for allele replacement to construct new Escherichia coli hosts for maintenance of R6K gamma origin plasmids at different copy numbers. Gene. 1994 Jan. 28; 138(1-2):1-7. PubMed PMID: 8125283.
Ormo M, Cubitt A B, Kallio K, Gross L A, Tsien R Y, Remington S J. Crystal structure of the Aequorea victoria green fluorescent protein. Science. 1996 Sep. 6; 273(5280):1392-5. PubMed PMID: 8703075.
Papagiannitsis C C, Tzouvelekis L S, Kotsakis S D, Tzelepi E, Miriagou V. Sequence of pR3521, an IncB plasmid from Escherichia coli encoding ACC-4, SCO-1, and TEM-1 beta-lactamases. Antimicrob Agents Chemother. 2011 January; 55(1):376-81. doi: 10.1128/AAC.00875-10. Epub 2010 Oct. 18. PubMed PMID: 20956594.
Praszkier J, Pittard A J. Control of replication in I-complex plasmids. Plasmid. 2005 March; 53(2):97-112. Review. PubMed PMID: 15737397.
Rice P, Longden I, Bleasby A. EMBOSS: the European Molecular Biology Open Software Suite. Trends Genet. 2000 June; 16(6):276-7. PubMed PMID: 10827456.
Rodrigue S, Materna A C, Timberlake S C, Blackburn M C, Malmstrom R R, Alm E J, Chisholm S W. Unlocking short read sequencing for metagenomics. PLoS One. 2010 Jul. 28; 5(7):e11840. doi: 10.1371/journal.pone.0011840. PubMed PMID: 20676378.
Ronchel M C, Ramos J L. Dual system to reinforce biological containment of recombinant bacteria designed for rhizoremediation. Appl Environ Microbiol. 2001 June; 67(6):2649-56. PubMed PMID: 11375176; PubMed Central PMCID: PMC92920.
Schindelin J, Arganda-Carreras I, Frise E, Kaynig V, Longair M, Pietzsch T, Preibisch S, Rueden C, Saalfeld S, Schmid B, Tinevez J Y, White D J, Hartenstein V, Eliceiri K, Tomancak P, Cardona A. Fiji: an open-source platform for biological-image analysis. Nat Methods. 2012 Jun. 28; 9(7):676-82. doi: 10.1038/nmeth.2019. PubMed PMID: 22743772.
Seiffert S N, Carattoli A, Schwendener S, Collaud A, Endimiani A, Perreten V. Plasmids Carrying bla(CMY-2/4) in Escherichia coli from Poultry, Poultry Meat, and Humans Belong to a Novel IncK Subgroup Designated IncK2. Front Microbiol. 2017 Mar. 15; 8:407. doi: 10.3389/fmicb.2017.00407. eCollection 2017. PubMed PMID: 28360894.
Sekizuka T, Kawanishi M, Ohnishi M, Shima A, Kato K, Yamashita A, Matsui M, Suzuki S, Kuroda M. Elucidation of quantitative structural diversity of remarkable rearrangement regions, shufflons, in IncI2 plasmids. Sci Rep. 2017 Apr. 19; 7(1):928. doi: 10.1038/s41598-017-01082-y. PubMed PMID: 28424528.
Shaner N C, Lambert G G, Chammas A, Ni Y, Cranfill P J, Baird M A, Sell B R, Allen J R, Day R N, Israelsson M, Davidson M W, Wang J. A bright monomeric green fluorescent protein derived from Branchiostoma lanceolatum. Nat Methods. 2013 May; 10(5):407-9. doi: 10.1038/nmeth.2413. Epub 2013 Mar. 24. PubMed PMID: 23524392.
Sonnenborn, U. & Schulze, J. The non-pathogenic Escherichia coli strain Nissle 1917—features of a versatile probiotic. Microb. Ecol. Health Dis. 21, 122-158 (2009).
Venturini C, Hassan K A, Roy Chowdhury P, Paulsen I T, Walker M J, Djordjevic S P. Sequences of two related multiple antibiotic resistance virulence plasmids sharing a unique IS26-related molecular signature isolated from different Escherichia coli pathotypes from different hosts. PLoS One. 2013 Nov. 4; 8(11):e78862. doi: 10.1371/journal.pone.0078862. eCollection 2013. PubMed PMID: 24223859.

1.-49. (canceled)

50. A method for transferring, in vivo in a subject suspected of having a recipient bacterium, a genetic cargo from a conjugative bacterial host cell to the recipient bacterium, the method comprising administering an effective amount of a conjugative recombinant bacterial host cell to the subject under conditions to allow the transfer of the genetic cargo to the recipient bacterium, wherein the conjugative host cell comprises:
the genetic cargo, wherein the genetic cargo comprises a transport module operatively associated with a payload module;
a type IV secretion system module;
a mating pair stabilization module comprising a type IV adhesion pilus, the type IV adhesion pilus comprising an adhesin; and
a mobilization module encoding a transport machinery,
wherein the transport module is capable of being recognized by transport machinery encoded by the mobilization module.

51. The method of claim 50, wherein the conjugative bacterial host cell is a probiotic bacterial host cell.

52. The method of claim 50, wherein the conjugative bacterial host cell is an enteric bacterium.

53. The method of claim 50, wherein the modification system of the conjugative bacterial host cell is substantially similar to the restriction-modification system of the recipient bacterium.

54. The method of claim 50, wherein the payload module encodes a heterologous protein.

55. The method of claim 54, wherein the heterologous protein is a therapeutic protein, allows for the production or the degradation of a metabolite or is a nuclease.

56.-57. (canceled)

58. The method of claim 55, wherein the nuclease is a clustered regularly interspaced short palindromic repeat (CRISPR) associated DNA-binding (Cas) protein and the payload module further encodes a guide RNA (gRNA) molecule recognizable by the Cas protein.

59. The method of claim 50, wherein the genetic cargo is a guide RNA (gRNA) molecule, wherein the gRNA is substantively complementary to a DNA or a RNA molecule in the recipient bacterium.

60. The method of claim 59, wherein the DNA molecule is a gene in the recipient bacterium.

61. The method of claim 60, wherein the gene encodes a virulence factor in the recipient bacterium.

62. The method of claim 61, wherein the gene encodes, in the recipient bacterium, a protein involved in a resistance to an antibiotic, a toxin or a pilus in the recipient bacterium.

63. The method of claim 50 for the treatment or the alleviation of symptoms of a dysbiosis or an infection caused by the recipient bacterium.

64. The method of claim 50, wherein the conjugative bacterial host cell comprises a transfer machinery located on a second extrachromosomal vector, wherein:
the transfer machinery comprises the type IV secretion system module, the mating pair stabilization module and a second vegetative replication module;
the conjugative bacterial host cell comprises a second maintenance module encoding a second replication machinery; and
the second vegetative replication module is capable of being recognized by the second replication machinery encoded by the second maintenance module.

65. The method of claim 50, wherein the mating pair stabilization module further comprises a shufflase for modifying a shufflon associated with the gene encoding the adhesin.

66. The method of claim 50, wherein the subject is a human subject or an animal subject.

67. The method of claim 50, comprising a transfer machinery located in the bacterial chromosome, wherein the transfer machinery comprises the type IV secretion system module, the mating pair stabilization module and the mobilization module.
